(19) United States
(12) Patent Application Publication
(10) Pub. No.: US 2026/0016475 A1
(43) Pub. Date: Jan. 15, 2026

(54) MATERIALS AND METHODS FOR EVALUATION OF ANTIGEN PRESENTATION MACHINERY COMPONENTS AND USES THEREOF

(71) Applicant: Ventana Medical Systems, Inc., Tucson, AZ (US)

(72) Inventors: Birte Aggeler, Tucson, AZ (US); Carolina Dallett, Oakland, CA (US); Lisa Leon Gallegos, Oro Valley, AZ (US); Zhiming Liao, Livermore, CA (US); Dennis Wang, San Ramon, CA (US)

(21) Appl. No.: 19/238,981

(22) Filed: Jun. 16, 2025

Related U.S. Application Data
(63) Continuation of application No. PCT/US2023/085146, filed on Dec. 20, 2023.
(60) Provisional application No. 63/477,010, filed on Dec. 23, 2022.

(51) Int. Cl. G01N 33/574; C07K 16/18

(52) U.S. Cl. CPC G01N 33/57484; C07K 16/18; C07K 2317/565; C07K 2317/567; G01N 2333/70539

(57) ABSTRACT

Disclosed herein are compositions, systems, and methods for identifying subjects who may be responsive to MHC-I-dependent immunotherapeutic agents based upon the expression of the components of the antigen presentation machinery and, in particular, the expression of the constituent elements of the transporter associated with antigen processing complex and the major histocompatibility complex class I.

### CROSS REFERENCE TO RELATED APPLICATION

The present application is a continuation of International Application No. PCT/US2023/085146 filed on Dec. 20, 2023, which applications claims the benefit of the filing date of U.S. Provisional Patent Application No. 63/477,010, filed on Dec. 23, 2022, the disclosures of which are hereby incorporated by reference herein in their entireties.

### SEQUENCE LISTING

The contents of the electronic sequence listing (TAP_MHC_ST26.xml; Size: 22,721 bytes; and Date of Creation: Dec. 20, 2023) is herein incorporated by reference in its entirety.

### FIELD OF THE DISCLOSURE

The present disclosure is directed to assays for evaluating the expression of the constituent elements of the transporter associated with antigen processing complex and/or the expression of the constituent elements of the major histocompatibility complex class I.

### BACKGROUND OF THE DISCLOSURE

Avoidance of the immune system is a critical step in the development of cancer. Recent advances in the manipulation of the immune system have expanded the arsenal available for treating cancer. Class The understanding of immune system avoidance, however, is still lacking. For instance, many tumors develop mechanisms to activate immune checkpoint pathways, which downregulates T-cell responses and creates an immunosuppressed environment. Checkpoint-directed therapies seek to remove this inhibition, thereby seeking to “reactivate” a tumor-directed T-cell response. Several immune checkpoint pathway inhibitors have been approved as cancer treatments by the US Food and Drug Administration, including agents that target Cytotoxic T-Lymphocyte Antigen-4 (CTLA-4), Programmed Death-1 (PD-1), and Programmed Death Ligand 1 (PD-L1). Many more such agents are currently being investigated (See generally Marin-Acevedo et al., Next generation of immune checkpoint inhibitors and beyond, Journal of Hematology and Oncology, 2021, Vol. 14, Art. No. 45). Unfortunately, few patients demonstrate clinically relevant responses to these treatments.

Adequately predictive biomarkers for evaluating responsiveness to checkpoint directed therapy are lacking. PD-L1 is the most widely used biomarker; however, even patients with high PD-L1 levels have relatively low response rates. See Sun. Moreover, objective response rates are highly variable between different indications. Id. The relative number of somatic mutations within a tumor (known as tumor mutational burden or TMB) has also been utilized as a predictive biomarker. See Goodman. Conditions that can lead to high TMB, including mismatch repair deficiency (dMMR) and high microsatellite instability (MSI-H), also have been shown to be predictive of response to checkpoint inhibitors. See Le. However, this predictive value does not hold across all tumors (McGrail et al., High tumor mutation burden fails to predict immune checkpoint blockade response across all cancer types, Annals of Oncology, 2021, Vol. 32, Issue 5, pp. 661-672). In addition, there are tumor types known to respond well to checkpoint inhibitors despite having relatively low TMB values. For example, kidney cancer has among the lowest TMB of all cancers, but a high percentage of these patients respond to immunotherapy. See Yarchoan I.

Other classes of cancer immunotherapies could also benefit from improved biomarkers (see van Belzen & Kesmir (discussing biomarkers for response to adoptive T cell transfer); Hong (reviewing biomarkers for CAR-T therapy); Shindo (reviewing biomarkers used with different immunotherapies); Suekane (reviewing biomarkers associated with response to peptide-based cancer vaccines)).

A need in the art exists for biomarkers that are potentially predictive of response to cancer immunotherapies and methodologies for evaluating such biomarkers in patient samples.

### BRIEF SUMMARY OF THE DISCLOSURE

Disclosed herein are compositions, systems, and methods for identifying subjects who may be responsive to MHC-I-dependent immunotherapeutic agent based upon the expression of the components of the antigen presentation machinery (APM) and, in particular, the expression of the constituent elements of the transporter associated with antigen processing (TAP) complex and the major histocompatibility complex class I (“MHC class I” or “MHC-I”).

In some embodiments, the present disclosure is directed to methods of assessing the expression of the APM within an obtained biological sample, e.g., a histological sample or a cytological sample. In some embodiments, the expression of the APM is assessed by ascertaining the expression of the TAP complex (e.g., by evaluating the expression of either or both the TAP1 and TAP2 proteins) and the MHC-I complex (e.g., by evaluating the expression of HLA-A, HLA-B, and/or HLC-C). In some embodiments, the assessment of the expression of the TAP and MHC-I complexes is made using an immunoenzymatic technique (e.g., immunohistochemistry, immunocytochemistry), flow cytometry, fluorescence-activated cell sorting (FACS) analysis, RNA sequencing (RNA-seq), polymerase chain reaction, enzyme linked immuno-assay (ELISA), etc.

In some embodiments, based on the assessment of the expression of the APM within the obtained biological sample, a subject in need of treatment with an immunotherapeutic agent may be stratified into a first population including those subjects likely to respond to an MHC-I-dependent immunotherapeutic agent; and a second population including those subjects likely not to respond to an MHC-I-dependent immunotherapeutic agent. In other embodiments, based on the assessment of the expression of the APM within the obtained biological sample, suitable candidates for treatment with an MHC-I-dependent immunotherapeutic agent (e.g., a checkpoint inhibitor, a cell-based therapy, a cancer vaccine therapy, etc.) may be identified.

A first aspect of the present disclosure is an affinity histochemical or affinity cytochemical method comprising: (a) contacting a cellular tumor sample with a biomarker-specific reagent under conditions that permit specific binding of the biomarker-specific reagent to the cellular tumor sample, wherein the biomarker-specific agent is one of a HLA-A biomarker specific reagent, a HLA-B biomarker specific reagent, or a HLA-C biomarker specific reagent; (b) removing unbound biomarker-specific reagent from the sample, thereby obtaining a labeled cellular tumor sample; and (c) contacting the labeled cellular tumor sample with a set of detection reagents which interact with the biomarker-specific reagent to facilitate deposition of a detectable moiety on the labeled cellular tumor sample. In some embodiments, the human HLA-A biomarker-specific reagent is a human HLA-A protein biomarker-specific reagent; the human HLA-B biomarker-specific reagent is a human HLA-B protein biomarker-specific reagent; or the human HLA-C biomarker-specific reagent is a human HLA-C protein biomarker-specific reagent. In some embodiments, the human HLA-A protein biomarker-specific reagent is an anti-human HLA-A antibody; the human HLA-B biomarker-specific reagent is a human HLA-B protein biomarker-specific reagent; or the human HLA-C biomarker-specific reagent is a human HLA-C protein biomarker-specific reagent. In some embodiments, the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-C RNA biomarker-specific reagent.

In some embodiments, the cellular tumor sample comprises a tissue section. In some embodiments, the cellular tumor sample comprises a cytology sample. In some embodiments, the detectable moiety is selected from the group consisting of a chromogenic dye, a fluorophore, a mass spectrometer-detectable label, and a nucleic acid barcode.

In some embodiments, the cellular tumor sample is derived from a tumor previously screened by a tumor mutational screen selected from the group consisting of a tumor mutational burden (TMB) screen, a microsatellite stability (MSS) screen, and a tumor previously screened by a mismatch repair (MMR) screen. In some embodiments, the cellular tumor sample is derived from a tumor previously screened for TAP expression.

A second aspect of the present disclosure is an affinity histochemical or affinity cytochemical method comprising: (a) contacting a cellular tumor sample with a human pan-HLA biomarker-specific reagent under conditions that permit specific binding of the biomarker-specific reagent to the cellular tumor sample; (b) removing unbound biomarker-specific reagent from the sample, thereby obtaining a labeled cellular tumor sample; and (c) contacting the labeled cellular tumor sample with a set of detection reagents that interact with the biomarker-specific reagent to facilitate deposition of a detectable moiety on the labeled cellular tumor sample, wherein the human pan-HLA biomarker-specific reagent is a human pan-HLA protein biomarker-specific reagent.

A third aspect of the present disclosure is an affinity histochemical or affinity cytochemical method comprising: (a) contacting one or more cellular tumor samples with a human HLA-A biomarker-specific reagent, a human HLA-B biomarker-specific reagent, and a human HLA-C biomarker-specific reagent under conditions that permit specific binding of the HLA-A, HLA-B, and HLA-C biomarker-specific reagents to the one or more cellular tumor samples; removing unbound HLA-A, HLA-B, and HLA-C biomarker-specific reagent from the one or more cellular tumor samples, thereby obtaining one or more labeled cellular tumor samples; and (c) contacting the one or more labeled cellular tumor samples with a set of detection reagents that interact with the HLA-A, HLA-B, and HLA-C biomarker-specific reagents to facilitate deposition of a detectable moiety on the one or more labeled cellular tumor samples. In some embodiments, the same cellular tumor sample is contacted with the two or more of the human HLA-A, HLA-B, and HLA-C biomarker-specific reagents; or all three of the human HLA-A, HLA-B, and HLA-C biomarker-specific reagents. In some embodiments, different cellular tumor samples are contacted with the human HLA-A, HLA-B, and HLA-C biomarker-specific reagents. In some embodiments, the human HLA-A biomarker-specific reagent is an anti-human HLA-A antibody, the human HLA-B biomarker-specific reagent is an anti-human HLA-B antibody, and the human HLA-C biomarker-specific reagent is an anti-human HLA-C antibody. In some embodiments, the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-C RNA biomarker-specific reagent.

A fourth aspect of the present disclosure is an affinity histochemical or affinity cytochemical method comprising: (a) contacting a cellular tumor sample with a human HLA-A biomarker-specific reagent under conditions that permit specific binding of the biomarker-specific reagent to the cellular tumor sample; (b) contacting the cellular tumor sample with a human HLA-B biomarker-specific reagent under conditions that permit specific binding of the biomarker-specific reagent to the cellular tumor sample; and (c) contacting the cellular tumor sample with a human HLA-C biomarker-specific reagent under conditions that permit specific binding of the biomarker-specific reagent to the cellular tumor sample; wherein the human HLA-A biomarker-specific reagent is conjugated to a first detectable moiety; the human HLA-B biomarker-specific reagent is conjugated to a second detectable moiety; and the human HLA-C biomarker-specific reagent is conjugated to a third detectable moiety. In some embodiments, the method further comprises (d) contacting the cellular tumor sample with a set of detection reagents that interact with the human HLA-A biomarker-specific reagent to facilitate deposition of a first detectable moiety on the cellular tumor sample; (e) contacting the cellular tumor sample with a set of detection reagents that interact with the human HLA-B biomarker-specific reagent to facilitate deposition of a second detectable moiety on the cellular tumor sample; and (f) contacting the cellular tumor sample with a set of detection reagents that interact with the human HLA-C biomarker-specific reagent to facilitate deposition of a third detectable moiety on the cellular tumor sample.

In some embodiments, the human HLA-A biomarker-specific reagent is an anti-human HLA-A antibody, the human HLA-B biomarker-specific reagent is an anti-human HLA-B antibody, and the human HLA-C biomarker-specific reagent is an anti-human HLA-C antibody. In some embodiments, the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-C RNA biomarker-specific reagent.

In some embodiments, the method further comprises contacting the cellular tumor sample with one or more human tumor cell marker biomarker-specific reagents under conditions that permit specific binding of the one or more human tumor cell marker biomarker-specific reagents to the cellular tumor sample. In some embodiments, the method further comprises contacting the cellular tumor sample with a human B2M biomarker-specific reagent under conditions that permit specific binding of the human B2M biomarker-specific reagent to the cellular tumor sample. In some embodiments, the detectable moiety is selected from the group consisting of a chromogenic dye, a fluorophore, a mass spectrometer-detectable label, and a nucleic acid barcode. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen. In some embodiments, the cellular tumor sample is derived from a tumor previously screened for TAP expression.

A fifth aspect of the present disclosure is an affinity histochemical or affinity cytochemical method comprising: (a) contacting a cellular tumor sample with a HLA biomarker-specific reagent under conditions that permit specific binding of the HLA biomarker-specific reagent to the cellular tumor sample; and (b) contacting the cellular tumor sample with a human tumor cell marker biomarker-specific reagent under conditions that permit specific binding of the human tumor cell marker biomarker-specific reagent to the cellular tumor sample; wherein the human HLA biomarker-specific reagent is conjugated to a first detectable moiety and the human tumor cell marker biomarker-specific reagent is conjugated to a second detectable moiety, wherein the first and second detectable moieties are different. In some embodiments, the method further comprises (c) contacting the cellular tumor sample with a set of detection reagents that interact with the human HLA biomarker-specific reagent to facilitate deposition of a first detectable moiety on the cellular tumor sample; and (d) contacting the cellular tumor sample with a set of detection reagents that interact with the tumor cell marker biomarker-specific reagent to facilitate deposition of a second detectable moiety on the cellular tumor sample. In some embodiments, the HLA biomarker-specific reagent is selected from the group consisting of a human HLA-A protein biomarker-specific reagent, a human HLA-B protein biomarker-specific reagent, and a human HLA-C protein biomarker-specific reagent. In some embodiments, the HLA biomarker-specific reagent is a pan-HLA protein biomarker-specific reagent.

In some embodiments, the method further comprises contacting the cellular tumor sample with a human B2M biomarker-specific reagent under conditions that permit specific binding of the human B2M biomarker-specific reagent to the cellular tumor sample. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen.

A sixth aspect of the present disclosure is an affinity histochemical or affinity cytochemical method comprising: (a) contacting a cellular tumor sample with one or more human biomarker-specific reagents under conditions that permit specific binding of the one or more human biomarker-specific reagents to the cellular tumor sample, wherein the one or more human biomarker-specific reagents are selected from the group consisting of a human TAP1 biomarker specific reagent and a human TAP2 biomarker specific reagent; (b) removing unbound one or more human biomarker-specific reagents from the cellular tumor sample, thereby obtaining a labeled cellular tumor sample; and (c) contacting the labeled cellular tumor sample with one or more sets of detection reagents that interact with the one or more human biomarker-specific reagents to facilitate deposition of a detectable moiety on the labeled cellular tumor sample. In some embodiments, the human TAP1 biomarker-specific reagent is a human TAP1 protein biomarker-specific reagent; or the human TAP2 biomarker-specific reagent is a human TAP2 protein biomarker-specific reagent. In some embodiments, the human TAP1 protein biomarker-specific reagent is an anti-human TAPI antibody; or the human TAP2 protein biomarker-specific reagent is an anti-human TAP2 antibody. In some embodiments, the cellular tumor sample is contacted with both the human TAP1 biomarker specific reagent and the human TAP21 biomarker specific reagent. In some embodiments, the human TAP1 biomarker-specific reagent and the human TAP2 biomarker-specific reagent are applied separately. In some embodiments, the human TAP1 biomarker-specific reagent and a human TAP2 biomarker-specific reagent are applied via a human pan-TAP biomarker-specific reagent cocktail

In some embodiments, the cellular tumor sample is a tissue section. In some embodiments, the cellular tumor sample is a cytology sample. In some embodiments, the detectable moiety is selected from the group consisting of a chromogenic dye, a fluorophore, a mass spectrometer-detectable label, and a nucleic acid barcode. In some embodiments, the cellular tumor sample is derived from a tumor previously determined to express one or more of HLA-A, HLA-B, or HLA-C. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen.

A seventh aspect of the present disclosure is an affinity histochemical or affinity cytochemical method comprising: (a) contacting a first cellular tumor sample with a human TAP1 biomarker-specific reagent under conditions that permit specific binding of the biomarker-specific reagent to the first cellular tumor sample; (b) removing unbound biomarker-specific reagent from the first cellular sample, thereby obtaining a first labeled cellular tumor sample; (c) contacting a second cellular tumor sample with a human TAP2 biomarker-specific reagent under conditions that permit specific binding of the biomarker-specific reagent to the second cellular tumor sample; and (d) removing unbound biomarker-specific reagent from the second cellular tumor sample, thereby obtaining a second labeled cellular tumor sample. In some embodiments, the method further comprises (e) contacting the first labeled cellular tumor sample with a set of detection reagents that interact with the biomarker-specific reagent to facilitate deposition of a detectable moiety on the first labeled cellular tumor sample; and (f) contacting the second labeled cellular tumor sample with a set of detection reagents that interact with the biomarker-specific reagent to facilitate deposition of a detectable moiety on the second labeled cellular tumor sample. In some embodiments, the human TAP1 biomarker-specific reagent is a human TAP1 protein biomarker-specific reagent; or the human TAP2 biomarker-specific reagent is a human TAP2 protein biomarker-specific reagent. In some embodiments, the human TAP1 protein biomarker-specific reagent is an anti-human TAP1 antibody; or the human TAP2 protein biomarker-specific reagent is an anti-human TAP2 antibody. In some embodiments, the human TAP1 biomarker-specific reagent is a human TAP1 RNA biomarker-specific reagent and the human TAP2 biomarker-specific reagent is a human TAP2 RNA biomarker-specific reagent. In some embodiments, the first and second cellular tumor samples are tissue sections. In some embodiments, the first and second cellular tumor samples are cytology samples. In some embodiments, the first and second cellular tumor samples are derived from a tumor previously determined to express one or more of HLA-A, HLA-B, or HLA-C. In some embodiments, the first and second cellular tumor samples are derived from a tumor previously screened by a tumor mutational screen selected from the group consisting of a tumor mutational burden screen, a microsatellite stability screen, and a tumor previously screened by a mismatch repair screen.

An eighth aspect of the present disclosure is an affinity histochemical or affinity cytochemical method comprising: (a) contacting a cellular tumor sample with a human TAP1 biomarker-specific reagent under conditions that permit specific binding of the biomarker-specific reagent to the cellular tumor sample; and (b) contacting the cellular tumor sample with a human TAP2 biomarker-specific reagent under conditions that permit specific binding of the biomarker-specific reagent to the cellular tumor sample; wherein the human TAP1 biomarker-specific reagent is conjugated to a first detectable moiety; and the human TAP2 biomarker-specific reagent is conjugated to a second detectable moiety. In some embodiments, the method further comprises (c) contacting the cellular tumor sample with a set of detection reagents that interact with the human TAP1 biomarker-specific reagent to facilitate deposition of a first detectable moiety on the cellular tumor sample; and (d) contacting the cellular tumor sample with a set of detection reagents that interact with the human TAP2 biomarker-specific reagent to facilitate deposition of a second detectable moiety on the cellular tumor sample. In some embodiments, the human TAP1 biomarker-specific reagent is a human TAP1 protein biomarker-specific reagent; or the human TAP2 biomarker-specific reagent is a human TAP2 protein biomarker-specific reagent. In some embodiments, the human TAP1 protein biomarker-specific reagent is an anti-human TAPI antibody; or the human TAP2 protein biomarker-specific reagent is an anti-human TAP2 antibody. In some embodiments, the detectable moiety is selected from the group consisting of a chromogenic dye, a fluorophore, a mass spectrometer-detectable label, and a nucleic acid barcode. In some embodiments, the cellular tumor sample is derived from a tumor previously determined to express one or more of HLA-A, HLA-B, or HLA-C. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen.

A ninth aspect of the present disclosure is an affinity histochemical or affinity cytochemical method comprising: (a) contacting a cellular tumor sample with a human TAP biomarker-specific reagent under conditions that permit specific binding of the human TAP biomarker-specific reagent to the cellular tumor sample; and (b) contacting the cellular tumor sample with a tumor cell marker biomarker-specific reagent under conditions that permit specific binding of the human tumor cell marker biomarker-specific reagent to the cellular tumor sample; wherein the human TAP biomarker-specific reagent is conjugated to a first detectable moiety and the human tumor cell marker biomarker-specific reagent is conjugated to a second detectable moiety, wherein the first and second detectable moieties are different. In some embodiments, the method further comprises (c) contacting the cellular tumor sample with a set of detection reagents that interact with the human TAP biomarker-specific reagent to facilitate deposition of a first detectable moiety on the cellular tumor sample; and (d) contacting the cellular tumor sample with a set of detection reagents that interact with the tumor cell marker biomarker-specific reagent to facilitate deposition of a second detectable moiety on the cellular tumor sample. In some embodiments, the human TAP biomarker-specific reagent is an anti-human TAPI antibody or an anti-human TAP2 antibody. In some embodiments, the human TAP biomarker-specific reagent is a human pan-TAP protein biomarker-specific reagent. In some embodiments, the cellular tumor sample is derived from an epithelial tumor and the human tumor cell biomarker-specific reagent is a human cytokeratin biomarker-specific reagent. In some embodiments, the human cytokeratin biomarker-specific reagent is a pan-cytokeratin antibody cocktail. In some embodiments, the cellular tumor sample is derived from a mesenchymal tumor and the human tumor cell marker biomarker-specific reagent is a vimentin biomarker-specific reagent. In some embodiments, the cellular tumor sample is derived from a tumor previously determined to express one or more of HLA-A, HLA-B, or HLA-C. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen. In some embodiments, the cellular tumor sample is derived from a tumor of lymphoid origin and the human tumor cell marker biomarker-specific reagent is a CD45 biomarker-specific reagent.

A tenth aspect of the present disclosure is an affinity histochemical or affinity cytochemical method comprising: (a) contacting a cellular tumor sample with a human TAP biomarker-specific reagent under conditions that permit specific binding of the human TAP biomarker-specific reagent to the cellular tumor sample; and (b) contacting the cellular tumor sample with either or both of a human HLA biomarker-specific reagent and/or a human B2M biomarker-specific reagent under conditions that permit specific binding of the human HLA and/or human B2M biomarker-specific reagents to the cellular tumor sample. In some embodiments, the human TAP biomarker-specific reagent is conjugated to a first detectable moiety; and the human HLA biomarker-specific reagent is conjugated to a second detectable moiety, wherein the first and second detectable moieties are different. In some embodiments, the method further comprises: (c) contacting the cellular tumor sample with a set of detection reagents that interact with the human TAP biomarker-specific reagent to facilitate deposition of a first detectable moiety on the cellular tumor sample; and (d) contacting the cellular tumor sample with a set of detection reagents that interact with the HLA biomarker-specific reagent and/or the human B2M biomarker-specific reagent to facilitate deposition of a second detectable moiety on the cellular tumor sample. In some embodiments, the human TAP biomarker-specific reagent is an anti-human TAP1 antibody or an anti-human TAP2 antibody.

In some embodiments, the human TAP biomarker-specific reagent is a human pan-TAP protein biomarker-specific reagent. In some embodiments, the human HLA biomarker-specific reagent is selected from the group consisting of a human HLA-A biomarker-specific reagent, a human HLA-B biomarker-specific reagent, and a human HLA-C biomarker-specific reagent. In some embodiments, the human HLA biomarker-specific reagent is a human pan-HLA protein biomarker-specific reagent. In some embodiments, the cellular tumor sample is contacted with both a human TAP1 biomarker-specific reagent and a human TAP2 biomarker-specific reagent, wherein the human TAP1 and TAP2 biomarker-specific reagents are each conjugated to different detectable moieties. In some embodiments, the cellular tumor sample is derived from a tumor previously screened by a tumor mutational screen selected from the group consisting of a tumor mutational burden screen, a microsatellite stability screen, and a tumor previously screened by a mismatch repair screen.

An eleventh aspect of the present disclosure is a 2-plex chromogenic immunohistochemical assay comprising: (a) contacting a first tissue section derived from tumor with an anti-human TAP monoclonal antibody under conditions that permit specific binding of the anti-human TAP monoclonal antibody to the first tissue section; (b) contacting the first tissue section with a set of detection reagents that interact with the anti-human TAP monoclonal antibody bound to the tissue section to chromogenically deposit a first brightfield dye on the tissue section; (c) contacting a second tissue section derived from the tumor with either an anti-human HLA monoclonal antibody or an anti-human B2M monoclonal antibody under conditions that permit specific binding of the anti-human HLA monoclonal antibody or the anti-human B2M monoclonal antibody to the second tissue section; and (d) contacting the second tissue section with a set of detection reagents that interact with the anti-human HLA monoclonal antibody or the anti-human B2M monoclonal antibody bound to the second tissue section to chromogenically deposit a second brightfield dye on the second tissue section. In some embodiments, the first and second brightfield dyes are separately detectable on the tissue sections. In some embodiments, the human TAP biomarker-specific reagent is an anti-human TAPI antibody or an anti-human TAP2 antibody. In some embodiments, the human TAP biomarker-specific reagent is a human pan-TAP protein biomarker-specific reagent. In some embodiments, the human HLA biomarker-specific reagent is selected from the group consisting of a human HLA-A biomarker-specific reagent, a human HLA-B biomarker-specific reagent, and a human HLA-C biomarker-specific reagent. In some embodiments, the human HLA biomarker-specific reagent is a human pan-HLA protein biomarker-specific reagent.

A twelfth aspect of the present disclosure is a method of quantifying a percentage of TAP biomarker positive tumors cells and a percentage of TAP biomarker positive immune cells in a cellular tumor sample comprising: (a) staining dissociated cells within a first aliquot of the cellular tumor sample for the presence of the TAP biomarker and a tumor cell biomarker; (b) staining dissociated cells within a second aliquot of the cellular tumor sample for the presence of the TAP biomarker and an immune biomarker; (c) obtaining fluorescence data for the stained dissociated cells within each of the first and second aliquots; (d) identifying, based on the obtained fluorescence data, a TAP biomarker positive tumor cell population within the first aliquot and a TAP biomarker positive immune cell population in the second aliquot; and (e) quantifying the percentage of TAP biomarker positive tumor cells and the percentage of TAP biomarker positive immune cells within the cellular tumor sample. In some embodiments, the tumor cell biomarker is an epithelial marker. In some embodiments, the epithelial marker is a cytokeratin. In some embodiments, the cytokeratin is one of a specific cytokeratin marker or pan-cytokeratin. In some embodiments, the immune cell biomarker is selected from the group consisting of CD45, CD3, CD4, CD8, CD20, CD 25, CD19, CD163, CD68, CD69 and CD103. In some embodiments, the obtained fluorescence data comprises scatter plots of fluorescence intensity versus side scatter content. In some embodiments, the identifying of the TAP biomarker positive tumor cell population comprises performing a first sequential gating operation on the obtained fluorescence data for the stained dissociated cells within the first aliquot; and wherein the identifying of the TAP biomarker positive immune cell population comprises performing a second sequential gating operation on the obtained fluorescence data for the stained dissociated cells within the second aliquot.

### BRIEF DESCRIPTION OF THE FIGURES

The patent or application file contains at least one drawing executed in color. Copies of this patent or patent application publication with color drawings will be provided to the Office upon request and the payment of the necessary fee.

FIG. 1A illustrates a workflow for stratifying patients who likely may respond to an MHC-I-dependent immunotherapeutic agent from those patients who are likely not to respond to an MHC-I-dependent immunotherapeutic agent. In particular, FIG. 1A illustrates a method in which an MHC screen is first performed on a cellular tumor sample. If the cellular tumor sample is assessed to be MHC(−), no further evaluation of the cellular tumor sample is performed. If, however, the tumor is assessed to be MHC(+), a TAP status is evaluated. If the cellular tumor sample is evaluated to be TAP(−), no mutational screen is necessary, as these tumors are likely to be immunogenic regardless of the mutational status. If the tumor is assessed to be TAP(+), a mutational screen is performed, as the immunogenicity of these tumors is likely to be highly dependent on the mutational status.

FIG. 1B illustrates an alternative method of stratifying likely responders of an MHC-I-dependent immunotherapeutic agent from likely non-responders. FIG. 1B illustrates a method in which a TAP screen is performed first. If the tumor is assessed to be TAP(−), then there is no need for a mutational screen. If the tumor is TAP(+), an MHC screen is evaluated. If the tumor is MHC(−), no mutational screen is necessary, as these tumors are likely to be immunogenic regardless of the mutational status. If the tumor is MHC(+), a mutational screen is performed, as the immunogenicity of these tumors is likely to be highly dependent on the mutational status.

FIG. 1C illustrates another alternative method of stratifying likely responders of an MHC-I-dependent immunotherapeutic agent from likely non-responders. FIG. 1C illustrates a method in which an MHC-I screen, and a TAP screen are conducted simultaneously. A mutational screen is only performed if the tumor is assessed as MHC(+)/TAP(+), as the immunogenicity of these tumors is likely to depend on the tumor's mutational status. In all other cases, the tumor is likely to be immunogenic (MHC+/TAP−) or is unlikely to be immunogenic (MHC−/TAP+ and MHC−/TAP−) regardless of mutational screen status.

FIG. 2A depicts a method of evaluating a patient's cellular tumor sample and selecting a course of treatment based on the evaluation. Here, an MHC screen is first conducted. If the patient is evaluated as MHC(+), TAP status is used to determine whether the patient will need a mutational screen to select a therapeutic approach. If the status of the cellular tumor sample is TAP(−), the subject may receive an MHC-I-dependent immunotherapeutic agent. If the status of the cellular tumor sample is TAP(+), a mutational screen will be used to select the immunotherapy.

FIG. 2B depicts a method of evaluating a patient's cellular tumor sample and selecting a course of treatment. Here, MHC and TAP screens are conducted simultaneously, such as in a duplex format assay. If the sample is MHC(+), TAP status of the cellular tumor sample is used to determine whether the patient will need a mutational screen to select the therapeutic approach. If the status of the cellular tumor sample is TAP(−), the subject may receive an MHC-I-dependent immunotherapeutic agent. If the status of the cellular tumor sample is TAP(+), a mutational screen will be used to select the immunotherapy.

FIG. 2C depicts a method of evaluating a patient's cellular tumor sample and selecting a course of treatment. FIG. 2C illustrates a workflow in which a TAP screen is used to evaluate patients who would otherwise not be eligible for MHC-I-dependent immunotherapies on the basis of a mutation screen. In this workflow, patients having a cellular tumor sample determined to be MHC(+)/pMMR/MSI-L/TMB-L are screened for TAP expression. Patients having a TAP (−) cellular tumor sample are selected to receive the MHC-I-dependent immunotherapeutic agent, while TAP(+) patients are referred to an alternate therapy.

FIGS. 2D and 2E illustrate workflows in which MHC and TAP screens are both used to stratify patients following a mutational screen. As set forth in FIG. 2D, if a cellular tumor sample classified as dMMR/MSI-H/TMB-H is determined to be MHC(+), the patient is administered the MHC-I-dependent immunotherapeutic agent. With reference to FIG. 2E, if a patient's cellular tumor sample is classified as pMMR/MSI-L/TMB-L, a TAP screen is performed. If the cellular tumor sample is TAP(+), then an alternate therapy is selected. If the cellular tumor sample is assessed to be TAP(−), then an MHC screen is performed. If the TAP(+) cellular tumor sample is determined to be MHC(−), then an alternate therapy is selected. If the TAP(+) cellular tumor sample is determined to be MHC(+), then the patient is treated with the MHC-I-dependent immunotherapy.

FIG. 2F illustrates a workflow in which a combined MHC/TAP status is used to stratify patients following a mutational screen. For cellular tumor samples classified as dMMR/MSI-H/TMB-H, patients are administered the MHC-I-dependent immunotherapeutic agent if the tumor is determined to be MHC(+)/TAP(−) or MHC(+)/TAP(+). For pMMR/MSI-L/TMB-L tumors, patients are administered the MHC-I-dependent immunotherapeutic agent if the tumor is determined to be MHC(+)/TAP(−). All other patients are referred to an alternate therapy.

FIG. 3 illustrates how imbalances in antigen presentation machinery expression may lead to the presentation of T-cell epitopes associated with impaired peptide processing (TEIPPs), which are neoantigens described from alternatively processed self-peptides.

FIG. 4A depicts cell line blocks that were made from control 293T cells and, in particular, 293T cells expressing TAP1 (293T-TAP1), and 293T cells expressing TAP2 (293T-TAP2). Sections from the cell blocks were stained with an anti-TAP1 antibody. The TAP1 antibody only recognizes the cell line over-expressing TAP1.

FIG. 4B illustrates tonsil tissue stained for TAP1 as a positive control.

FIGS. 5A-5C illustrate the variability in TAPI expression by immunohistochemistry in tumor samples from kidney cancer (FIG. 5A), breast cancer (FIG. 5B), and bladder cancer (FIG. 5C). As depicted, the immune cells consistently stain positive for TAP1 even when TAP1 expression in tumor cells is low or absent.

FIG. 6 shows a western blot of lysates from positive control cell lines expressing TAP1 (293T TAP1, HDLM2, and U266B1) and negative control cell lines not expressing TAP1 (293T-TAP2, RPMI6226, and 293T).

FIG. 7A presents an examination of antigen presentation machinery expression in kidney tumor cases by immunohistochemistry staining of a kidney tumor microarray. Specifically, FIG. 7A provides examples of TAP1 and HLA-A staining in three ccRCC cases showing (1) normal (intact) expression of both TAP1 and HLA-A; (2) loss of expression of TAP1 and HLA-A; and (3) TAP1 loss with normal (intact) expression of HLA-A.

FIG. 7B provides a summary table of TAP1 and HLA-A status across ccRCC samples on the kidney tumor microarray (see FIG. 7A).

FIG. 8A presents an examination of antigen presentation machinery expression across 94 lung cancer cases on a tumor microarray. Specifically, FIG. 8A shows examples of TAP1 and HLA-A staining in three cases showing (1) normal (intact) expression of both TAP1 and HLA-A; (2) loss of expression of TAP1 and HLA-A; and (3) TAP1 loss with normal (intact) expression of HLA-A.

FIG. 8B provides a summary table of TAP1 and HLA-A status across the lung tumor microarray (see FIG. 8A).

FIG. 9 demonstrates the bulk RNA expression of antigen presentation machinery components in ccRCC patients and outcomes on MHC-I-dependent immunotherapeutic agent. Lower expression of TAP1 is significantly associated with survival on MHC-I-dependent immunotherapeutic agent.

FIG. 10 shows TAP1 immunohistochemistry staining in ccRCC tissues and provides examples of TAP1 scoring. Panel (a) shows intact TAP1 staining, with all cells in the tumor and tumor microenvironment staining positive. Panel (b) illustrates heterogeneous TAP1 staining in tumor cells, with some tumor cells losing expression of TAP1 and others retaining expression. Note staining of 2+ or greater in normal cells in the tumor microenvironment. Panel (c) shows loss of TAP1 staining in tumor cells. Note staining of 2+ or greater in normal cells in the tumor microenvironment. Panel (d) sets forth an example of ambiguous staining, with loss of TAP1 in the tumor cells, but also in the normal cells of the tumor microenvironment. In these cases, loss of TAP1 could be due to unfavorable preanalytic variables. These samples would not be scored for TAP1.

FIG. 11 illustrates a method of conducting an RNAseq analysis in accordance with one embodiment of the present disclosure (Kukurba K R, Montgomery S B. RNA Sequencing and Analysis. Cold Spring Harb Protoc. 2015 Apr. 13; 2015 (11): 951-69. doi: 10.1101/pdb.top084970. PMID: 25870306; PMCID: PMC4863231).

FIGS. 12A and 12B depict methods of assessing the percentage of TAP positive tumor cells and TAP positive immune cells in a cellular tumor sample using flow cytometry.

FIG. 13 illustrates a method of preparing control aliquots and tumor marker aliquots for flow cytometry analysis.

FIGS. 14A and 14B provide methods of preparing tumor marker aliquots (FIG. 14A), immune marker aliquots (FIG. 14B), and a plurality of control aliquots (FIGS. 14A and 14B).

FIGS. 15A and 15B provide scatter plots of obtained flow cytometry data. In particular, FIGS. 15A and 15B depict the flow cytometric quantification of TAP1 in tumor and immune populations from cells dissociated from homogenized formalin fixed tumor tissue of a ccRCC patient. In particular, in FIG. 15A the data represented as black dots on the right scatter plot was derived from a “double stained sample” that was stained for the tumor marker Cytokeratin 8/18 (CK8/18) using Alexa Fluor 488 and TAPI using Alexa Fluor 647. The samples were also stained for DAPI for doublet discrimination (not shown). The left scatter plot shows CK8/18 staining. The data represented as a red overlay was derived from an aliquot of unstained cells from the same case and was used as a reference for gating the CK8/18 positive (CK8/18+) cells. The cell population surrounded by the blue gate is CK8/18+. The right scatter plot shows gating of the “double stained” sample for TAP1 positive cells (green gate) within the CK8/18+ gated population from the plot on the left. The data represented as a blue overlay are from an aliquot of cells from the same case, stained only for CK8/18, and gated for CK8/18+ cells. These cells were not stained for TAP1 and served as a negative control reference to assist with gating the TAP1+ population from the “double stained” sample. The data in FIG. 15B is similar to the data in FIG. 15A except the tumor marker CK8/18 is replaced with an immune cell marker, namely CD45.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal, or mucosal route of administration, for example, intranasally, vaginally, rectally, sublingually, or topically. Administering can also be performed, for example, once, a plurality of times, and/or over one or more extended periods.

As used herein, the terms “amount” or “level” of a biomarker is a detectable level or amount a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment.

As used herein, the term “antibody” refers to and encompasses various antibody structures, including but not limited to monoclonal antibodies, polyclonal antibodies, multispecific antibodies (e.g., bispecific antibodies), and antibody fragments so long as they exhibit the desired antigen-binding activity.

As used herein, the term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

As used herein, the phrase “antigen presentation” refers to the process by which cells in the body display antigens on their cell surfaces in a form recognizable by lymphocytes.

As use herein, the terms “binds,” “specific binding,” “specifically binds to” or is “specific for” refers to measurable and reproducible interactions such as binding between a target and a specific binding agent, which is determinative of the presence of the target in the presence of a heterogeneous population of molecules including biological molecules. For example, a binding entity that specifically binds to a target may be an antibody that binds the target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “biomarker” shall refer to any molecule or group of molecules found in a biological sample that can be used to characterize the biological sample or a subject from which the biological sample is obtained. For example, a biomarker may be a molecule or group of molecules whose presence, absence, or relative abundance is characteristic of a particular cell or tissue type or state; or characteristic of a particular pathological condition or state; or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof.

As used herein, the phrase “biomarker specific reagent” refers to a specific detection reagent that is capable of specifically binding directly to one or more biomarkers in the cellular sample, such as a primary antibody.

As used herein, the phrase “specific detection reagent” refers to a composition of matter that is capable of specifically binding to a target chemical structure in the context of a cellular sample. Exemplary specific detection reagents include nucleic acid probes specific for particular nucleotide sequences; antibodies and antigen binding fragments thereof; and engineered specific binding compositions, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, CA), dAbs (scaffold based on VH or VL antibody domain; GlaxoSmithKline PLC, Cambridge, UK), DARPins (scaffold based on Ankyrin repeat proteins; Molecular Partners AG, Zürich, CH), ANTICALINs (scaffold based on lipocalins; Pieris AG, Freising, DE), NANOBODYs (scaffold based on VHH (camelid Ig); Ablynx N/V, Ghent, BE), TRANS-BODYs (scaffold based on Transferrin; Pfizer Inc., New York, NY), SMIPs (Emergent Biosolutions, Inc., Rockville, MD), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference.

As used herein, the term “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. The term “cancer” is generally used interchangeably with “tumor” herein (unless a tumor is specifically referred to as a “benign” tumor, which is an abnormal mass of cells that lacks the ability to invade neighboring tissue or metastasize), and encompasses malignant solid tumors (e.g., carcinomas, sarcomas) and malignant growths in which there may be no detectable solid tumor mass (e.g., certain hematologic malignancies). Non-limiting examples of cancers include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phacomatoses, edema (such as that associated with brain tumors), Meigs' syndrome, brain, as well as head and neck cancer, and associated metastases. In certain embodiments, cancers that are amenable to treatment by the antibodies of the present disclosure include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma, or metastatic lung cancer.

As used herein, the term “cellular sample” refers to any sample containing intact cells, such as cell cultures, bodily fluid samples or surgical specimens taken for pathological, histological, or cytological interpretation.

As used herein, the terms “chromogen” or “chromogenic compound” and the like refer to a substance that can be converted into a colored compound under specific conditions, e.g., when acted upon by an enzyme or under specific chemical/reaction conditions. Examples of enzyme-substrate combinations include: (i) Horseradish peroxidase (HRP) with hydrogen peroxidase as a substrate, where the hydrogen peroxidase oxidizes a dye precursor [e.g., orthophenylene diamine (OPD) or 3,3′,5,5′-tetramethyl benzidine hydrochloride (TMB)]; (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). Numerous other enzyme-substrate combinations are available to those skilled in the art. For a general review of these, see U.S. Pat. Nos. 4,275,149 and 4,318,980.

As used herein, the term “cytological sample” refers to a cellular sample that either have no cross-sectional spatial relationship in vivo (such as cellular samples derived from blood samples, urine samples, sputum, etc.) or in which the cross-sectional spatial relationship has been at least partially disrupted (such as tissue smears, liquid-based cytology samples, fine needle aspirates, etc.).

As used herein, a “detectable moiety” refers to a molecule or material that can produce a detectable signal (such as visually, electronically, or otherwise) that indicates the presence (i.e., qualitative analysis) and/or concentration (i.e., quantitative analysis) of the detectable moiety deposited on a sample. The term “detectable moiety” includes, but is not limited to, chromogenic, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity), and moieties compatible with mass cytometry imaging (such as multiplexed ion beam imaging (“MIBI,” described at Baharlou, Bodenmiller, and Ptacek) or Imaging Mass Cytometry (“IMB,” described by Baharlou and Bodenmiller)). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11th Edition. In other embodiments, the detectable moiety is a molecule detectable via brightfield microscopy, such as dyes including diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine). In yet other embodiments, the detectable moiety is compatible with mass cytometry imaging, such as a stable metal isotope (including but lanthanide series metals).

As used herein, the term “detection reagent” refers to any reagent used to deposit a detectable moiety in proximity to a biomarker-specific reagent bound to a biomarker in a cellular sample to thereby stain the sample. Non-limiting examples include secondary detection reagents (such as secondary antibodies capable of binding to a primary antibody, anything that specifically binds biotin or avidin), tertiary detection reagents (such as tertiary antibodies capable of binding to secondary antibodies), enzymes directly or indirectly associated with the specific binding agent, chemicals reactive with such enzymes to effect deposition of a fluorescent or chromogenic stain, wash reagents used between staining steps, and the like.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the phrase “immune checkpoint molecule” refers to a protein expressed by an immune cell whose activation down-regulates a cytotoxic T-cell response. Examples include PD-1, TIM-3, LAG-4, and CTLA-4.

As used herein, the phrase “immune escape biomarker” refers to a biomarker expressed by a tumor cell that helps the tumor avoid a T-cell mediated immune response. Examples of immune escape biomarkers include PD-L1, PD-L2, and IDO.

As used herein, the phrase “cell-based immunotherapeutic” refers to an isolated preparations of immune cells suitable for administration to a patient having a tumor. Examples include autologous T-cells, engineered T-cell receptors T-cells (eTCR-T), and chimeric antigen receptor T-cell (CAR-T).

As used herein, the phrase “autologous T-cells” refers to T-cells obtained from a patient that are expanded and then administered to the patient. In some instances, specific populations of T-cells are enriched, expanded, and then administered to the patients.

As used herein, the phrase “engineered T-cell Receptor T-cells (eTCR-T)” refers to T-cells engineered ex vivo to express a T-cell receptor to a specific antigen-MHC-I complex. Examples of eTCR-Ts are reviewed by Zhao & Cao, Engineered T Cell Therapy for Cancer in the Clinic, Frontiers in Immunology, 2019, 10:2250. doi 10.3389/fimmu.2019.02250.

As used herein, the term “MHC” (+)” refers to a tumor in which the extent of MHC loss is equal to or less than a stratification cutoff.

As used herein, the term “MHC” (−)” refers to a tumor in which the extent of MHC loss exceeds the stratification cutoff.

As used herein, the terms “MHC-I-dependent immunotherapeutic agent” or “MHC-I-dependent immunotherapies” refer to any therapy in which one or more substance(s) are used to provoke, restore, enhance, stimulate, increase, or adjust an immune response against a tumor or cancer cells, wherein the immune response is dependent at least in part on presentation of MHC-I-ligands by the tumor. Exemplary MHC-I-dependent immunotherapeutic agents include immune checkpoint-directed therapies, T-cell directed bispecific therapies, cell-based MHC-I-dependent immunotherapies, and cancer vaccine therapies.

As used herein, the term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic, or other techniques known to those skilled in the art.

As used herein, the term “tumor neoantigen” refers to an antigen produced by a tumor cell. Exemplary tumor neoantigens include antigenic fragments of polypeptides arising from somatic mutations, such as single nucleotide mutations, gene fusions, intron retention, insertions, and deletions; oncogenic viral particles; tumor-associated post-translational modifications, and novel peptides resulting from loss of functional TAP complex. See Zhu & Liu, Marjit.

As used herein, the term “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, the terms “primary antibody” and “secondary antibody” refer to different antibodies, where a primary antibody is a polyclonal or monoclonal antibody from one species (rabbit, mouse, goat, donkey, etc.) that specifically recognizes an antigen (e.g., a biomarker) in a sample (e.g., a human biological sample) under study, and a secondary antibody is an antibody (usually polyclonal) from a different species that specifically recognizes the primary antibody, e.g., in its Fc region.

As used herein, the term “sample” shall refer to any material obtained from a subject capable of being tested for the presence or absence of a biomarker.

As used herein, the term “section” refers to a thin slice of a tissue sample suitable for microscopic analysis, typically cut using a microtome. When used as a verb, the process of generating a section.

As used herein, the term “serial section” shall refer to any one of a series of sections cut in sequence by a microtome from a tissue sample. For two sections to be considered “serial sections” of one another, they do not necessarily need to be consecutive sections from the tissue, but they should generally contain sufficiently similar tissue structures in the same spatial relationship, such that the structures can be matched to one another after histological staining.

As used herein, the term “slide” refers to any substrate (e.g., substrates made, in whole or in part, glass, quartz, plastic, silicon, etc.) of any suitable dimensions on which a cellular sample is placed for analysis, and for example, a “microscope slide” such as a standard 3 inch by 1 inch microscope slide or a standard 75 mm by 25 mm microscope slide.

As used herein, the phrases “specific binding agent” or “specific binding entity” refer to any composition of matter that is capable of specifically binding to a target chemical structure associated with a cellular sample (such as a biomarker expressed by the sample, or a biomarker-specific reagent bound to the sample). Examples include but are not limited to nucleic acid probes specific for particular nucleotide sequences; antibodies and antigen binding fragments thereof; and engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, CA), dAbs (scaffold based on VH or VL antibody domain; GlaxoSmithKline PLC, Cambridge, UK), DARPins (scaffold based on Ankyrin repeat proteins; Molecular Partners AG, Zurich, CH), ANTICALINs (scaffold based on lipocalins; Pieris AG, Freising, DE), NANOBODYS (scaffold based on VHH (camelid Ig); Ablynx N/V, Ghent, BE), TRANS-BODYs (scaffold based on Transferrin; Pfizer Inc., New York, NY), SMIPs (Emergent Biosolutions, Inc., Rockville, MD), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on DISCOVERY Research and Clinical Validation, Current Pharmaceutical Biotechnology, Vol. 9, pp. 502-509 (2008), the content of which is incorporated by reference.

When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

As used herein, the term “TAP(+)” refers to a tumor in which the extent of TAP loss is less than a stratification cutoff.

As used herein, the term “TAP(−)” refers to a tumor in which the extent of TAP loss exceeds a stratification cutoff.

As used herein, the term “tumor” refers to all neoplastic cell growth and proliferation, whether malignant or benign, and all pre-cancerous and cancerous cells and tissues. In some embodiments, the tumor is a malignant cancerous tumor (i.e., cancer). In some embodiments, the tumor is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors.

As used herein, the phrase “tumor mutational screening” refers to any methodology of classifying a tumor on the basis of the relative quantity of one or more classes of mutations within the tumor genome and/or dysfunction in one of more DNA repair pathways in the tumor. Exemplary tumor mutational screening methodologies include tumor mutational burden (TMB) screening, microsatellite instability (MSI) screening, and mismatch repair (MMR) screening.

As used herein, the phrase “tumor mutational burden screening” and “TMB screening” refers to any methodology of classifying a tumor on the basis of a quantification or estimation of somatic mutations within a tumor genome. Exemplary methods and systems for evaluating TMB (and determining a TMB status of a sample) are described in Melendez et al., Methods of measurement for tumor mutational burden in tumor tissue, Translational Lung Cancer Research, 2018, Vol. 7, Issue 6, pp. 661-667; Heydt et al., Analysis of tumor mutational burden: correlation of five large gene panels with whole exome sequencing, Scientific Reports, 2020, 10:11387; Yao et al., ecTMB: a robust method to estimate and classify tumor mutational burden, Scientific Reports, 2020, Vol. 10, Art. No. 4983; Tian et al., A novel tumor mutational burden estimation model as a predictive and prognostic biomarker in NSCLC patients, BMC Medicine, 2020, Vol. 18, Art. No. 232; United States Patent Publication Nos. 2018/0363066 and 2020/0258601 and PCT Publication No. WO/2020/136133, each of which is hereby incorporated by reference herein in its entirety.

As used herein, the phrase “microsatellite instability screening” or “MSI screening” refers to any methodology of classifying a tumor on the basis of the accumulation of alterations in the length of microsatellite loci. Exemplary classification include microsatellite instability-high (“MSI-H”) tumors, in which alterations in the length of microsatellite loci have accumulated in the tumor beyond a pre-determined threshold; and microsatellite instability-low (“MSI-L”) tumors, in which alterations in the length of microsatellite loci have not accumulated beyond the pre-determined threshold. Exemplary methodologies for evaluating MSI status are disclosed at, for example, Murphy et al., J. Mol. Diagn., Vol. 8, Issue 3, pp. 305-11 (July 2006); Esemuede et al., Ann. Surg. Oncol., vol. 17, Issue 12, pp. 3370-78 (December 2010); Mukherjee et al., Hereditary Cancer in Clinical Practice, Vol. 8, Issue 9 (2010); MSI Analysis System (Promega) (evaluation of seven markers for MSI phenotype, including five nearly monomorphic mononucleotide repeat markers (BAT-25, BAT-26, MONO-27, NR-21 and NR-24) and two highly polymorphic pentanucleotide repeat markers (Penta C and Penta D)), each of which is hereby incorporated by reference herein in its entirety.

As used herein a “mismatch repair screen” shall refer to any methodology for evaluating the expression level and/or methylation status of genes that encode proteins involved in mismatch repair, including hPMS2, hMLH1, hMSH2, and hMSH6. A tumor having deficient expression of any one of these four is determined to have deficient mismatch repair (termed “dMMR”), while a tumor that is not deficient in expression of any of these genes is determined to have proficient MMR (termed “pMMR”). MMR status may be determined, for example, a protein-based assay (such as by immunoassay, such as a solid-phase enzyme immunoassay (e.g., ELISA) or affinity histochemical assay (AHC) assay) or a polymerase chain reaction (PCR) assay (such as a real-time reverse transcriptase PCR assay).

As used herein, a “human TAP biomarker-specific reagent” shall refer collectively to human TAP1 biomarker-specific reagents and human TAP2 biomarker-specific reagents.

As used herein, a “human TAP protein biomarker-specific reagent” shall refer collectively to human TAP1 protein biomarker-specific reagents and human TAP2 protein biomarker-specific reagents.

As used herein, a “TAP RNA biomarker-specific reagent” shall refer collectively to human TAPI RNA biomarker-specific reagents and human TAP2 RNA biomarker-specific reagents.

As used herein, an “anti-human TAP antibody” shall refer collectively to human anti-TAP1 antibodies and human anti-TAP2 antibodies.

As used herein, an “anti-human TAP monoclonal antibody” shall refer collectively to human anti-TAP1 monoclonal antibodies and human anti-TAP2 monoclonal antibodies.

As used herein, a “human TAP1 biomarker-specific reagent” shall refer collectively to human TAP1 protein biomarker-specific reagents and human TAP1 RNA biomarker-specific reagents.

As used herein, a “human TAP1 protein biomarker-specific reagent” shall refer to any biomarker specific reagent capable of specifically binding to SEQ ID NO: 1 (see Uniprot entry No. Q03518) in the context of a tumor sample derived from a human subject.

As used herein, a “human TAP1 RNA biomarker-specific reagent” shall refer to any biomarker specific reagent capable of specifically binding to an mRNA encoding SEQ ID NO: 1 or a cDNA thereof in the context of a tumor sample derived from a human subject, including but not limited to nucleic acid probes and primers complementary to such an mRNA or cDNA.

As used herein, an “anti-human TAP1 antibody” shall refer to any antibody or antibody fragment capable of specifically binding to SEQ ID NO: 1 in the context of a tumor sample derived from a human subject.

As used herein, an “anti-human TAP1 monoclonal antibody” shall refer to any monoclonal antibody (or fragment thereof) capable of specifically binding to SEQ ID NO: 1 in the context of a tumor sample derived from a human subject.

As used herein, a “human TAP2 biomarker-specific reagent” shall refer to any biomarker specific reagent capable of specifically binding to SEQ ID NO: 2 (or an mRNA encoding the same) (see Uniprot entry Q03519) in the context of a tumor sample derived from a human subject.

As used herein, a “human TAP2 protein biomarker-specific reagent” shall refer to any biomarker specific reagent capable of specifically binding to SEQ ID NO: 2 in the context of a tumor sample derived from a human subject.

As used herein, a “human TAP2 RNA biomarker-specific reagent” shall refer to any biomarker specific reagent capable of specifically binding to an mRNA encoding SEQ ID NO: 2 in the context of a tumor sample derived from a human subject.

As used herein, an “anti-human TAP2 antibody” shall refer to any antibody or antibody fragment capable of specifically binding to SEQ ID NO: 2 in the context of a tumor sample derived from a human subject.

As used herein, an “anti-human TAP2 monoclonal antibody” shall refer to any monoclonal antibody (or fragment thereof) capable of specifically binding to SEQ ID NO: 2 in the context of a tumor sample derived from a human subject.

As used herein, a “human pan-TAP biomarker-specific reagent” shall refer to a biomarker specific reagent capable of specifically binding to each of SEQ ID NO: 1 (or an mRNA encoding the same) and SEQ ID NO: 2 (or an mRNA encoding the same) in the context of a tumor sample derived from a human subject.

As used herein, a “human pan-TAP protein biomarker-specific reagent” shall refer to a biomarker specific reagent capable of specifically binding to each of SEQ ID NO: 1 and SEQ ID NO: 2 in the context of a tumor sample derived from a human subject.

As used herein, a “human pan-TAP RNA biomarker-specific reagent” shall refer to a biomarker specific reagent capable of specifically binding to each of an mRNA encoding SEQ ID NO: 1 and an mRNA encoding SEQ ID NO: 2 in the context of a tumor sample derived from a human subject.

As used herein, an “anti-human pan-TAP antibody” shall refer to an antibody capable of specifically binding to each of SEQ ID NO: 1 and SEQ ID NO: 2 in the context of a tumor sample derived from a human subject.

As used herein, an “anti-human pan-TAP monoclonal antibody” shall refer to a monoclonal antibody capable of specifically binding to each of SEQ ID NO: 1 and SEQ ID NO: 2 in the context of a tumor sample derived from a human subject.

As used herein, a “human pan-TAP biomarker-specific reagent cocktail” shall refer to a composition comprising each of a human TAPI biomarker-specific reagent and a human TAP2 biomarker-specific reagent.

As used herein, a “human pan-TAP protein biomarker-specific reagent cocktail” shall refer to a composition comprising each of a human TAPI protein biomarker specific reagent and a human TAP2 protein biomarker specific reagent.

As used herein, a “human pan-TAP RNA biomarker-specific reagent cocktail” shall refer to a composition comprising each of a human TAP1 RNA biomarker-specific reagent and a human TAP2 RNA biomarker-specific reagent.

As used herein, an “anti-human pan-TAP antibody cocktail” shall refer to a composition comprising each of an anti-human TAP1 antibody and an anti-human TAP2 antibody.

As used herein, an “anti-human pan-TAP monoclonal antibody cocktail” shall refer to a composition comprising each of an anti-human TAP1 monoclonal antibody and an anti-human TAP2 monoclonal antibody.

As used herein, a “human HLA biomarker-specific reagent” shall refer collectively to human HLA-A biomarker-specific reagents, human HLA-B biomarker-specific reagents, human HLA-C biomarker-specific reagents, and human pan-HLA biomarker-specific reagents.

As used herein, a “human HLA protein biomarker-specific reagent” shall refer collectively to human HLA-A protein biomarker-specific reagents, human HLA-B protein biomarker-specific reagents, human HLA-C protein biomarker-specific reagents, and human pan-HLA protein biomarker-specific reagents.

As used herein, a “human HLA RNA biomarker-specific reagent” shall refer collectively to human HLA-A mRNA biomarker-specific reagents, human HLA-B mRNA biomarker-specific reagents, human HLA-C mRNA biomarker-specific reagents, and human pan-HLA RNA biomarker-specific reagents.

As used herein, a “human anti-HLA antibody” shall refer collectively to human anti-HLA-A antibodies, human anti-HLA-B antibodies, human anti-HLA-C antibodies, and human pan-HLA antibodies.

As used herein, a “human anti-HLA monoclonal antibody” shall refer collectively to human anti-HLA-A monoclonal antibodies, human anti-HLA-B monoclonal antibodies, human anti-HLA-C monoclonal antibodies, and human pan-HLA monoclonal antibodies.

As used herein, a “human HLA-A biomarker-specific reagent” shall refer to any biomarker specific reagent capable of specifically binding to SEQ ID NO: 3 (or an mRNA encoding the same) (see Uniprot ID No. P04439-1), but not to SEQ ID NO: 4 (see Uniprot ID No. P01889-1) or SEQ ID NO: 5 (or an mRNA encoding the same) (see Uniprot ID No. P10321-1), in the context of a tumor sample derived from a human subject.

As used herein, a “human HLA-A protein biomarker-specific reagent” shall refer to any biomarker specific reagent capable of specifically binding to SEQ ID NO: 3, but not to SEQ ID NO: 4 or SEQ ID NO: 5, in the context of a tumor sample derived from a human subject.

As used herein, an “anti-human HLA-A antibody” shall refer to any antibody or antibody fragment capable of specifically binding to SEQ ID NO: 3, but not to SEQ ID NO: 4 or SEQ ID NO: 5, in the context of a tumor sample derived from a human subject.

As used herein, an “anti-human HLA-A monoclonal antibody” shall refer to any monoclonal antibody (or fragment thereof) capable of specifically binding to SEQ ID NO: 3, but not to SEQ ID NO: 4 or SEQ ID NO: 5, in the context of a tumor sample derived from a human subject.

As used herein, a “human HLA-B biomarker-specific reagent” shall refer to any biomarker specific reagent capable of specifically binding to SEQ ID NO: 4 (or an mRNA encoding the same), but not to SEQ ID NO: 3 or SEQ ID NO: 5, in the context of a tumor sample derived from a human subject.

As used herein, an “anti-human HLA-B antibody” shall refer to any antibody or antibody fragment capable of specifically binding to SEQ ID NO: 4, but not to SEQ ID NO: 3 or SEQ ID NO: 5, in the context of a tumor sample derived from a human subject.

As used herein, an “anti-human HLA-B monoclonal antibody” shall refer to any monoclonal antibody (or fragment thereof) capable of specifically binding to SEQ ID NO: 4, but not to SEQ ID NO: 3 or SEQ ID NO: 5, in the context of a tumor sample derived from a human subject.

As used herein, a “human HLA-C biomarker-specific reagent” shall refer to any biomarker specific reagent capable of specifically binding to SEQ ID NO: 5 (or an mRNA encoding the same), but not to SEQ ID NO: 3 or SEQ ID NO: 4 (or mRNA encoding the same), in the context of a tumor sample derived from a human subject.

As used herein, an “anti-human HLA-C antibody” shall refer to any antibody or antibody fragment capable of specifically binding to SEQ ID NO: 5, but not to SEQ ID NO: 3 or SEQ ID NO: 4, in the context of a tumor sample derived from a human subject.

As used herein, an “anti-human HLA-C monoclonal antibody” shall refer to any monoclonal antibody (or fragment thereof) capable of specifically binding to SEQ ID NO: 5, but not to SEQ ID NO: 3 or SEQ ID NO: 4, in the context of a tumor sample derived from a human subject.

As used herein, a “human pan-HLA biomarker-specific reagent” shall refer to any biomarker specific reagent capable of specifically binding to each of SEQ ID NO: 3-5 (or an mRNA encoding the same) in the context of a tumor sample derived from a human subject.

As used herein, an “anti-human pan-HLA antibody” shall refer to any antibody or antibody fragment capable of specifically binding to each of SEQ ID NO: 3-5 in the context of a tumor sample derived from a human subject.

As used herein, an “anti-human pan-HLA monoclonal antibody” shall refer to any monoclonal antibody (or fragment thereof) capable of specifically binding to each of SEQ ID NO: 3-5 in the context of a tumor sample derived from a human subject.

As used herein, a “human pan-HLA biomarker-specific reagent cocktail” shall refer to a composition comprising each of a human HLA-A biomarker-specific reagent, a human HLA-B biomarker-specific reagent, and a human HLA-C biomarker-specific reagent.

As used herein, an “anti-human pan-HLA antibody cocktail” shall refer to a composition comprising each of an anti-human HLA-A antibody, an anti-human HLA-B antibody, and an anti-human HLA-C antibody.

As used herein, an “anti-human pan-HLA monoclonal antibody cocktail” shall refer to a composition comprising each of an anti-human HLA-A monoclonal antibody, an anti-human HLA-B monoclonal antibody, and an anti-human HLA-C monoclonal antibody.

As used herein, a “human B2M biomarker-specific reagent” shall refer to any biomarker specific reagent capable of specifically binding to SEQ ID NO: 6 (or an mRNA encoding the same) (see Uniprot ID No. P61769-1) in the context of a tumor tissue sample derived from a human subject.

As used herein, an “anti-human B2M antibody” shall refer to any antibody or antibody fragment capable of specifically binding to SEQ ID NO: 6 in the context of a tumor tissue sample derived from a human subject.

As used herein, an “anti-human B2M monoclonal antibody” shall refer to any monoclonal antibody (or fragment thereof) capable of specifically binding to SEQ ID NO: 6 in the context of a tumor tissue sample derived from a human subject.

### Overview

Responsiveness to checkpoint-directed therapy has been shown to be predicted by tumor mutational screening, such as by evaluating tumor mutational burden, microsatellite instability, and mismatch repair. See Klempner and Sahin. The hypothesis is that an increase in tumor mutations correlates with increased presentation of neoantigens by the tumor, making the tumor appear more “foreign” to the immune system and therefore more likely to be susceptible to immune responses. Even among tumors known to harbor a large number of mutations, the response rates to immunotherapy are unfortunately often lower than would be expected. For example, in deficient mismatch repair (dMMR) endometrial cancer, which is highly immunogenic, response rates for the most common checkpoint inhibitor therapies are between 27% and 57%. See Green. Moreover, there are tumor types known to have a relatively low TMB that respond well to immunotherapy. For example, kidney cancer has among the lowest TMB of all cancers, but a high percentage of these patients respond to immunotherapy. See Yarchoan II.

Previous groups have presented two hypotheses for the source of neoantigens in kidney cancer. The first is that the mutations in kidney cancer are more “immunogenic” as compared with other cancer types. For example, there may be a higher frequency of frameshift mutations in kidney cancer as opposed to single nucleotide variants. The second hypothesis is that mutations in chromatin remodeling pathways, such as PBRM1, common in kidney cancer, lead to the reactivation of human endogenous retroviruses (hERVs), which triggers an increased level of tumor foreignness that is unrelated to TMB. However, a recent study found that different types of TMB and tumor-derived hERV expression do not predict response to immunotherapy, and the source of tumor foreignness in kidney cancer remains elusive. See Au. The authors, however, were able to identify expanded T cell clones in the tumor, indicative that the response to immunotherapy is due to a yet undiscovered source of neoantigen.

Recently, there have been reports of a unique state of cell foreignness induced by loss of the TAP complex: a heterodimer consisting of one TAP1 and one TAP2 polypeptide. The TAP complex loads processed peptide antigens onto MHC class I complexes for display to cytotoxic T cells. Loss of TAP expression therefore would be expected to reduce the presentation of neoantigens by the tumor. However, the loss of the TAP complex also unexpectedly leads to the display of neoantigens derived from non-mutated differentially processed self-proteins, termed “T cell epitopes associated with impaired peptide processing” or “TEIPP.” See Gigoux & Wolchok. It is believed that these self-peptides may appear more “foreign” than mutated neoantigens because the entire peptide is new and different, rather than a single amino acid. Many kidney cancer patients have loss of TAP expression with intact MHC-I. See Seliger.

Applicant has hypothesized that loss of expression of the TAP complex coupled with intact expression of the MHC class I complex would correlate with response to immunotherapies that rely on cytotoxic T-cell responses, such as checkpoint inhibitor therapy, T-cell bispecific therapies, cancer vaccines, and cell-based immunotherapies. To test this hypothesis, Applicant conducted a preliminary database analysis which revealed that low TAP complex expression is associated with improved survival in kidney cancer in general. Applicant further evaluated TAP and HLA-A (a constituent element of an MHC-I complex) expression in a set of 95 kidney cancer cases and observed that about 25% of tumors have a loss of TAP1 and/or TAP2 coupled with intact HLA-A. Interestingly, this is similar to the rate of response to immunotherapy in kidney cancer. See Yarchoan II. This data supports Applicant's hypothesis.

In view of the foregoing, the present disclosure provides methods comprising evaluating in cells, such as tumor cells, of a tumor sample the expression of constituent elements of the transporter associated with antigen processing complex (e.g., TAP1 and/or TAP2) and/or the expression of the constituent elements of the major histocompatibility complex class I (e.g., HLA and/or B2M), as well as manufactures, compositions of matter, and systems useful for performing such methods.

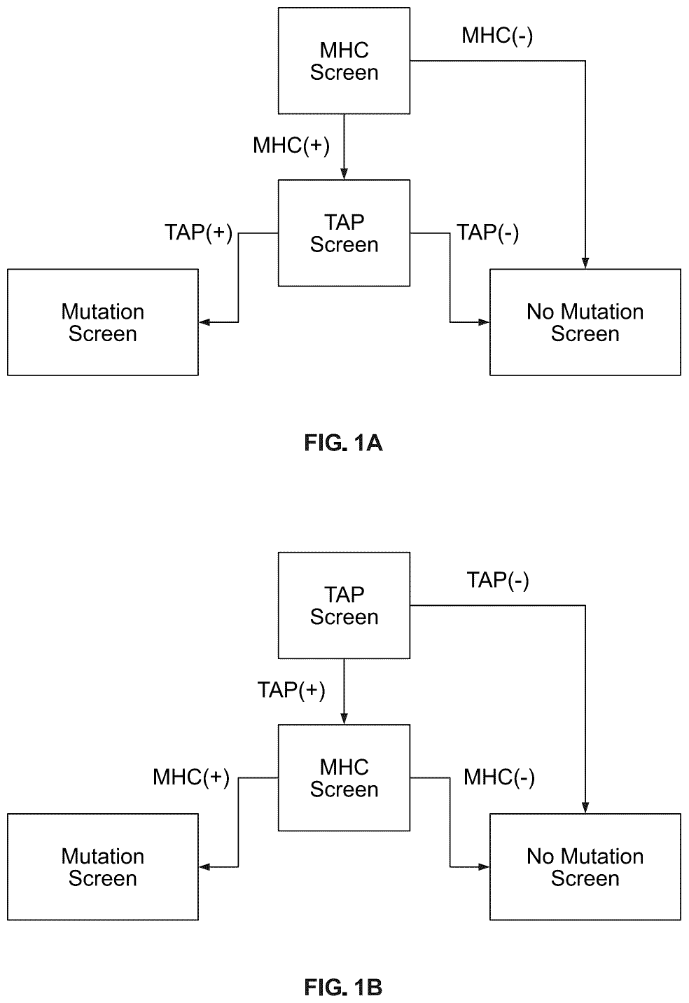

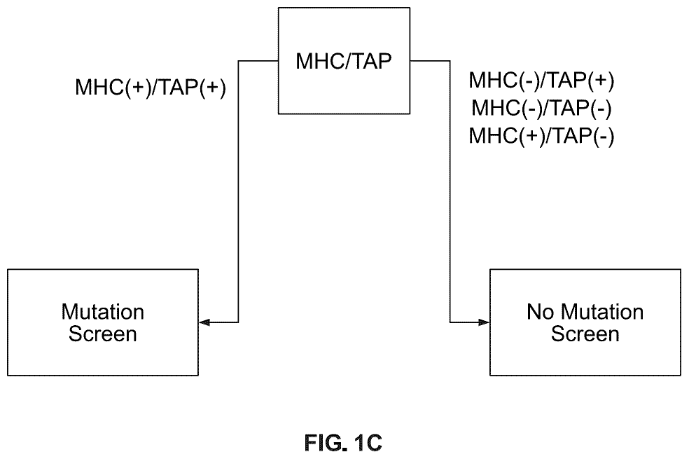

In some embodiments, the evaluation of the expression of the components of the APM may be used to determine whether the tumor should be referred for a tumor mutational screen, such as tumor mutational burden (TMB) screening, microsatellite instability (MSI) screening, and/or mismatch repair (MMR) screening. Tumors that are MHC(+)/TAP(+) are most likely to have their immunogenicity depend on the mutational load, and therefore are referred for tumor mutational screening. Tumors that are MHC(−) are unlikely to be able to present antigen and therefore are not likely to need a tumor mutational screen regardless of TAP status, unless otherwise indicated (for example, to determine a need for Lynch Syndrome screening). Tumors that are MHC(+)/TAP(−) are likely to be antigenic regardless of tumor mutational status and therefore are unlikely to benefit from additional tumor mutational screening unless otherwise indicated (for example, to determine a need for Lynch Syndrome screening). Exemplary workflows for screening and/or stratifying patients are presented in FIGS. 1A-1C.

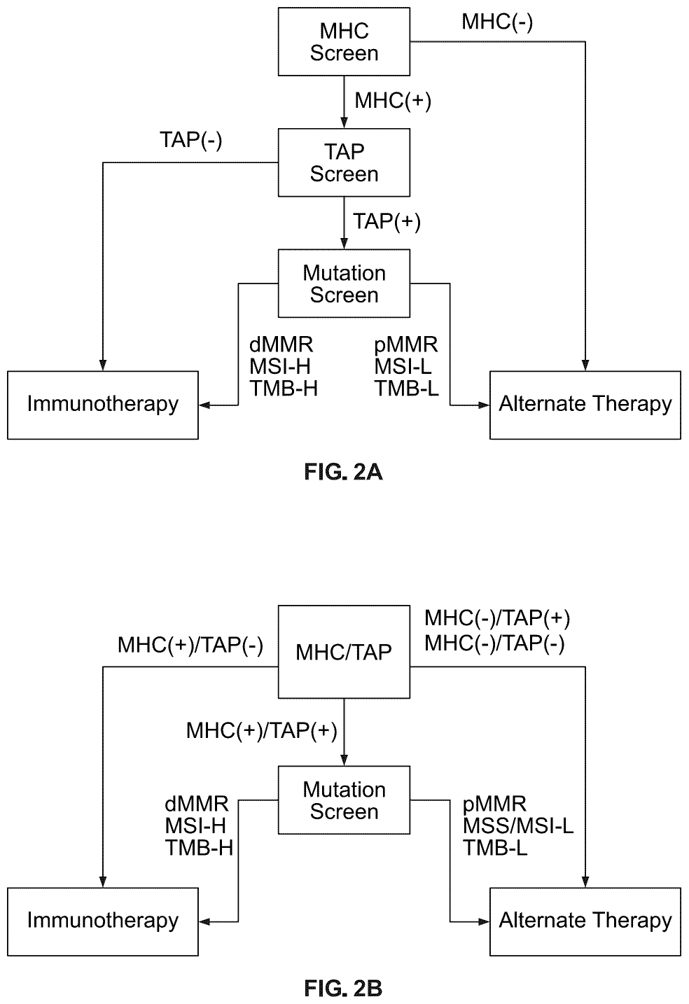

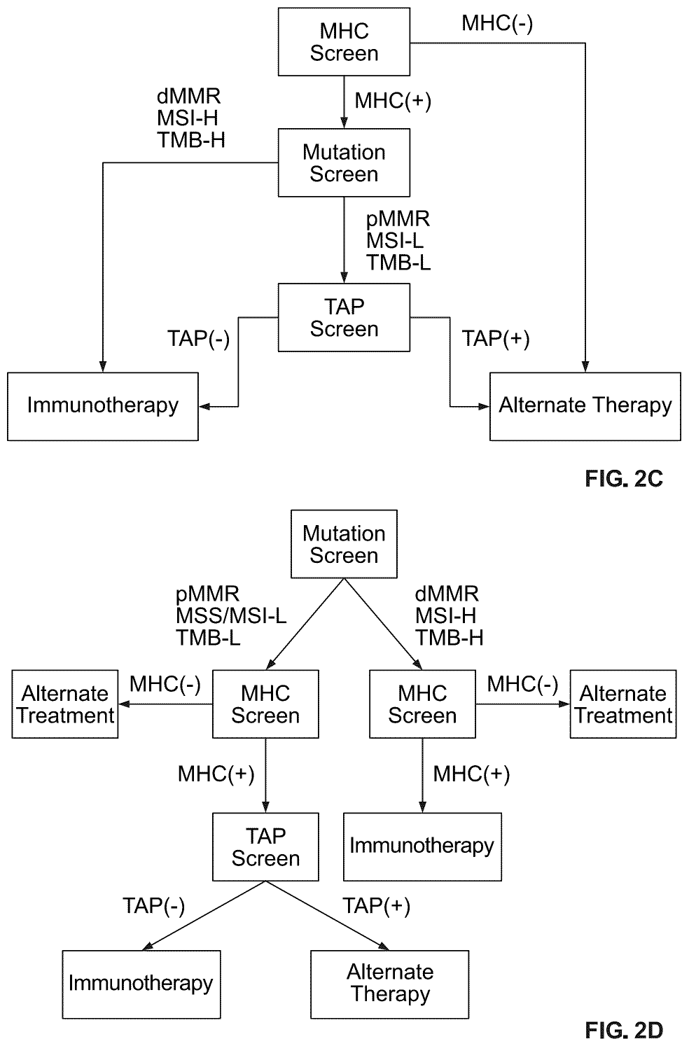

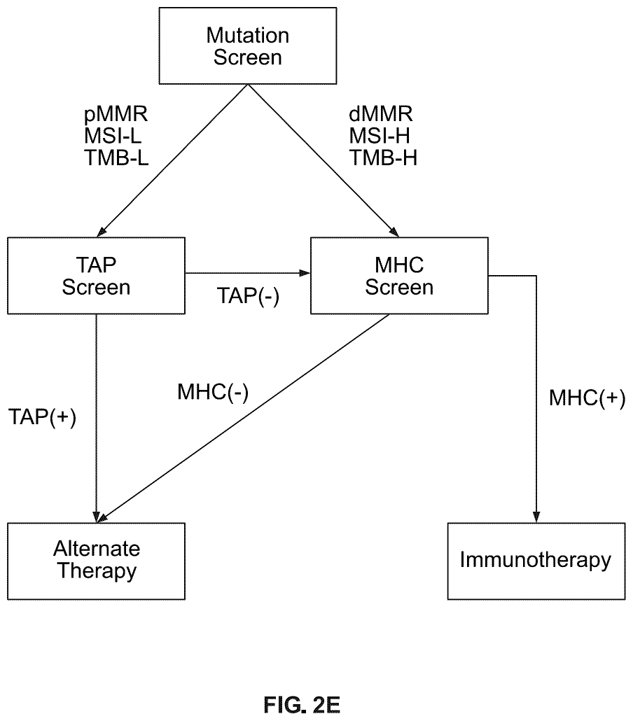

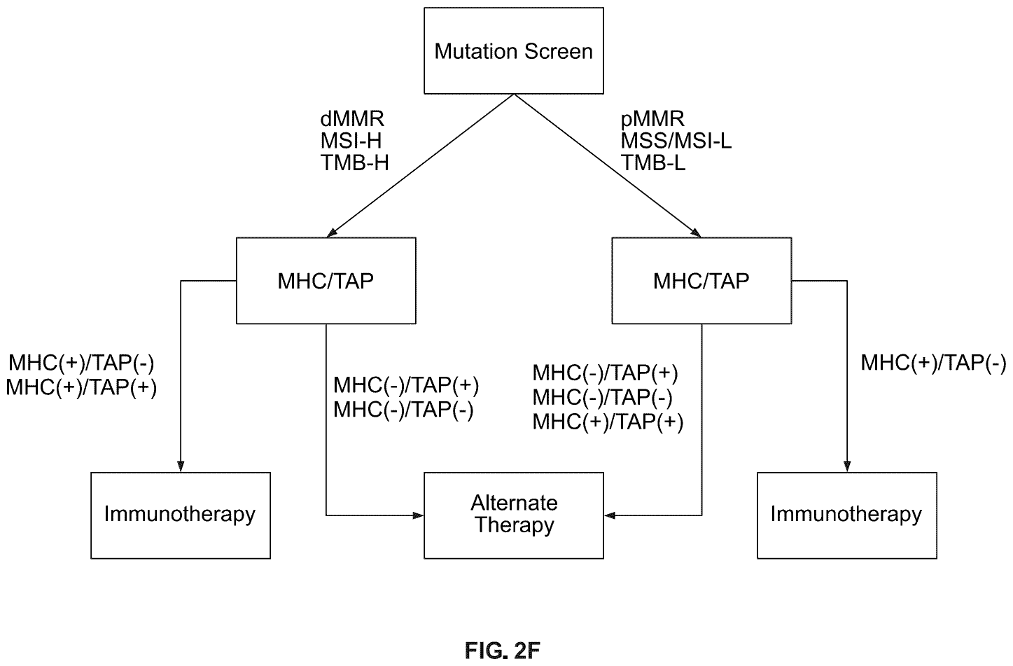

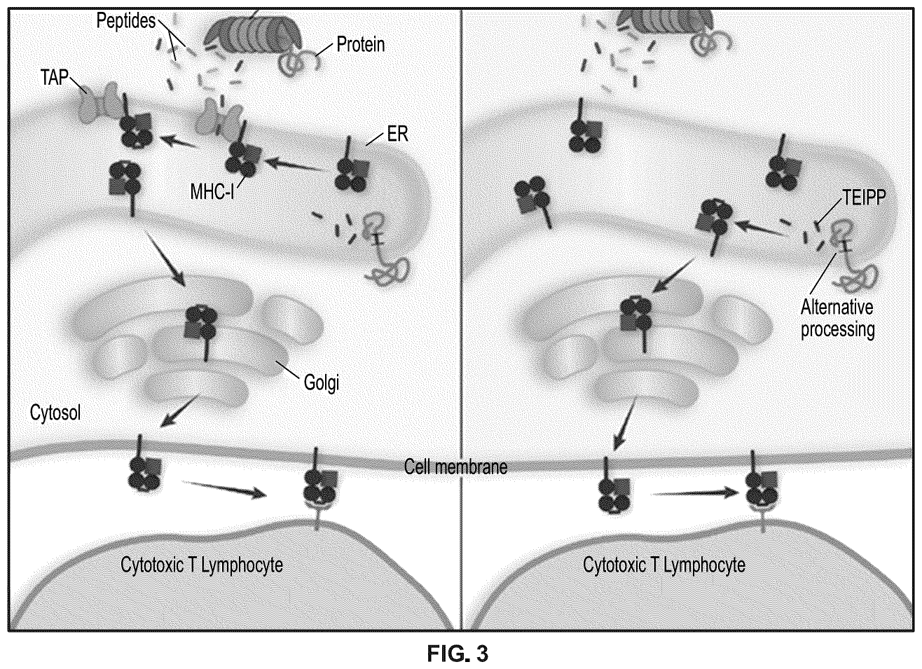

In some embodiments, the evaluation of the expression of the components of the APM may be used (optionally, in combination with tumor mutational screening) to determine whether the patient is likely to respond to MHC-I-dependent immunotherapeutic agents. In some embodiments, the subject is identified as a likely responder if a tumor sample from the subject is determined to have a loss of either or both of TAP1 and TAP2 and is determined to express a functional MHC class I complex (i.e., TAP(−)/MHC(+)). Subjects whose tumor sample is determined to have an intact TAP complex and is determined to express a functional MHC class I complex (i.e., TAP(+)/MHC(+)) may be subject to tumor mutational screening to determine likelihood of response to MHC-I-dependent immunotherapeutic agent and treated accordingly. Subjects whose tumor sample is determined to lack expression of at least one component of an MHC class I complex (MHC(−)) may be evaluated for an alternate therapy. See FIGS. 2A-2C.

In other embodiments, the evaluation of TAP complex and MHC-I complex may be used to further stratify patients whose tumors have been by previously evaluated by tumor mutational screening. For example, a tumor that previously has been determined to be pMMR, MSS/MSI-L, and/or TMB-L is assessed for the expression of TAP complex constituents and MHC-I complex constituents to determine which tumors may nonetheless be likely to respond to an MHC-I-dependent immunotherapeutic agent. Subjects in which the tumor sample is assessed to be TAP(−)/MHC(+) are identified as likely MHC-I-dependent immunotherapeutic agent responders and may be treated with an MHC-I-immunotherapeutic agent. Subjects in which the tumor sample is assessed to be TAP(+)/MHC(+), TAP(+)/MHC(−), or TAP(−)/MHC(−) are identified as unlikely MHC-I-dependent immunotherapeutic agent responders and may be treated with an alternate therapy. As another example, a tumor that previously has been determined to be dMMR, MSI-H, and/or TMB-H is assessed for the expression of MHC-I complex constituents (optionally along with TAP complex constituents) to determine which tumors may nonetheless be unlikely to respond to the MHC-I-dependent immunotherapeutic agent. Subjects in which the tumor sample is assessed to be MHC(+) are identified as likely MHC-I-dependent immunotherapeutic agent responders and may be treated with an MHC-I-dependent immunotherapeutic agent. Subjects in which the tumor sample is assessed to be MHC(−) are identified as unlikely MHC-I-dependent immunotherapeutic agent responders and may be treated with an alternate therapy (see FIGS. 2D-2F).

The TAP complex is a peptide loading complex and is composed of the TAP1 and TAP2 proteins. It is believed that a heterodimeric TAP complex is essential for peptide binding and translocation, i.e., both TAP1 and TAP2 must be present for peptide binding and translocation. On the other hand, it is believed that TAP1 or TAP2 homodimers are non-functional, i.e., TAP1 or TAP2 alone are non-functional. FIGS. 2A and 2B illustrate the correlation between TAP1 and TAP2 in kidney cancer (FIG. 2A) and in all cancers (FIG. 2B). Notably, FIGS. 2A and 2B depict that expression of both TAP1 and TAP2 are highly correlated in tumor samples.

Given that a functional TAP complex requires both the TAP1 and TAP2 proteins, and further given that the expression of both TAP1 and TAP2 is highly correlated in tumor samples, the expression of either TAPI alone or TAP2 alone serves as an adequate readout for the functionality of the TAP complex. In view of this, in some embodiments the loss of expression of either the TAP1 or TAP2 proteins correlates with a loss of functionality of the TAP complex. Said another way, if at least one of the TAP1 or TAP2 proteins are negatively expressed, then there is a loss of functionality of the TAP complex. On the other hand, intact or normal expression of TAP1 and TAP2 proteins correlates with intact functionality of the TAP complex. Again, since the expression levels of TAP1 and TAP2 highly correlate with one another, normal expression of TAP1 can be used as a proxy for the expression of TAP2 and vice-versa. In some embodiments, the expression of both the TAP1 and TAP2 proteins may be evaluated.

MHC-I is composed of a human leukocyte antigen (HLA) protein and a Beta-2-microglobulin (B2M) protein. Specifically, the HLA protein of MHC-I may be either HLA-A, HLA-B, or HLA-C. As used herein, the loss of expression of the HLA protein (either HLA-A, HLA-B, or HLA-C) or B2M correlates with a loss of functionality of the MHC-I. An intact or normal expression of the HLA protein or B2M, on the other hand, correlates with intact functionality of the MHC-I complex (also referred to herein as “MHC-I positive” or “MHC-I (+)”). As such, if HLA is positively expressed (HLA-A(+), HLA-B(+), or HLA-C(+)), then there is intact functionality of MHC-I.

Semi-quantitative and quantitative methods of assessing whether TAP1, TAP2, HLA, and/or optionally B2M are positive or are negative are described herein.

### Methods of Assessing the Expression of the Antigen Presenting Machinery

The present disclosure provides for methods of assessing the expression of the components of the APM and, in particular, the expression of the TAP complex and MHC-I complex.

In some embodiments, the assessment of the components of the APM, including the constituent elements of the TAP complex and the MHC-I complex, utilizes a detection method selected from flow cytometry, fluorescence-activated cell sorting (FACS) analysis, RNA sequencing (RNA-seq), polymerase chain reaction (PCR) (including quantitative PCR, real-time quantitative-PCR, multiplex quantitative-PCR, digital droplet PCR, etc.), spatial transcriptomics, spatial proteomics, mass spectrometry, RNA expression profiling (e.g. using molecular barcode chemistry (such as that provided by Nanostring)), or any combination thereof.

### A. Affinity Histochemical and Cytochemical Assays

In an exemplary embodiment, the expression of the TAP complex and MHC-I complex is assessed using affinity histochemical (AHC) or affinity cytochemical (ACC) techniques, such as immunohistochemistry (IHC), immunocytochemistry (ICC), and mRNA in situ hybridization (mRNA-ISH) assays. In the case of an AHC assay, the sample is a tissue section (including, but not limited to formalin-fixed paraffin embedded (FFPE) tissue sections and fresh frozen tissue sections). In the case of an ACC assay, the sample is a cytological sample (including, for example, fine needle aspirates and liquid-based cytology (LBC) samples). In the case of mRNA-ISH assay, the sample may be either a tissue section or a cytological sample. IHC assays are AHC assays in which the biomarker-specific reagent is an antibody. ICC assays are ACC assays in which the biomarker-specific reagent is an antibody.

AHC and ACC assays involve contacting a cellular sample with a biomarker-specific reagent under conditions that facilitate specific binding between the biomarker and the biomarker-specific reagent and unbound biomarker-specific reagent is removed from the sample (such as by washing with a wash buffer). If the biomarker-specific reagent is directly conjugated to a detectable moiety (termed a “direct detection AHC or ACC assay”), the sample may then be directly analyzed. Alternatively, the sample may be contacted with a set of detection reagents that interact with the biomarker-specific reagent to facilitate deposition a detectable moiety in close proximity the biomarker, thereby generating a detectable signal localized to the biomarker. Typically, wash steps are performed between application of different reagents to avoid non-specific staining of tissues. Biomarker-labeled samples may optionally be additionally labeled with a contrast agent (such as a hematoxylin stain) to visualize macromolecular structures within the cellular sample.

### A.1. Samples

Any type of cellular tumor sample compatible with AHC or ACC assays may be used.

In an exemplary embodiment, the cellular sample is a fixed cellular sample. Fixing a cellular sample preserves cells and tissue constituents in as close to a life-like state as possible and allows them to undergo preparative procedures without significant change. Autolysis and bacterial decomposition processes that begin upon cell death are arrested, and the cellular and tissue constituents of the sample are stabilized so that they withstand the subsequent stages of tissue processing. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum. The most commonly used fixative in preparing samples is formaldehyde, generally in the form of a formalin solution (formaldehyde in an aqueous (and typically buffered) solution). In an embodiment, the samples used in the present methods are fixed by a method comprising fixation in a formalin-based fixative. In one example, the fixative is 10% neutral buffered formalin. Notwithstanding these examples, the tissues can be fixed by process using any fixation medium that is compatible with the biomarker-specific reagents and specific detection reagents used.

In some embodiments, the fixed cellular sample is embedded in an embedding medium. An embedding medium is an inert material in which tissues and/or cells are embedded to help preserve them for future analysis. Embedding also enables cellular samples to be sliced into thin sections. Embedding media include paraffin, celloidin, OCT™ compound, agar, plastics, or acrylics. In an embodiment, the sample is fixed in a formalin-based fixative and embedded in paraffin to form a formalin-fixed, paraffin-embedded (FFPE) block.

In some embodiments if the cellular sample is embedded in paraffin, the sample can be deparaffinized using appropriate deparaffinizing process.

In some embodiments, the biological samples are pre-treated with an enzyme inactivation composition to substantially or completely inactivate endogenous peroxidase activity. For example, some cells or tissues contain endogenous peroxidase. Using an HRP conjugated antibody may result in high, non-specific background staining. This non-specific background can be reduced by pre-treatment of the sample with an enzyme inactivation composition as disclosed herein. In some embodiments, the samples are pre-treated with hydrogen peroxide only (about 1% to about 3% by weight of an appropriate pre-treatment solution) to reduce endogenous peroxidase activity. Once the endogenous peroxidase activity has been reduced or inactivated, detection kits may be added, followed by inactivation of the enzymes present in the detection kits, as provided above. The disclosed enzyme inactivation composition and methods can also be used as a method to inactivate endogenous enzyme peroxidase activity. Additional inactivation compositions are described in U.S. Publication No. 2018/0120202, the disclosure of which is hereby incorporated by reference herein in its entirety.

### A.2. Automated Staining Systems

The AHC/ACC assays described herein may be performed on an automated staining apparatus, manually, or feature a combination of automated steps and manual steps. In some embodiments, an automated staining apparatus includes one or more reservoirs (such as for storage of the various reagents used in the labeling protocols), one or more reagent dispense units in fluid communication with the one or more reservoirs for dispensing reagent to onto a sample, a waste removal system for removing used reagents and other waste from the sample, and a control system that coordinates the actions of the one or more reagent dispense units and a waste removal system. In addition to performing labeling steps, an automated staining apparatus may be configured to perform steps ancillary to labeling (or are compatible with separate systems that perform such ancillary steps), including, but not limited to, slide baking (for adhering the sample to a slide), dewaxing (also referred to as deparaffinization), antigen retrieval, counterstaining, dehydration and clearing, and coverslipping.

Prichard, Overview of Automated Immunohistochemistry, Arch Pathol Lab Med., Vol. 138, pp. 1578-1582 (2014), the disclosure of which is hereby incorporated herein by reference in its entirety, describes several specific examples of automated staining apparatus and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and LAB VISION AUTOSTAINER (Thermo Scientific) automated AHC labeling systems. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published patents application Nos. 20030211630 and 20040052685, each of which is incorporated herein by reference in its entirety.

An automated staining apparatus typically operates on one of the following principles: (1) open individual slide labeling, in which slides are positioned horizontally and reagents are dispensed as a puddle on the surface of the slide containing a tissue sample (such as implemented on the DAKO AUTOSTAINER Link 48 (Agilent Technologies) and INTELLIPATH (Biocare Medical) labelers); (2) liquid overlay technology, in which reagents are either covered with or dispensed through an inert fluid layer deposited over the sample (such as implemented on BENCHMARK and DISCOVERY labelers); (3) capillary gap labeling, in which the slide surface is placed in proximity to another surface (which may be another slide or a coverplate) to create a narrow gap, through which capillary forces draw up and keep liquid reagents in contact with the samples (such as the labeling principles used by DAKO TECHMATE, Leica BOND, and DAKO OMNIS labelers).

Some iterations of capillary gap labeling do not mix the fluids in the gap (such as on the DAKO TECHMATE and the Leica BOND). In variations of capillary gap labeling termed dynamic gap labeling, capillary forces are used to apply sample to the slide, and then the parallel surfaces are translated relative to one another to agitate the reagents during incubation to effect reagent mixing (such as the labeling principles implemented on DAKO OMNIS slide labelers (Agilent)). In translating gap labeling, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap (see WO 2011/139978A1, the disclosure of which is hereby incorporated by reference herein in its entirety). It has also been proposed to use inkjet technology to deposit reagents on slides (see WO 2016/170008A1, the disclosure of which is hereby incorporated by reference herein in its entirety). This list of labeling technologies is not intended to be comprehensive, and any fully or semi-automated system or manual method for performing biomarker labeling may be incorporated into the present methods.

### A.4. Detection Reagents and Detectable Moieties

In some embodiments, the specific binding agent (e.g., a monoclonal antibody) includes or is conjugated to a detectable moiety (e.g., a fluorescent molecule or a mass spectrometer-detectable label). In other embodiments, the biomarker-specific reagent does not include a detectable moiety. In these embodiments, the sample is then contacted with a set of detection reagents that interact with the specific binding agent to facilitate deposition of a detectable moiety in close proximity the biomarker, thereby generating a detectable signal localized to the biomarker. Typically, wash steps are performed between application of different reagents to prevent unwanted non-specific labeling of tissues.

Any detection reagents or detectable moieties compatible with simplex or multiplex immunohistochemistry or immunocytochemistry may be utilized in the methods of the present disclosure. In some embodiments, the detectable moiety is a fluorophore. Non-limiting examples fluorophores include coumarins, fluoresceins (or fluorescein derivatives and analogs), rhodamines, resorufins, luminophores and cyanines. Other examples of fluorophores include, but are not limited to, 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl) maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); 2′,7′-difluorofluorescein (OREGON GREEN™); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine×isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Yet other examples of fluorescent molecules can be found in Molecular Probes Handbook—A Guide to Fluorescent Probes and Labeling Technologies, Molecular Probes, Eugene, OR, ThermoFisher Scientific, 11^{th} Edition', the disclosure of which is incorporated by reference herein in its entirety.

In other embodiments, the detectable moiety is a molecule detectable via brightfield microscopy. Non-limiting examples of brightfield dyes compatible with IHC, including multiplex IHC, and methodologies of using the same are disclosed in U.S. Pat. No. 10,041,950, the disclosure of which is hereby incorporated by reference herein in its entirety. Specific examples include diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), and Rhodamine 110 (Rhodamine), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, or tetrazolium violet.

Non-limiting examples of suitable detectable conjugates including different detectable moieties are disclosed in PCT Publication No. WO/2022/043491, the disclosure of which is hereby incorporated by reference in its entirety. For instance, PCT Publication No. WO/2022/043491 discloses detectable moieties having different “core” structures, e.g., a coumarin core, a phenoxazinone core, a 4-Hydroxy-3-phenoxazinone core, a 7-amino-4-Hydroxy-3-phenoxazinone core, a thioninium core, a phenoxazine core, a phenoxathiin-3-one core, or a xanthene core. Any of these detectable moieties may be suitable for labeling the constituent elements of the TAP complex or the MHC-I complex.

In yet other embodiments, the detectable moiety is a mass spectrometer-detectable label, including any of those disclosed in U.S. Pat. Nos. 10,883,999, 10,078,083, and 9,291,597, the disclosures of which are hereby incorporated by reference herein in their entireties.

Other detection reagents, detectable moieties, and detection strategies are described in U.S. Pat. Nos. 11,249,085, 11,249,085, and 10,168,336; and in United States Patent Application Publication No. 2012/0171668, the disclosures of which are hereby incorporated by reference herein in their entireties.

Non-limiting examples of commercially available detection reagents or kits comprising detection reagents suitable for use with present methods include: VENTANA ULTRAVIEW detection systems (secondary antibodies conjugated to enzymes, including HRP and AP); VENTANA IVIEW detection systems (biotinylated anti-species secondary antibodies and streptavidin-conjugated enzymes); VENTANA OPTIVIEW detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OPTIVIEW Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OMNIMAP, DISCOVERY ULTRAMAP anti-hapten antibody, secondary antibody, chromogen, fluorophore, and dye kits, each of which are available from Ventana Medical Systems, Inc. (Tucson, Arizona); POWERVISION and POWERVISION+IHC Detection Systems (secondary antibodies directly polymerized with HRP or AP into compact polymers bearing a high ratio of enzymes to antibodies); DAKO ENVISION™+ System (enzyme labeled polymer that is conjugated to secondary antibodies); ULTRAPLEX Multiplex Chromogenic IHC Technology from CELL IDx (hapten-labeled primary antibodies combined with enzyme-labeled or fluor-labeled anti-hapten secondary antibodies).

### A.5. Counterstaining and Morphological Staining

In some embodiments, the cellular tumor samples may be counterstained to assist in identifying morphologically relevant areas, either manually or automatically. Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst S769121, stains yellow under neutral pH and stains blue under acidic pH), DRAQ5 (stains red), DRAQ7 (stains red); fluorescent non-nuclear stains, such as fluorophore-labelled phalloidin, (stains filamentous actin, color depends on conjugated fluorophore).

Where the method is an AHC method, it may also be desirable to morphologically stain a serial section of the biomarker-labeled section, which can be used to identify particular regions of interest in which to evaluate the biomarker-stained sample. Many morphological stains are known, including but not limited to, hematoxylin and eosin (H&E) stain and Lee's Stain (Methylene Blue and Basic Fuchsin). In a specific embodiment, at least one serial section of each biomarker-labeled slide is H&E stained. Any method of applying H&E stain may be used, including manual and automated methods. In an embodiment, at least one section of the sample is an H&E stained sample stained on an automated staining system. Automated systems for performing H&E staining typically operate on one of two staining principles: batch staining (also referred to as “dip 'n dunk”) or individual slide staining. Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA HE 600 series H&E stainers (individual slide stainer) from Roche; the DAKO COVERSTAINER from Agilent Technologies (batch stainer); the LEICA ST4020 Small Linear Stainer, LEICA ST5020 MULTISTAINER, and the LEICA ST5010 AUTOSTAINER XL series H&E stainers from Leica Biosystems Nussloch GmbH (batch stainers).

### A.6. Simplex AHC/ACC Assays

In an embodiment, the AHC or ACC assay is provided in a simplex format. In a simplex format, a single detectable moiety is used for all biomarker-specific reagents bound to the sample. Thus, for example, an IHC assay for a single biomarker using a single chromogen or fluorophore would be considered a “simplex IHC assay.” Likewise, and IHC assay that stains 3 different biomarkers on the same sample using the same chromogen or fluorophore would also be considered a “simplex IHC assay.” Where multiple biomarker-specific agents are used in a simplex format, they can be applied to the sample separately or via a biomarker-specific reagent cocktail. In either case, the set of detection reagents should be selected to effect deposition of the same detectable moiety in proximity to each of the biomarker-specific reagents.

In some embodiments, the detectable moiety used in the simplex assay is a fluorophore. Exemplary fluorophores include several common chemical classes, such as coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11th Edition. Exemplary fluorescent dyes compatible with mpIHC/mpICC and methodologies of using the same are disclosed at, for example, Gorris, Hofman, and Parra.

In other embodiments, the detectable moiety used in the simplex assay is a molecule detectable via brightfield microscopy. Exemplary brightfield dyes compatible with multiplex IHC and methodologies of using the same are disclosed at, for example, Hofman, Ide, Morrison, Parra, Stack, and U.S. Pat. No. 10,041,950 B2. Specific examples include diaminobenzidine (DAB), 4-(dimethylamino) azobenzene-4′-sulfonamide (DABSYL), tetramethylrhodamine (TAMRA), N,N′-biscarboxypentyl-5,5′-disulfonato-indo-dicarbocyanine (Cy5), and Rhodamine 110 (Rhodamine).

In yet other embodiments, the detectable moiety used in the simplex assay is a mass spectrometer-detectable label. Reviews of mass spectrometry-based multiplexing methods and labels can be found at Levenson and Parra, for example.

In yet other embodiments, the detectable moiety used in the simplex assay is a nucleic acid barcode. As used in this context, a nucleic acid barcode is an oligonucleotide molecule conjugated to a biomarker-specific reagent in a manner that the oligonucleotide can be localized to a particular location in the sample. Exemplary nucleic acid barcode detection chemistry includes those used by PHENOCYCLER Technology from Akoya Biosystems, Inc. (specific oligonucleotides conjugated to biomarker-specific reagents or secondary detection reagents and complementary to fluorophore-conjugated reporter oligonucleotides), Digital Spatial Profiling (DSP) technology provided by Nanostring, Inc. (oligonucleotides conjugated to biomarker-specific reagents or secondary detection reagents via a photocleavable linker; upon cleavage of the linker, the oligonucleotide is identified, quantified, and mapped back to the tissue location), and INSITUPLEX technology from Ultivue, Inc. (specific oligonucleotides conjugated to biomarker-specific reagents or secondary detection reagents, which can be amplified to increase the ratio of barcodes per antibody and then bound to a complementary labeled reporter probe). See Tan (reviewing several multiplex methodologies).

A.6.a. Simplex AHC/ACC Methods Using Human HLA-A, HLA-B, and/or HLA-C Biomarker-Specific Reagents

A simplex AHC or ACC staining method is provided, said method comprising: (a) contacting a cellular tumor sample with one or more a human HLA biomarker-specific reagent(s) (such as a human HLA-A biomarker-specific reagent, a human HLA-B biomarker-specific reagent, a human HLA-C biomarker-specific reagent, a human pan-HLA biomarker-specific reagent, a human pan-HLA biomarker-specific reagent, or a human pan-HLA biomarker-specific reagent cocktail) under conditions that permit specific binding of the biomarker-specific reagent to the cellular tumor sample; and (b) removing unbound biomarker-specific reagent from the sample, thereby obtaining a labeled cellular tumor sample. In some embodiments, the biomarker-specific reagent(s) is/are directly conjugated to a detectable moiety, in which case the sample is ready for evaluation. In other embodiments, the method further comprises (c) contacting the labeled cellular tumor sample with a set of detection reagents that interact with the one or more biomarker-specific reagents to facilitate deposition of a detectable moiety on the labeled cellular tumor sample.

In an embodiment, the human HLA biomarker-specific reagent is a human HLA-A biomarker-specific reagent, a human HLA-B, or a human HLA-C biomarker-specific reagent. In such an embodiment, the cellular tumor sample is stained for only one of the MHC-I-associated HLA gene products. It is believed that such an embodiment has an advantage of potentially being more cost-effective and easier to develop than alternative methods of identifying MHC status in any of the workflows illustrated in FIGS. 1A-2F.

As yet another example, a first cellular sample from the tumor could be stained for HLA-A, a second cellular sample of the tumor could be stained for HLA-B, and a third cellular sample of the tumor could be stained for HLA-C. In such an embodiment, the HLA expression status for the workflows illustrated in FIGS. 1A-2F may be based on the expression level of any or all of HLA-A, HLA-B and HLA-C.

As yet another example, a first cellular sample from the tumor could be stained for HLA-A, a second cellular sample of the tumor could be stained for HLA-B if the first cellular sample is HLA-A(−), and a third cellular sample of the tumor could be stained for HLA-C if the second cellular sample is HLA-B(−). In such an embodiment, the HLA expression status for the workflows illustrated in FIGS. 1A-2F may be based on the expression level of any or all of HLA-A, HLA-B and HLA-C.

In another embodiment, the one or more human HLA biomarker-specific reagents are each of a human HLA-A biomarker-specific reagent, a human HLA-B biomarker-specific reagent, and a human HLA-C biomarker-specific reagent (including use of a human pan-HLA biomarker specific reagent cocktail and separate addition of each of HLA-A, HLA-B, and HLA-C biomarker-specific reagents). In another embodiment, the HLA biomarker-specific reagent is a human pan-HLA biomarker-specific reagent. In such embodiments, the cellular tumor sample is stained for each of human HLA-A, HLA-B, and HLA-C. One advantage of such a method is that it includes analysis of all 3 biomarkers in a simplex format, which minimizes the amount of sample required to use the assay. Such simplex methods could be useful in, for example, the workflows illustrated in FIGS. 1A-2F.

In some embodiments of any of the foregoing simplex AHC and simplex ACC assays for HLA-A, HLA-B, and/or HLA-C, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen. Additionally, or alternatively, the cellular tumor sample is from a tumor previously determined to express of one or more of human TAP1 and/or human TAP2. In a specific embodiment, the tumor is human TAP1(+). In another specific embodiment, the tumor is human TAP2(+). In another specific embodiment, the tumor is human TAP1(−). In another specific embodiment, the tumor is human TAP2(−). In another specific embodiment, the tumor is human pan-TAP(+). In another specific embodiment, the tumor is human pan-TAP(−).

In a specific embodiment, the tumor is dMMR/human TAP1(+). In another specific embodiment, the tumor is dMMR/human TAP2(+). In another specific embodiment, the tumor is dMMR/human pan-TAP(+). In a specific embodiment, the tumor is dMMR/human TAP1(−). In another specific embodiment, the tumor is dMMR/human TAP2(−). In another specific embodiment, the tumor is dMMR/human pan-TAP(−). In a specific embodiment, the tumor is pMMR/human TAP1(+). In another specific embodiment, the tumor is pMMR/human TAP2(+). In another specific embodiment, the tumor is pMMR/human pan-TAP(+). In a specific embodiment, the tumor is pMMR/human TAP1(−). In another specific embodiment, the tumor is pMMR/human TAP2(−). In another specific embodiment, the tumor is pMMR/human pan-TAP(−).

In a specific embodiment, the tumor is MSI-H/human TAP1(+). In another specific embodiment, the tumor is MSI-H/human TAP2(+). In another specific embodiment, the tumor is MSI-H/human pan-TAP(+). In a specific embodiment, the tumor is MSI-H/human TAP1(−). In another specific embodiment, the tumor is MSI-H/human TAP2(−). In another specific embodiment, the tumor is MSI-H/human pan-TAP(−). In a specific embodiment, the tumor is MSI-L/human TAP1(+). In another specific embodiment, the tumor is MSI-L/human TAP2(+). In another specific embodiment, the tumor is MSI-L/human pan-TAP(+). In a specific embodiment, the tumor is MSI-L/human TAP1(−). In another specific embodiment, the tumor is MSI-L/human TAP2(−). In another specific embodiment, the tumor is MSI-L/human pan-TAP(−).

In a specific embodiment, the tumor is TMB-H/human TAP1(+). In another specific embodiment, the tumor is TMB-H/human TAP2(+). In another specific embodiment, the tumor is TMB-H/human pan-TAP(+). In a specific embodiment, the tumor is TMB-H/human TAP1(−). In another specific embodiment, the tumor is TMB-H/human TAP2(−). In another specific embodiment, the tumor is TMB-H/human pan-TAP(−). In a specific embodiment, the tumor is TMB-L/human TAP1(+). In another specific embodiment, the tumor is TMB-L/human TAP2(+). In another specific embodiment, the tumor is TMB-L/human pan-TAP(+). In a specific embodiment, the tumor is TMB-L/human TAP1(−). In another specific embodiment, the tumor is TMB-L/human TAP2(−). In another specific embodiment, the tumor is TMB-L/human pan-TAP(−).

Exemplary antibodies useful for the present methods are set forth in Table 1:

TABLE 1
Table of anti-human HLA antibodies.

| Vendor | Clone ID | Clonality | Specificity |
|---|---|---|---|
| Abcam | EP1395Y | Monoclonal | HLA-A |
| | Ab193415 | Polyclonal | HLA-B |
| | EPR6749 | Monoclonal | HLA-C |
| | Ab193432 | Polyclonal | HLA-C |
| | (Catalog | | |
| | Number) | | |
| | EMR8-5 | Monoclonal | Pan-HLA |
| | EPR22172 | Monoclonal | Pan-HLA |
| | MEM-123 | Monoclonal | Pan-HLA |
| | MEM-147 | Monoclonal | Pan-HLA |
| | YTH862.2 | Monoclonal | Pan-HLA |
| | EPR1394Y | Monoclonal | HLA-A, HLA-B, and |
| | | | maybe HLA-C |
| Thermo Fisher | 2G5 | Monoclonal | HLA-A |
| Scientific | W6/32 | Monoclonal | Pan-HLA |
| | HC10 | Monoclonal | Reacts mostly with HLA- |
| | | | B and HLA-C, and with |
| | | | some HLA-A clones |
| Fisher Scientific | MEM-81 | Monoclonal | HLA-A |
| Beckman Coulter | B9.12.1 | Monoclonal | Pan-HLA |
| Santa Cruz | LY5.1 | Monoclonal | Pan-HLA |
| Biotechnology | HP-1F7 | Monoclonal | Pan-HLA |
| GeneTex | YTH 862.2 | Monoclonal | Pan-HLA |
| Leinco | BB7.6 | Monoclonal | HLA-B |
| Technologies | | | |
| Miltenyi Biotec | REA145 | Monoclonal | HLA-B8 |

A.6.b. Simplex AHC/ACC Methods Using Human TAP Biomarker-Specific Reagents

A simplex AHC or ACC staining method is provided, the method comprising: (a) contacting a cellular tumor sample with one or more a human TAP biomarker-specific reagent(s) (such as a human TAPI biomarker-specific reagent, a human TAP2 biomarker-specific reagent, a human TAP1 biomarker-specific reagent and a human TAP2 biomarker-specific reagent, or a human pan-TAP biomarker-specific reagent) under conditions that permit specific binding of the biomarker-specific reagent to the cellular tumor sample; and (b) removing unbound biomarker-specific reagent from the sample, thereby obtaining a labeled cellular tumor sample. In some embodiments, the biomarker-specific reagent(s) is/are directly conjugated to a detectable moiety, in which case the sample is ready for evaluation. In other embodiments, the method further comprises (c) contacting the labeled cellular tumor sample with a set of detection reagents that interact with the one or more biomarker-specific reagents to facilitate deposition of a detectable moiety on the labeled cellular tumor sample.

In some embodiments, the human TAP biomarker-specific reagent is a human TAP1 biomarker-specific reagent or a human TAP2 biomarker-specific reagent. In such an embodiment, the cellular tumor sample is stained for human TAP1 or human TAP2, but not for both human TAP1 and human TAP2. There could be several utilities for such a method. For example, TAP1 and TAP2 expression are highly correlated, meaning when one is present the other is highly likely to be present as well, while the loss of expression of one means the other is highly likely to be lost as well. As such, a stain for only one could be used as a proxy for the status of both in any of the workflows illustrated in in FIG. 1A-FIG. 2E. As yet another example, a first cellular sample from the tumor could be stained for TAP1 and a second cellular sample of the tumor could be stained for TAP2. In such an embodiment, the TAP expression status for the workflows illustrated in FIG. 1A-FIG. 2E may be based on the expression level of both TAP1 and TAP2.

In another embodiment, the one or more human TAP biomarker-specific reagents are a human TAP1 biomarker-specific reagent and a human TAP2 biomarker-specific reagent. In such an embodiment, the cellular tumor sample is stained for both human TAP1 and human TAP2. One advantage of such a method is that it includes analysis of both markers in a simplex format, which increases the confidence that all loss of TAP is being detected while minimizing the amount of sample left over. Since such methods stain both TAP1 and TAP2, the sensitivity should be optimized in order to avoid expression of one of the TAPs at low levels to appear as though TAP is intact. An exemplary method for doing so is to titer the biomarker-specific reagents or adjust the staining conditions such that the intensity of specific staining of both markers is easily distinguishable from the intensity of specific staining of only one of the markers. As yet another example, a quantitative histochemical or cytochemical method such as quantitative IHC or quantitative RNA in situ hybridization methods. See Jenson (describing an exemplary quantitative IHC method) and Jamalzadeh (describing an exemplary quantitative RNA-ISH method). Such simplex methods could be useful in, for example, the workflows illustrated in FIG. 1A-FIG. 2E.

In some embodiments of any of the foregoing simplex AHC and simplex ACC assays for TAP1 and/or TAP2, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen. Additionally, or alternatively, the cellular tumor sample is from a tumor previously determined to express of one or more of human HLA-A, human HLA-B, and/or human HLA-C. In a specific embodiment, the tumor is HLA-A(+). In another specific embodiment, the tumor is HLA-B(+). In another specific embodiment, the tumor is HLA-C(+). In another specific embodiment, the tumor is pan-HLA(+). In a specific embodiment, the tumor is dMMR/HLA-A(+). In another specific embodiment, the tumor is dMMR/HLA-B(+). In another specific embodiment, the tumor is dMMR/HLA-C(+). In another specific embodiment, the tumor is dMMR/pan-HLA(+). In a specific embodiment, the tumor is pMMR/HLA-A(+). In another specific embodiment, the tumor is pMMR/HLA-B(+). In another specific embodiment, the tumor is pMMR/HLA-C(+). In another specific embodiment, the tumor is pMMR/pan-HLA(+). In a specific embodiment, the tumor is MSI-H/HLA-A(+). In another specific embodiment, the tumor is MSI-H/HLA-B(+). In another specific embodiment, the tumor is MSI-H/HLA-C(+). In another specific embodiment, the tumor is MSI-H/pan-HLA(+). In a specific embodiment, the tumor is MSI-L/HLA-A(+). In another specific embodiment, the tumor is MSI-L/HLA-B(+). In another specific embodiment, the tumor is MSI-L/HLA-C(+). In another specific embodiment, the tumor is MSI-L/pan-HLA(+). In a specific embodiment, the tumor is TMB-H/HLA-A(+). In another specific embodiment, the tumor is TMB-H/HLA-B(+). In another specific embodiment, the tumor is TMB-H/HLA-C(+). In another specific embodiment, the tumor is TMB-H/pan-HLA(+). In a specific embodiment, the tumor is TMB-L/HLA-A(+). In another specific embodiment, the tumor is TMB-L/HLA-B(+). In another specific embodiment, the tumor is TMB-L/HLA-C(+). In another specific embodiment, the tumor is TMB-L/pan-HLA(+).

Exemplary TAP1 and TAP2 antibodies that could be used in the present methods are set forth in Table 2 and Table 3:

TABLE 2
TAP1 antibodies

| Vendor | Clone ID | Clonality |
|---|---|---|
| Creative Diagnostics | 4E5 | Monoclonal |
| | CPBT-67438RH | Polyclonal |
| | (Catalog Number) | |
| Thermo Fisher Scientific | 53H8 | Monoclonal |
| | JE40-63 | Monoclonal |
| | 3D4 | Monoclonal |
| VWR | 1B11 | Monoclonal |
| | 2B4 | Monoclonal |
| | 3D10 | Monoclonal |
| Novus Biologicals | TAP1.28 | Monoclonal |
| Developmental Studies | CPTC-TAP1-1 | Monoclonal |
| Hybridoma Bank (DSHB) | | |
| Cell Signaling Technologies | E4T4F | Monoclonal |

TABLE 3
TAP2 antibodies

| Vendor | Clone ID | Clonality |
|---|---|---|
| Novus Biologicals | TAP2.17 | Monoclonal |
| DSHB | CPTC-TAP2-1 | Monoclonal |
| Abcam | Ab180611 (Catalog | Polyclonal |
| | Number) | |

Another example of an anti-human TAPI antibody is clone S14H22L21, available from Ventana Medical Systems, Inc. The preparation and characterization of the clone S14H22L21 is described herein in Examples 1 and 2.

A.6.c. Simplex AHC/ACC Methods Using Human B2M Biomarker-Specific Reagents

A simplex AHC or ACC staining method is provided, the method comprising: (a) contacting a cellular tumor sample with one or more a human B2M biomarker-specific reagent(s) under conditions that permit specific binding of the biomarker-specific reagent to the cellular tumor sample; and (b) removing unbound biomarker-specific reagent from the sample, thereby obtaining a labeled cellular tumor sample. In some embodiments, the biomarker-specific reagent(s) is/are directly conjugated to a detectable moiety, in which case the sample is ready for evaluation. In other embodiments, the method further comprises (c) contacting the labeled cellular tumor sample with a set of detection reagents that interact with the one or more biomarker-specific reagents to facilitate deposition of a detectable moiety on the labeled cellular tumor sample. Such an embodiment may be useful as a proxy for intact MHC class I machinery expression, for example, in the workflows illustrated in FIG. 1A-FIG. 2F.

In some embodiments, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen. Additionally, or alternatively, the cellular tumor sample is from a tumor previously determined to express of one or more of human TAP1 and/or human TAP2. In a specific embodiment, the tumor is human TAP1(+). In another specific embodiment, the tumor is human TAP2(+). In another specific embodiment, the tumor is human TAP1(−). In another specific embodiment, the tumor is human TAP2(−). In another specific embodiment, the tumor is human pan-TAP(+). In another specific embodiment, the tumor is human pan-TAP(−).

In a specific embodiment, the tumor is dMMR/human TAP1(+). In another specific embodiment, the tumor is dMMR/human TAP2(+). In another specific embodiment, the tumor is dMMR/human pan-TAP(+). In a specific embodiment, the tumor is dMMR/human TAP1(−). In another specific embodiment, the tumor is dMMR/human TAP2(−). In another specific embodiment, the tumor is dMMR/human pan-TAP(−). In a specific embodiment, the tumor is pMMR/human TAP1(+). In another specific embodiment, the tumor is pMMR/human TAP2(+). In another specific embodiment, the tumor is pMMR/human pan-TAP(+). In a specific embodiment, the tumor is pMMR/human TAP1(−). In another specific embodiment, the tumor is pMMR/human TAP2(−). In another specific embodiment, the tumor is pMMR/human pan-TAP(−).

In a specific embodiment, the tumor is MSI-H/human TAP1(+). In another specific embodiment, the tumor is MSI-H/human TAP2(+). In another specific embodiment, the tumor is MSI-H/human pan-TAP(+). In a specific embodiment, the tumor is MSI-H/human TAP1(−). In another specific embodiment, the tumor is MSI-H/human TAP2(−). In another specific embodiment, the tumor is MSI-H/human pan-TAP(−). In a specific embodiment, the tumor is MSI-L/human TAP1(+). In another specific embodiment, the tumor is MSI-L/human TAP2(+). In another specific embodiment, the tumor is MSI-L/human pan-TAP(+). In a specific embodiment, the tumor is MSI-L/human TAP1(−). In another specific embodiment, the tumor is MSI-L/human TAP2(−). In another specific embodiment, the tumor is MSI-L/human pan-TAP(−).

In a specific embodiment, the tumor is TMB-H/human TAP1(+). In another specific embodiment, the tumor is TMB-H/human TAP2(+). In another specific embodiment, the tumor is TMB-H/human pan-TAP(+). In a specific embodiment, the tumor is TMB-H/human TAP1(−). In another specific embodiment, the tumor is TMB-H/human TAP2(−). In another specific embodiment, the tumor is TMB-H/human pan-TAP(−). In a specific embodiment, the tumor is TMB-L/human TAP1(+). In another specific embodiment, the tumor is TMB-L/human TAP2(+). In another specific embodiment, the tumor is TMB-L/human pan-TAP(+). In a specific embodiment, the tumor is TMB-L/human TAP1(−). In another specific embodiment, the tumor is TMB-L/human TAP2(−). In another specific embodiment, the tumor is TMB-L/human pan-TAP(−).

Exemplary B2M antibodies that could be used in the present AHC/ACC methods are set forth in Table 4:

TABLE 4
Table of anti-human B2M antibodies.

| Vender | Clone ID | Clonality |
|---|---|---|
| Abcam | EP2978Y | Monoclonal |
| | EPR21752-214 | Monoclonal |
| | B2M/961 or rB2M/961 | Monoclonal |
| | B2M/1857R | Monoclonal |
| | B2M/1118 | Monoclonal |
| | D2E9 | Monoclonal |
| | HYB 003-01 | Monoclonal |
| Santa Cruz Biotechnology | BBM.1 | Monoclonal |
| | G-10 | Monoclonal |
| LSBio (LifeSpan) | LS-C195041 (Catalog | Polyclonal |
| | Number) | |
| | 2M2 | Monoclonal |
| Proteintech | 1C3B7 | Monoclonal |
| ThermoFisher | 246-E8.E7 | Monoclonal |
| | 3B12 | Monoclonal |
| | 3F2 | Monoclonal |
| | 4E12 | Monoclonal |
| | B2M-01 | Monoclonal |
| | B2M-02 | Monoclonal |
| | YTH470.5 | Monoclonal |
| Abnova | 3F9-2C2 | Monoclonal |
| | 6E11C11 | Monoclonal |
| | 7C3F9 | Monoclonal |
| | 9B1C7 | Monoclonal |
| | 2213 | Monoclonal |
| | 5F5B6 | Monoclonal |
| Novus Biologicals | 3G5H8 | Monoclonal |
| American Type Culture | 4c9 | Monoclonal |
| Collection (ATCC) | 1368 | Monoclonal |
| Creative Diagnostics | 578DU23.4.2 | Monoclonal |
| | 5H6B2 | Monoclonal |
| | C3N12 | Monoclonal |
| | E4B3 | Monoclonal |
| | JYD005-03 | Monoclonal |
| OriGene | OTI1A7 | Monoclonal |
| | OTI1A9 | Monoclonal |
| | TLD-3H12B | Monoclonal |
| Sigma-Aldrich | 3F9-2C2 | Monoclonal |
| Fisher Scientific | 6D585 | Monoclonal |
| | SPM374 | Monoclonal |
| VWR | B1G6 | Monoclonal |
| | C21 | Monoclonal |
| Cell Signaling Technology | D8P1H | Monoclonal |

### A.7. Multiplex AHC/ACC Methods

In another embodiment, the AHC or ACC assay is provided in a multiplex format. A multiplex AHC/ACC format involves affinity staining of multiple biomarkers in a single sample where at least some of the biomarkers are differentially labeled. Thus, for example, an IHC assay for 2 distinct biomarkers in the same sample, with a different chromogen or fluorophore for each biomarker would be considered a “multiplex IHC assay.” Likewise, an IHC assay for 3 biomarkers where 2 of the biomarkers are stained with the same chromogen or fluorophore and the 3rd biomarker is stained with a different chromogen or fluorophore would also be considered a “multiplex IHC assay.” The detectable moieties used in such a method should be compatible with multiplex affinity labeling methods, such as multiplex immunohistochemistry (mpIHC) or multiplex immunocytochemistry (mpICC).

In some embodiments, the detectable moieties are fluorophores. Exemplary fluorophores include several common chemical classes, such as coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11^{th} Edition. Exemplary fluorescent dyes compatible with mpIHC/mpICC and methodologies of using the same are disclosed at, for example, Gorris, Hofman, and Parra, each of which is incorporated by reference.

In other embodiments, the detectable moieties are molecules detectable via brightfield microscopy. Exemplary brightfield dyes compatible with multiplex IHC and methodologies of using the same are disclosed at, for example, Hofman, Ide, Morrison, Parra, Stack, and U.S. Pat. No. 10,041,950 B2, each of which is incorporated by reference. Specific examples include diaminobenzidine (DAB), 4-(dimethylamino) azobenzene-4′-sulfonamide (DABSYL), tetramethylrhodamine (TAMRA), N,N′-biscarboxypentyl-5,5′-disulfonato-indo-dicarbocyanine (Cy5), and Rhodamine 110 (Rhodamine).

In yet other embodiments, the detectable moieties are mass spectrometer-detectable labels. Reviews of mass spectrometry-based multiplexing methods and labels can be found at Levenson and Parra, for example.

In yet other embodiments, the detectable moieties are nucleic acid barcodes. As used in this context, a nucleic acid barcode is an oligonucleotide molecule conjugated to a biomarker-specific reagent in a manner that the oligonucleotide can be localized to a particular location in the sample. Exemplary nucleic acid barcode detection chemistry includes those used by PHENOCYCLER Technology from Akoya Biosystems, Inc. (specific oligonucleotides conjugated to biomarker-specific reagents or secondary detection reagents and complementary to fluorophore-conjugated reporter oligonucleotides), Digital Spatial Profiling (DSP) technology provided by Nanostring, Inc. (oligonucleotides conjugated to biomarker-specific reagents or secondary detection reagents via a photocleavable linker; upon cleavage of the linker, the oligonucleotide is identified, quantified, and mapped back to the tissue location), and INSITUPLEX technology from Ultivue, Inc. (specific oligonucleotides conjugated to biomarker-specific reagents or secondary detection reagents, which can be amplified to increase the ratio of barcodes per antibody and then bound to a complementary labeled reporter probe). See Tan.

A.7.a. Multiplex AHC/ACC Methods for Using Human HLA and/or B2M Biomarker-Specific Reagents

Multiplex AHC and multiplex ACC methods may be used for evaluating expression of MHC class I components in cellular tumor samples.

In one exemplary embodiment, a multiplex method is provided in which different human HLA markers (and optionally, human B2M) are differentially stained in the same sample. In a specific embodiment, a triplex AHC/ACC method is provided, wherein a single tissue sample is differentially stained for each of human HLA-A, HLA-B, and HLA-C. In another specific embodiment, a 4-plex AHC/ACC method is provided, wherein a single tissue sample is differentially stained for each of human HLA-A, HLA-B, HLA-C, and B2M. In another specific embodiment, a 2-plex AHC/ACC method is provided in which a single tissue sample is differentially stained with each of a human pan-HLA biomarker-specific reagent (or a human pan-HLA biomarker-specific reagent cocktail) and a human B2M biomarker-specific reagent. Such methods may be useful, for example, where it is desired to know which MHC class I components are expressed by the tumor without requiring use of multiple tumor samples.

In another exemplary embodiment, a multiplex method is provided in which a cellular tumor sample is differentially stained with a human tumor cell biomarker-specific reagent and either or both of at least one human HLA biomarker-specific reagent (such as a human HLA-A biomarker-specific reagent, a human HLA-B biomarker-specific reagent, or a human HLA-C biomarker-specific reagent (or combinations thereof), or a human pan-HLA biomarker-specific reagent) and a human B2M biomarker-specific reagent. The human tumor cell biomarker-specific reagent can be any biomarker-specific reagent that is useful for differentiating tumor cells from non-tumor cells in the same sample, such as the tumor differentiation markers reviewed by Painter et al., Useful Immunohistochemical Markers of Tumor Differentiation, Toxicological Pathology, 2010, Vol. 38, Issue 1, pp. 131-41. Exemplary human tumor cell biomarkers include cytokeratins or EPCAM (useful for epithelial tumors), vimentin (for mesenchymal tumors), and CD45 (for tumors of lymphoid lineage). Other markers specific to certain tumors include S100 and Melan-A for melanoma; uPA, hormone receptors, and HER2 for breast cancer; and specific recurrent genomic variants such as BRAF V600E or ALK fusions for tumors harboring those variants. In such an embodiment, the detectable moiety associated with the tumor cell biomarker-specific reagent should be distinguishable from the detectable moiety associated with the human HLA biomarker-specific reagent(s). Where multiple HLA biomarker-specific reagents are used, the different HLA biomarker-specific reagents may be labeled with the same detectable moiety (which is different from the detectable moiety associated with the human tumor cell biomarker-specific reagent). Alternatively, the different HLA biomarker-specific reagents may be differentially labeled from one another and from the human tumor cell biomarker-specific reagent.

In some embodiments of any of the foregoing multiplex AHC and multiplex ACC assays for human HLA and/or B2M, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen. Additionally, or alternatively, the cellular tumor sample is from a tumor previously determined to express of one or more of human TAP1 and/or human TAP2. In a specific embodiment, the tumor is human TAP1(+). In another specific embodiment, the tumor is human TAP2(+). In another specific embodiment, the tumor is human TAP1(−). In another specific embodiment, the tumor is human TAP2(−). In another specific embodiment, the tumor is human pan-TAP(+). In another specific embodiment, the tumor is human pan-TAP(−).

In a specific embodiment, the tumor is dMMR/human TAP1(+). In another specific embodiment, the tumor is dMMR/human TAP2(+). In another specific embodiment, the tumor is dMMR/human pan-TAP(+). In a specific embodiment, the tumor is dMMR/human TAP1(−). In another specific embodiment, the tumor is dMMR/human TAP2(−). In another specific embodiment, the tumor is dMMR/human pan-TAP(−). In a specific embodiment, the tumor is pMMR/human TAP1(+). In another specific embodiment, the tumor is pMMR/human TAP2(+). In another specific embodiment, the tumor is pMMR/human pan-TAP(+). In a specific embodiment, the tumor is pMMR/human TAP1(−). In another specific embodiment, the tumor is pMMR/human TAP2(−). In another specific embodiment, the tumor is pMMR/human pan-TAP(−).

In a specific embodiment, the tumor is MSI-H/human TAP1(+). In another specific embodiment, the tumor is MSI-H/human TAP2(+). In another specific embodiment, the tumor is MSI-H/human pan-TAP(+). In a specific embodiment, the tumor is MSI-H/human TAP1(−). In another specific embodiment, the tumor is MSI-H/human TAP2(−). In another specific embodiment, the tumor is MSI-H/human pan-TAP(−). In a specific embodiment, the tumor is MSI-L/human TAP1(+). In another specific embodiment, the tumor is MSI-L/human TAP2(+). In another specific embodiment, the tumor is MSI-L/human pan-TAP(+). In a specific embodiment, the tumor is MSI-L/human TAP1(−). In another specific embodiment, the tumor is MSI-L/human TAP2(−). In another specific embodiment, the tumor is MSI-L/human pan-TAP(−).

In a specific embodiment, the tumor is TMB-H/human TAP1(+). In another specific embodiment, the tumor is TMB-H/human TAP2(+). In another specific embodiment, the tumor is TMB-H/human pan-TAP(+). In a specific embodiment, the tumor is TMB-H/human TAP1(−). In another specific embodiment, the tumor is TMB-H/human TAP2(−). In another specific embodiment, the tumor is TMB-H/human pan-TAP(−). In a specific embodiment, the tumor is TMB-L/human TAP1(+). In another specific embodiment, the tumor is TMB-L/human TAP2(+). In another specific embodiment, the tumor is TMB-L/human pan-TAP(+). In a specific embodiment, the tumor is TMB-L/human TAP1(−). In another specific embodiment, the tumor is TMB-L/human TAP2(−). In another specific embodiment, the tumor is TMB-L/human pan-TAP(−).

A.7.b. Multiplex AHCIACC Methods Using Human TAPI and TAP2 Biomarker-Specific Reagents

Multiplex AHC and multiplex ACC methods may also be used for evaluating expression of human TAP components in cellular tumor samples.

In one exemplary embodiment, a multiplex method is provided in which different human TAP1 and human TAP2 are differentially stained in the same sample.

In some embodiments, a single tissue sample is differentially stained for each of human TAP1 and human TAP2. Such methods may be useful, for example, where it is desired to confirm that each of TAP1 and TAP2 are expressed or lost. Additionally, such methods components are expressed by the tumor without requiring use of multiple tumor samples.

In other embodiments, a multiplex method is provided in which a cellular tumor sample is differentially stained with a human tumor cell biomarker-specific reagent and at least one TAP biomarker-specific reagent (such as a human TAP1 biomarker-specific reagent, a human TAP2 biomarker-specific reagent, a human TAPI and a human TAP2 biomarker-specific reagent, a human pan-TAP biomarker-specific reagent). The human tumor cell biomarker-specific reagent can be any biomarker-specific reagent that is useful for differentiating tumor cells from non-tumor cells in the same sample, such as the tumor differentiation markers reviewed by Painter et al., Useful Immunohistochemical Markers of Tumor Differentiation, Toxicological Pathology, 2010, Vol. 38, Issue 1, pp. 131-41. Exemplary human tumor cell biomarkers include cytokeratins or EPCAM (useful for epithelial tumors), vimentin (for mesenchymal tumors), and CD45 (for tumors of lymphoid lineage). Other markers specific to certain tumors include S100 and Melan-A for melanoma; uPA, hormone receptors, and HER2 for breast cancer; CD30 or other lymphoid specific markers, and specific recurrent genomic variants such as BRAF V600E or ALK fusions for tumors harboring those variants. In such an embodiment, the detectable moiety associated with the tumor cell biomarker-specific reagent should be distinguishable from the detectable moiety associated with the human TAP biomarker-specific reagent(s). Where multiple human TAP biomarker-specific reagents are used, the different human TAP biomarker-specific reagents may be labeled with the same detectable moiety (which is different from the detectable moiety associated with the human tumor cell biomarker-specific reagent). Alternatively, the different TAP biomarker-specific reagents may be differentially labeled from one another and from the human tumor cell biomarker-specific reagent.

In some embodiments of any of the foregoing multiplex AHC and multiplex ACC assays for human TAP, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen. Additionally, or alternatively, the cellular tumor sample is from a tumor previously determined to express of one or more of human HLA-A, human HLA-B, and/or human HLA-C. In a specific embodiment, the tumor is HLA-A(+). In another specific embodiment, the tumor is HLA-B(+). In another specific embodiment, the tumor is HLA-C(+). In another specific embodiment, the tumor is pan-HLA(+). In a specific embodiment, the tumor is dMMR/HLA-A(+). In another specific embodiment, the tumor is dMMR/HLA-B(+). In another specific embodiment, the tumor is dMMR/HLA-C(+). In another specific embodiment, the tumor is dMMR/pan-HLA(+). In a specific embodiment, the tumor is pMMR/HLA-A(+). In another specific embodiment, the tumor is pMMR/HLA-B(+). In another specific embodiment, the tumor is pMMR/HLA-C(+). In another specific embodiment, the tumor is pMMR/pan-HLA(+). In a specific embodiment, the tumor is MSI-H/HLA-A(+). In another specific embodiment, the tumor is MSI-H/HLA-B(+). In another specific embodiment, the tumor is MSI-H/HLA-C(+). In another specific embodiment, the tumor is MSI-H/pan-HLA(+). In a specific embodiment, the tumor is MSI-L/HLA-A(+). In another specific embodiment, the tumor is MSI-L/HLA-B(+). In another specific embodiment, the tumor is MSI-L/HLA-C(+). In another specific embodiment, the tumor is MSI-L/pan-HLA(+). In a specific embodiment, the tumor is TMB-H/HLA-A(+). In another specific embodiment, the tumor is TMB-H/HLA-B(+). In another specific embodiment, the tumor is TMB-H/HLA-C(+). In another specific embodiment, the tumor is TMB-H/pan-HLA(+). In a specific embodiment, the tumor is TMB-L/HLA-A(+). In another specific embodiment, the tumor is TMB-L/HLA-B(+). In another specific embodiment, the tumor is TMB-L/HLA-C(+). In another specific embodiment, the tumor is TMB-L/pan-HLA(+).

A.7.c. Multiplex AHC/ACC Methods for Human TAP and Human MHC Class I Component(s)

Multiplex AHC and multiplex ACC methods also may be used for evaluating co-expression of human TAP components and human MHC class I components in the same cellular tumor samples.

In one exemplary embodiment, a multiplex method is provided in which a cellular sample is differentially stained with (a) at least 1 human TAP biomarker-specific reagent (including, for example, a human TAP1 biomarker-specific reagent, a human TAP2 biomarker-specific reagent, both a human TAPI and a human TAP2 biomarker-specific reagent, a human pan-TAP biomarker-specific reagent); and (b) either or both of: (b1) at least 1 human HLA biomarker-specific reagent (including, for example, a human TAP1 biomarker-specific reagent, a human TAP2 biomarker-specific reagent, both a human TAP1 and a human TAP2 biomarker-specific reagent, a human pan-TAP biomarker-specific reagent), and (b2) at least one human B2M biomarker-specific reagent. The sample may also optionally be differentially stained with a human tumor cell biomarker-specific reagent.

The human tumor cell biomarker-specific reagent can be any biomarker-specific reagent that is useful for differentiating tumor cells from non-tumor cells in the same sample, such as the tumor differentiation markers reviewed by Painter et al., Useful Immunohistochemical Markers of Tumor Differentiation, Toxicological Pathology, 2010, Vol. 38, Issue 1, pp. 131-41. Exemplary human tumor cell biomarkers include cytokeratins or EPCAM (useful for epithelial tumors), vimentin or other tumor specific marker such as S100 for melanoma (for mesenchymal tumors), and CD45 (for tumors of lymphoid lineage). In such an embodiment, the detectable moiety associated with the tumor cell biomarker-specific reagent should be distinguishable from the detectable moiety associated with the human TAP biomarker-specific reagent(s) and the human HLA and/or.

In some embodiments of any of the foregoing multiplex AHC and multiplex ACC assays for human TAP, the cellular tumor sample may be from a tumor that has previously been screened by a tumor mutational screen, such as a tumor mutational burden screen, a microsatellite screen, and/or a mismatch repair screen. In an exemplary embodiment, the cellular tumor sample is from a tumor previously determined to be a pMMR tumor, an MSI-L/MSS tumor, and/or a TMB-L tumor. In another exemplary embodiment, the cellular tumor sample is from a tumor previously determined to be a dMMR tumor, an MSI-H tumor, and/or a TMB-H tumor. In other exemplary embodiments, the cellular tumor sample has not been screened by a tumor mutational screen, such as a tumor mutational burden screen, a microsatellite screen, and/or a mismatch repair screen.

### A.8. Staining Assessment/Scoring

Subsequent to staining, the obtained biological sample may be evaluated for the expression of the constituent elements of the TAP complex and/or the MHC-I complex. Since normal tissues would be expected to express both functional MHC-I machinery and functional TAP machinery, the samples are evaluated for loss of expression of the components of the APM within a tumor cells. Specific staining is distinguished from non-specific staining and the extent to which specific staining is lost in a tumor region is recorded.

A.8.a. Specific Versus Non-Specific Staining

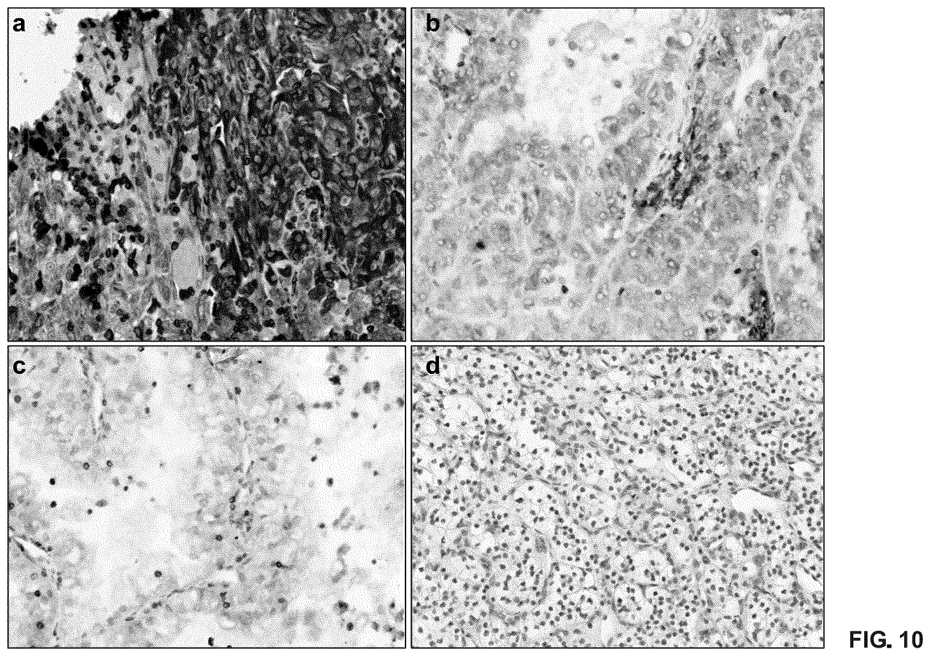

Specific TAP expression is cytoplasmic, generally diffuse with some cases displaying a finely granular quality. Nuclear staining is considered non-specific and may be ignored. Any cell or region within the sample that has a cytoplasmic staining pattern above background levels is considered to be TAP-positive and any cell or region within the sample that lacks a cytoplasmic staining pattern above background is considered TAP negative (see FIG. 10).

Specific HLA and B2M expression is membranous, and can have a discontinuous, circumferential, or basolateral pattern. Any cell or region within the sample that has a discontinuous, circumferential, or basolateral staining pattern above background levels is considered to be HLA- or B2M-positive and any cell or region within the sample that lacks such a staining pattern above background is considered HLA- or B2M-negative.

A.8.b. Manual Assessment

In some embodiments, the extent of loss of specific staining is determined manually evaluated.

As one example, a trained user (such as a pathologist) may visually inspect the stained sample (such as under a microscope), identify one or more tumor region(s) of interest (ROI), and estimate the extent of specific staining within the tumor ROI(s). The extent of loss is the estimated percentage of the area of the tumor ROI(s) that lacks specific staining.

As another example, the relative intensity may be taken into consideration. For example, a trained user (such as a pathologist) may visually inspect the stained sample (such as under a microscope), identify one or more tumor region of interest (ROI), and identify any areas of the tumor ROI that have reduced specific staining intensity relative to the surrounding stroma. The extent of loss is the estimated percentage of the area of the tumor ROI with reduced staining relative to the surrounding stroma.

As another example, the relative intensity is considered by applying a 0-3+ scale. In such an embodiment, the AHC/ACC staining method is optimized such that specific staining occurs across a range of intensities roughly correlating to the expression level of the biomarker of interest. The different stain intensity levels are categorized as 0, 1+, 2+, or 3+ with “O” having substantially no detectable specific staining, “1+” having a weak specific staining intensity, “2+” having a moderate specific staining intensity, and “3+” having strong specific staining intensity. The extent of loss in such an example may be estimated in a number of different ways. A pre-determined intensity cutoff may be selected (such as a cutoff at intensity level of 1) and the extent of loss is the estimated percentage of the area of the tumor ROI with a staining intensity below the pre-selected cutoff. Alternatively, a composite “score” may be quantified that incorporates the extent of staining at each intensity level, such as an H-score: h-score=[1×(% cells at 1+)+2×(% cells at 2+)+3×(% cells at 3+). In such an embodiment, the extent of loss=300-H-score. Thus, for example, where 10% of the tumor ROI is at 0 intensity, 20% of the tumor ROI is at 1+, 30% of the ROI is at 2+, and 40% of the tumor ROI is at 3+, then

$$H - score = ( 0 \times 10 ) + ( 1 \times 20 ) + ( 2 \times 30 ) + ( 3 \times 40 ) = 200 ; and$$

$$Extent loss = 300 - 200 = 100.$$

Another example of a composite score is the Allred Score, which combines (a) the percentage of positive cells and (b) the intensity of the predominant reaction product in the tumor ROI. An exemplary Allred scoring system is illustrated at Table 5:

TABLE 5

| Positive Cells | Proportion | | Intensity |
|---|---|---|---|
| (%) | Score (PS) | Intensity | Score (IS) |
| 0 | 0 | None | 0 |
| <1 | 1 | Weak | 1 |
| 1-10 | 2 | Intermediate | 2 |
| 11-33 | 3 | Strong | 3 |

| | | |
|---|---|---|
| 34-66 | 4 | Allred Score (AS) = PS + IS |

| | |
|---|---|
| ≥67 | 5 |

Since 8 is the maximum possible Allred score, the extent of loss in such an example=8−AS. Thus, for example, where 10% of the tumor ROI is at 0 intensity, 20% of the tumor ROI is at 1+, 30% of the ROI is at 2+, and 40% of the tumor ROI is at 3+, then: Positive Cell %=90, so PS=5; the predominate intensity is 3+, so IS=3; AS=5+3=8; and Extent of Loss=8−8 =0.

In a specific embodiment, a simplex TAP AHC/ACC assay using a brightfield dye and optimized for manually scoring on a 0-3+ scale according to the Table 6 is performed and at least the extent of specific staining is estimated, optionally including consideration of staining intensity as described herein. The extent of loss is then determined by any of the methods set forth in this section.

TABLE 6
TAP Stain Intensity Criteria

| Stain Intensity Score | |
|---|---|
| (0-3 with 0.25 increments) | Criteria |
| 0 | Absence of specific cytoplasmic signal |
| 1 | WEAK cytoplasmic staining of the cells |
| 2 | MODERATE cytoplasmic staining of the cells |
| 3 | STRONG cytoplasmic staining of the cells |
| Not Evaluable (N/E) | Interpretation is not possible, e.g., no tissue/tumor |
| | present, artifacts, or edge artifacts, or insufficient |
| | staining of normal cells (i.e., negative internal controls) |

In a specific embodiment, the simplex TAP assay is an IHC assay optimized for manually scoring on a 0-3+ scale as described in Table 6, wherein the sample is a formalin-fixed, paraffin embedded tissue section, and the extent of loss is determined by evaluating the percentage of a tumor ROI having a stain intensity of less than 1+.

In another specific embodiment, an HLA or B2M AHC/ACC assay using a brightfield dye and optimized for manually scoring on a 0-3+ scale according to the Table 7 is performed and at least the extent of specific staining is estimated, optionally including consideration of staining intensity as described herein. The extent of loss is then determined by any of the methods set forth in this section.

TABLE 7

| Stain Intensity Score | Criteria |
|---|---|
| 0 | Absence of specific membrane signal |
| 1 | WEAK membrane staining of the cells |
| 2 | MODERATE membrane staining of the cells |
| 3 | STRONG membrane staining of the cells |
| Not Evaluable (N/E) | Interpretation is not possible, e.g., no tissue/tumor |
| | present, artifacts, or edge artifacts |

In a specific embodiment, the simplex HLA or B2M assay is an IHC assay optimized for manually scoring on a 0-3+ scale as described in Table 5, wherein the sample is a formalin-fixed, paraffin embedded tissue section, and the extent of loss is determined by evaluating the percentage of a tumor ROI having a stain intensity of less than 1+.

Where the assay is a TAP/MHC multiplex AHC/ACC assay, the expression of TAP may be evaluated separately from the expression of the MHC component(s), or they may be considered in combination. When considered separately, the extent of loss of TAP expression and the extent of loss of MHC expression may be evaluated essentially as described above. When considered together, the sample may be evaluated for co-localization of the stain associated with TAP and stain associated with the MHC component(s). In such an embodiment, each portion of the tumor ROI can be categorized as having one of the patterns set forth in Table 8:

TABLE 8

| Pattern | Criteria |
|---|---|
| TAP_{High}/MHC_{High} | Any level of specific cytoplasmic staining above |
| | background of the TAP-associated stain with any |
| | level of specific membrane staining above |
| | background of the MHC-associated stain |
| TAP_{Low}/MHC_{High} | Absence of specific cytoplasmic staining above |
| | background of the TAP-associated stain with any |
| | level of specific membrane staining above |
| | background of the MHC-associated stain |
| TAP_{High}/MHC_{Low} | Any level of specific cytoplasmic staining above |
| | background of the TAP-associated stain with |
| | absence of specific membrane staining above |
| | background of the MHC-associated stain |
| TAP_{Low}/MHC_{Low} | Absence of specific cytoplasmic staining above |
| | background of the TAP-associated stain and |
| | absence of specific membrane staining above |
| | background of the MHC-associated stain |

In such an embodiment, the sample can be categorized based on MHC and TAP loss in a number of ways. For example, the sample may be categorized according to the predominate staining pattern observed. Thus, for example, where about 40% of the sample is TAP_{Low}/MHC_{High}, about 30% of the sample is TAP_{High}/MHC_{Low}, about 20% of the sample is TAP_{Low}/MHC_{Low}, and about 10% of the sample is TAP_{High}/MHC_{High}, the sample may be categorized as TAP_{Low}/MHC_{High}. As another example, the TAP_{High}/MHC_{Low} and TAP_{Low}/MHC_{Low} may be considered together under the category “MHC_{Low}.” In such an embodiment, the predominate pattern would be “MHC_{Low},” as about 50% of the sample is either TAP_{High}/MHC_{Low} or TAP_{Low}/MHC_{Low}. In yet another example, the extent of MHC loss and the extent of TAP loss may be separately extrapolated from the relative amounts of the observed staining patterns. Thus, for example, the MHC loss would be about 30% (sum of TAP_{Low}/MHC_{Low} and TAP_{High}/MHC_{Low}) and the extent of TAP loss would be about 60% (sum of TAP_{Low}/MHC_{High} and TAP_{Low}/MHC_{Low}). In yet another embodiment, the evaluation of TAP expression may only be conducted in MHC_{High} regions. In such an embodiment, the extent of the sample having a loss of MHC specific staining is estimated. Then, in the regions in which there is observable MHC specific staining, the extent of TAP_{High}/MHC_{High} and TAP_{Low}/MHC_{Low} staining is evaluated. The predominant staining pattern may be assigned to the sample. Thus, where the sample is about 30% MHC_{Low}, about 40% TAP_{Low}/MHC_{High}, and about 30% TAP_{High}/MHC_{High}, the sample may be categorized as TAP_{Low}/MHC_{High}.

In another specific embodiment, a TAP/MHC duplex assay is provided, wherein TAP is stained with a first brightfield dye and the MHC complex is stained with a second brightfield dye, wherein the duplex assay is optimized for manually scoring each of TAP and MHC on a 0-3+ scale according to the Table 6 and Table 7, and at least the extent of specific staining is estimated, optionally including consideration of staining intensity and/or duplex staining patterns as described herein. The extent of loss is then determined by any of the methods set forth in this section.

A.8.c. Assessment Using a Digital Pathology System

As another example, the stained sample may be evaluated using a digital pathology system. There are two basic components of digital pathology systems: (1) a scanning or image acquisition system for generating digital images of a stained sample; and (2) an image analysis system for identifying and quantifying specific features within the generated digital images.

An image acquisition system may include a scanning platform such as a slide scanner that can scan the stained slides at 20×, 40×, or other magnifications to produce high resolution whole-slide digital images, including for example slide scanners. In some embodiments, a slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. In some embodiments, digital data at a number of different X-Y locations (and in some cases, at multiple Z planes) on the slide are captured by the camera's charge-coupled device (CCD), and the images are joined together to form a composite image of the entire scanned surface. Common methods to accomplish this include: (1) Tile based scanning, in which the slide stage or the optics are moved in very small increments to capture square image frames, which overlap adjacent squares to a slight degree. In some embodiments, the captured squares are then automatically matched to one another to build the composite image; and (2) Line-based scanning, in which the slide stage moves in a single axis during acquisition to capture a number of composite image “strips.” In some embodiments, the image strips can then be matched with one another to form the larger composite image.

A detailed overview of various scanners (both fluorescent and brightfield) can be found at Farahani et al., Whole slide imaging in pathology: advantages, limitations, and emerging perspectives, Pathology and Laboratory Medicine Int'l, Vol. 7, p. 23-33 (June 2015), the disclosure of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000×T, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600×; and Zeiss AXIO SCAN.Z1. In some embodiments, the scanning device is a digital pathology device as disclosed any of U.S. Pat. No. 9,575,301; U.S. Patent Application Publication No. 2014/0178169; U.S. Pat. No. 9,575,301; U.S. Patent Application Publication No. 2014/0178169; United States Patent Publication Nos. 2021/0092308; and/or U.S. Patent Application Publication No. 2021/0088769, the content of each of which is incorporated by reference in its entirety.

Exemplary commercially available image analysis software packages include VENTANA VIRTUOSO software suite (Ventana Medical Systems, Inc.); TISSUE STUDIO, DEVELOPER XD, and IMAGE MINER software suites (Definiens); BIOTOPIX, ONCOTOPIX, and STEREOTOPIX software suites (Visiopharm); and the HALO platform (Indica Labs, Inc.).

A sample stained as described herein is imaged on a scanner system to generate a high-quality digital image of the stained sample. The digital image is then analyzed by the image analysis system to identify and classify one or more relevant objects in the sample. For example, the image analysis may identify all cells in a tumor ROI and then classify the cells as either biomarker-positive or biomarker-negative. As another example, the image analysis may differentiate tumor cells from other cells in the tumor ROI and then classify the tumor cells as either biomarker-positive or biomarker-negative. The number and/or percentage of each classification of cell may then be reported. Additionally, or alternatively, the image analysis system may plot the density of biomarker-positive and biomarker-negative cells onto an image of the tumor ROI and identify regions of the ROI that are biomarker-positive (i.e., regions having a pre-determined density of biomarker-positive cells) and regions of the ROI that are biomarker-negative (i.e., regions having a pre-determined density of biomarker-negative cells). The image analysis system may also take into consideration the intensity of the biomarker stain and thus may further categorize individual cells and/or regions of the sample on the basis of intensity. As one exemplary embodiment, the image analysis system may further stratify biomarker-positive regions of the tumor ROI into “high,” “medium,” and “low” expressing regions.

Where the sample has been stained in a brightfield multiplex format, the image analysis system may also perform a deconvolution or color separation process on the digital image. Deconvolution essentially separates an image having a stain mixture into the contribution of the individual single stains allowing the individual stain components to be evaluated separately. By applying deconvolution methods, individual cells and/or regions of the sample can be categorized on the basis of multiple biomarkers. Thus, for example, in an MHC/TAP duplex, each tumor cell may be categorized on the basis of both MHC-associated stain and the TAP-associated stain (such as by marking the cell as MHC_{High} or MHC_{Low} and TAP_{High} or TAP_{Low}). In an MHC/DM duplex or a TAP/DM duplex, each tumor cell may be automatically identified on the basis of the differentiation marker stain and categorized on the basis of the MHC-associated stain or the TAP-associated stain. In an MHC/TAP/DM triplex, each tumor cell may be automatically identified on the basis of the differentiation marker stain and categorized on the basis of each of the MHC-associated stain and the TAP-associated stain. Exemplary brightfield deconvolution methods are disclosed at, for example in PCT/EP2015/061226, PCT/EP2015/067384, PCT/EP2016/081329, PCT/EP2018/070956, the disclosures of which are hereby incorporated by reference herein in their entireties.

The image analysis system may also perform a registration function between digital images of serial sections. A registration function basically matches tissue features shared between serial sections so that the images may be overlaid with one another. In this way the same regions of the tissue may be evaluated for multiple biomarkers without requiring co-staining. For example, a digital image of an MHC-stained tumor section may be registered to a TAP-stained serial section. Additionally, or alternatively, the registration function enables a biomarker-stained section (such as an MHC-stained and/or TAP-stained section) to be matched to and overlaid with a morphologically stained serial section (such as a hematoxylin and eosin stained serial section). In this way, a tumor region could be annotated in the morphologically stained section and then registered to the biomarker section for analysis.

### B. Flow Cytometry Assays

Flow cytometry may be utilized to assess a percentage of TAP positive tumor cells and TAP positive immune cells in a cellular tumor sample. Flow cytometry is a technique used to identify and distinguish between different particle types (e.g., cell types) present in a fluid medium. In general, when flow cytometrically analyzing a sample, an aliquot of the sample is first introduced into a flow path of a flow cytometer where each of the particles is exposed individually to one or more sources of light, such as one or more sources of light having differing wavelengths. Various parameters (e.g., light scatter, fluorescent emission, etc.) may be measured for each particle and subsequently evaluated as described herein.

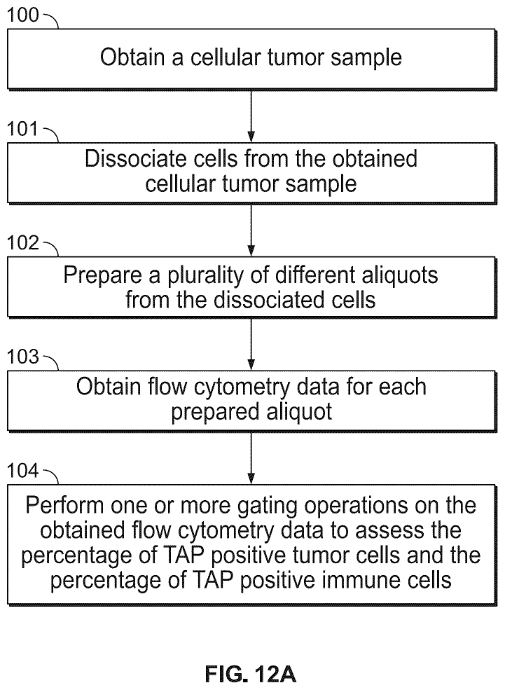

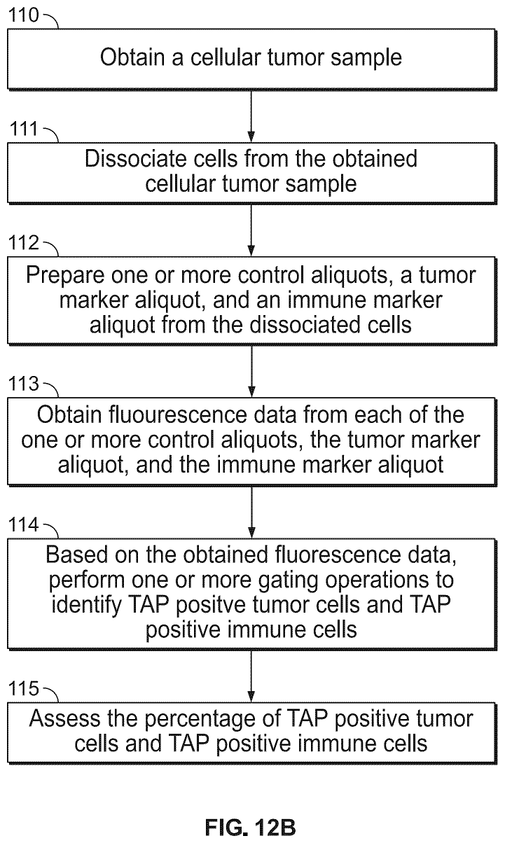

With reference to FIGS. 12A and 12B, in some embodiments, cells within an obtained cellular tumor sample (steps 100 and 110) are first dissociated from the cellular tumor sample (steps 101 and 111). For instance, cells may be dissociated from the cellular tumor sample through mechanical shearing and/or by subjecting the sample to one or more chemical or biochemical reagents.

Once the cells have been dissociated, one or more control aliquots, a tumor marker aliquot, and an immune marker aliquot are prepared (steps 102 and 112). In general, the aliquots are prepared by (i) staining the dissociated cells within the aliquots for the presence of one or more biomarkers; and/or (ii) incubating the dissociated cells within the aliquots with one or more detection reagents (e.g., secondary antibodies conjugated to a detectable label, such as a fluorescent label).

In some embodiments, a tumor marker aliquot may be prepared (steps 102 or 112) by staining dissociated cells for the presence of (i) a tumor cell biomarker, and (ii) one or more TAP biomarkers (e.g., TAP1 and/or TAP2). In some embodiments, the tumor cell biomarker is selected from a specific cytokeratin marker, pan-cytokeratin, EPCAM, Aneuploid DNA or other DNA content visualized by DAPI or similar DNA stain, S100, Melan-A, GPC-3, specific tumor genomic alterations such as BRAF V600E, KRAS mutation, or ALK fusion, HER2, uPA, and hormone receptors such as ER or PR. For instance, a tumor cell marker (e.g., CK 8/18) may be stained with a first fluorescent label; while a TAP biomarker (e.g., TAP1) may be stained with a second fluorescent label, where the first and second fluorescent labels are different (e.g., a first fluorescent label may have a first wavelength, while a second fluorescent label may have a second wavelength).

In some embodiments, an immune marker aliquot may be prepared (steps 102 or 112) by staining dissociated cells for the presence of (i) an immune cell marker; and (ii) one or more TAP biomarkers (e.g., TAP1 and/or TAP2). In some embodiments, the immune cell marker is selected from CD45, CD3, CD4, CD8, CD20, CD 25, CD19, CD163, CD68, CD69 and CD103. For instance, an immune cell marker (e.g., CD45) may be stained with a first fluorescent label; while a TAP biomarker (e.g., TAP1) may be stained with a second fluorescent label, where the first and second fluorescent labels are different.

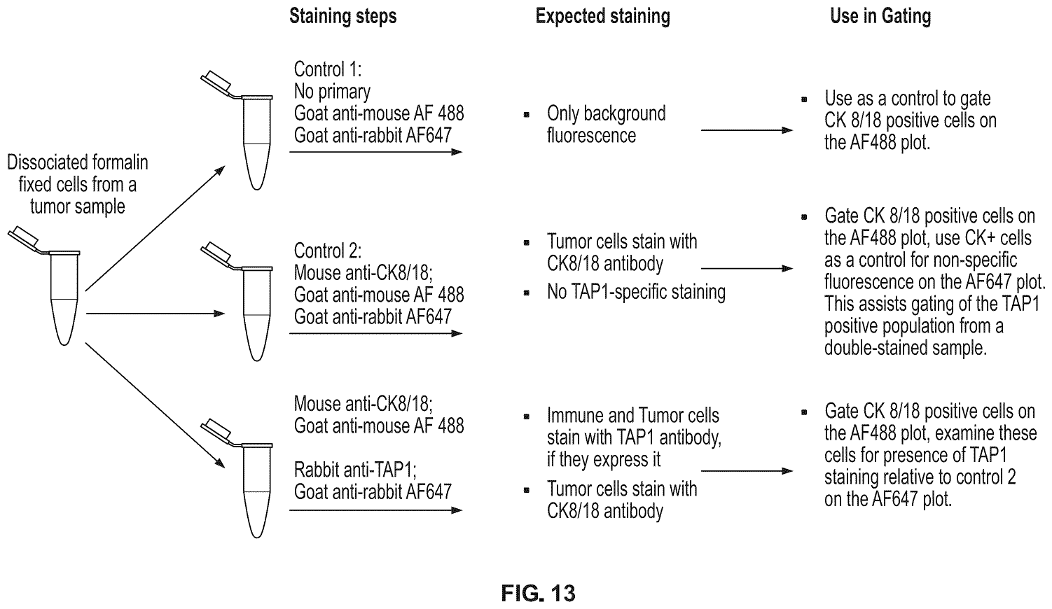

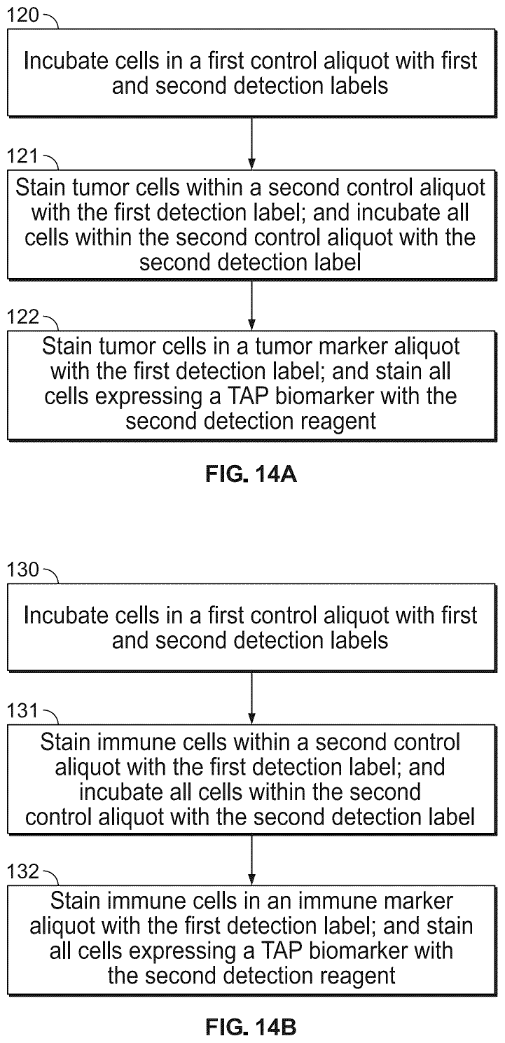

In some embodiments, one or more control aliquots may be prepared (steps 102 or 112). In some embodiments, a single control aliquot is prepared. In other embodiments, two or more control aliquots are prepared. By way of example, FIGS. 13 and 14A illustrate methods of preparing a tumor marker aliquot and two control aliquots. Likewise, FIG. 14B depicts a method of preparing an immune marker aliquot and two respective control samples.

In some embodiments, a first control aliquot may be prepared by incubating dissociated cells with the detection reagents used to label (i) a tumor cell biomarker or an immune cell biomarker; and (ii) a TAP biomarker. In this way, the first control aliquot may be used as a control for background staining (e.g., background fluorescence). For instance, dissociated cells within a first control aliquot may be incubated with (i) a first secondary antibody conjugated to a first fluorescent label; and (ii) a second secondary antibody conjugated to a second fluorescent label, where the first and second fluorescent labels are different; and where the first fluorescent label is used to label either the tumor cell biomarker or the immune cell biomarker (in the tumor marker and immune marker aliquots, respectively); and where the second fluorescent label is used to label the TAP biomarker.

By way of example, if, in a tumor marker aliquot, CK8/18 (+) tumor cells are labeled with AF488 and TAP1(+) tumor cells are labeled with AF647, then dissociated cells within a first control aliquot may be incubated with (i) a first secondary antibody conjugated to a AF488 label; and (ii) a second secondary antibody conjugated to a AF647 label (see FIGS. 13 and 14A). By way of another example, if, in an immune marker aliquot, CD45 (+) immune cells are labeled with AF488 and TAP1(+) immune cells are labeled with AF647, then dissociated cells within a first control aliquot may be incubated with (i) a first secondary antibody conjugated to a AF488 label; and (ii) a second secondary antibody conjugated to a AF647 label (see FIG. 14B).

In other embodiments, a second control aliquot may be prepared by (i) staining dissociated cells for the presence of either a tumor cell biomarker or an immune cell biomarker with a first fluorescent label; and (ii) incubating the dissociated cells with a secondary antibody conjugated to a second fluorescent label, where the first and second fluorescent labels are different. In this way, the second control aliquot may serve to identify either tumor cell marker positive or immune cell marker positive cells with a first label while accounting for non-specific staining (e.g., fluorescence) of the second label.

By way of example, if, in a tumor marker aliquot, CK8/18 (+) tumor cells are labeled with AF488 and TAP1(+) tumor cells are labeled with AF647, then dissociated cells within a second control aliquot may be (i) stained for the presence of the tumor cell biomarker with the AF488 label; and (ii) incubated with a secondary antibody conjugated to the AF647 label (see FIGS. 13 and 14A). By way of another example, if, in an immune marker aliquot, CD45 (+) immune cells are labeled with AF488 and TAP1(+) immune cells are labeled with AF647, then dissociated cells within a second control aliquot may be (i) stained for the presence of the immune cell biomarker with the AF488 label; and (ii) incubated with a secondary antibody conjugated to the AF647 label

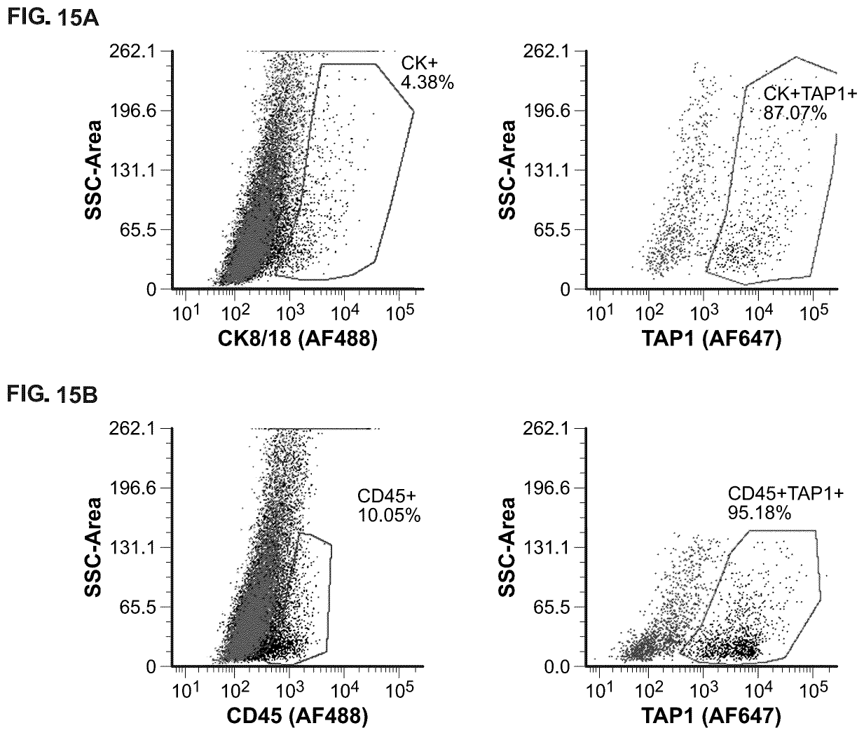

Following the preparation of the various aliquots, fluorescence data may be generated from each of the one or more control aliquots, the tumor marker aliquot, and the immune marker aliquot (steps 103 and 113) using a flow cytometer. In some embodiments, the flow cytometry data generated may be plotted as one or more scatter plots (see, e.g., FIGS. 15A and 15B). In some embodiments, a scatter plot may be divided into one or more regions or “gates.” As used herein, the term “gating” refers to the selection of a population of particles from a sample, where the selection is made based on the characteristics (e.g., forward scattering content (FSC), side scattering content (SSC), and/or fluorescence intensity) of the particles within the sample. To select an appropriate gate, in some embodiments, the flow cytometry data is plotted so as to obtain appropriate separation of subpopulations of particles, e.g., by adjusting the configuration of the instrument, including, for example, excitation parameters, collection parameters, compensation parameters, etc. In some embodiments, particles with the required characteristics will “pass through” the gate and are selected for further analysis, while those that do not have the required characteristics will be excluded from further analysis. Notably, the process of gating does not change the data, i.e., it only depicts the data in a way that flow cytometry analysts find themselves familiar with.

Based on the obtained fluorescence data obtained at steps 103 and 113, one or more gating operations may be performed to identify TAP positive tumor cells and/or TAP positive immune cells (steps 104, 114). For example, FIGS. 15A and 15B depict methods of quantifying TAP1 positive tumor and TAPI positive immune cells in a cellular tumor sample. Specifically, FIG. 15A illustrates a method of performing a sequential gating operation, namely a first gating operation to identify tumor cell marker positive cells, and then performing a second gating operation to identify TAP1 positive tumor cells within the tumor cell marker position cell population. Similarly, FIG. 15B illustrates a method of performing a sequential gating operation to identify immune cell marker positive cells followed by identifying TAP1 positive immune cells within the immune cell marker position cell population.

### C. RNAseq Assays

Evaluating gene expression and identifying transcripts that are differentially expressed between two conditions in a cell, tissue, or organism is an important approach to deciphering the molecular physiology of the cell. A recently developed technique called RNA Sequencing (RNA-Seq) uses massively parallel sequencing to allow transcriptome analyses of genomes at a far higher resolution than is available with Sanger sequencing- and microarray-based methods. In the RNA-Seq method, complementary DNAs (cDNAs) generated from the RNA of interest are directly sequenced using next-generation sequencing technologies. More specifically, RNA-seq involves isolation of total RNA from tissues or cells of interest followed by the construction of DNA libraries and sequencing of these libraries using a next-generation sequencing instrument. In some cases, specific species of RNA may be depleted (ribosomal RNA) or enriched (poly A-based or size-based selection) prior to conversion to cDNA. The reads obtained from this can then be aligned to a reference genome in order to construct a whole-genome transcriptome map (see Examples 4 and 5, herein).

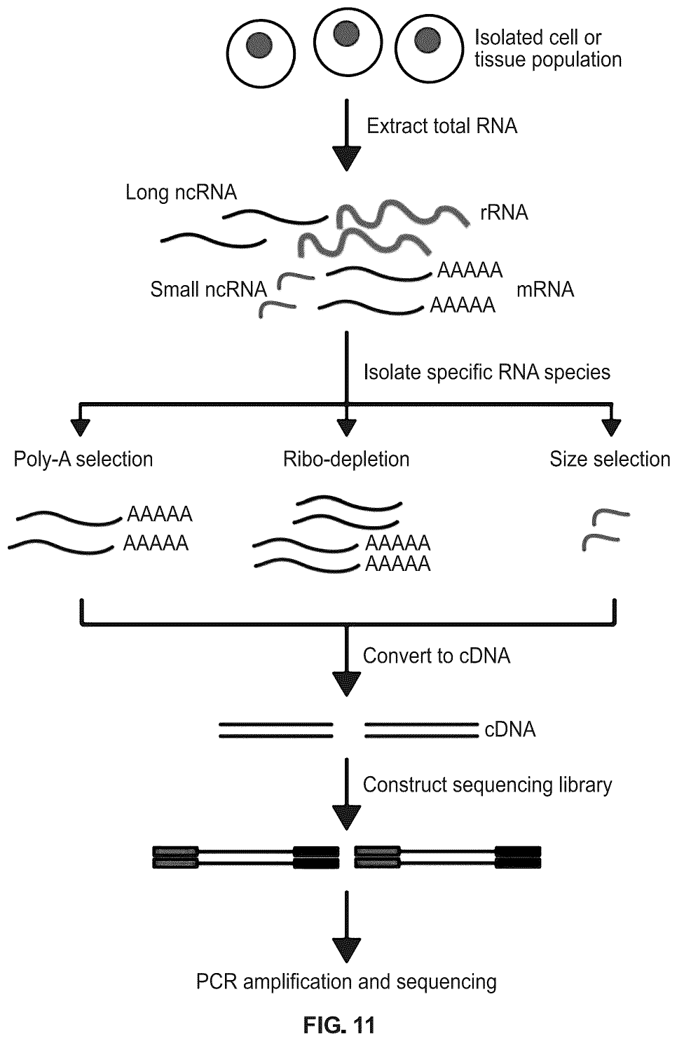

An exemplary method of performing an RNAseq analysis, such as on sample derived from a cellular tissue sample, is illustrated in FIG. 11. First, RNA is extracted from the cellular tissue sample. Next, subsets of RNA molecules are isolated using a specific protocol, such as a poly-A selection protocol to enrich for polyadenylated transcripts or a ribo-depletion protocol to remove ribosomal RNAs. Subsequently, the RNA is converted to complementary DNA (cDNA) by reverse transcription and sequencing adaptors are ligated to the ends of the cDNA fragments. (Kukurba K R, Montgomery S B. RNA Sequencing and Analysis. Cold Spring Harb Protoc. April 13; 2015 (11): 951-69.doi: 10.1101/pdb.top084970. PMID: 25870306; PMCID: PMC4863231.) Following amplification (such as by PCR), the RNA-Seq library may be sequenced, such as with a next-generations sequencing technique. As used herein, the term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Examples of next generation sequencing techniques include, but are not limited to, sequencing by synthesis, sequencing by ligation, and sequencing by hybridization.

### Diagnostic Workflows and Methods of Treatment Using Tap & MHC-I

The assessment of TAP and MHC-I component expression is useful for identifying those tumors that would benefit from a mutational screen for prediction of a response to an MHC-I-directed therapeutic. Additionally, the assessment of TAP and MHC-I component expression (optionally when combined with consideration of mutational screen) is useful for identifying those tumors that would benefit from administration of an MHC-I-directed therapeutic. Exemplary workflows for making such decisions are disclosed herein at FIG. 1A-1C and FIG. 2A-2F.

In this context, the tumor is categorized on the basis of the extent of loss of MHC expression and the extent of loss of TAP expression. To this end, stratification cutoffs for each of MHC and TAP are selected to stratify responders from non-responders to an MHC-I-dependent immunotherapeutic agent. For example, one or more stratification cutoffs may be selected to separate the patients into bins according to the ranking of the extent of MHC and TAP loss compared to the population (such as, for example, a quartile, decile, or percentile ranking of the extent of MHC and/or TAP loss or the extent of a particular staining pattern) or the likelihood of an event occurring (such as likely responder versus unlikely responder, or etc.). In one example, stratification cutoffs are selected using receiver operator characteristic (ROC) curves. ROC curves allow users to balance the sensitivity of a model (i.e., prioritize capturing as many “positive” or “likely to respond” candidates as possible) with the specificity of the model (i.e., minimizing false positives for “likely to respond” candidates). In an embodiment, a cutoff is selected between likely to respond and unlikely to respond risk bins. In yet another embodiment, the cutoff may be a mean or median score (such as the mean or median extent of MHC and/or TAP loss or the mean or median extent of a particular staining pattern observed). Cutoff analysis may be performed using a computerized statistical analysis software suite (such as The R Project for Statistical Computing (r-project.org), SAS, MATLAB, among others).

In an exemplary embodiment, the MHC stratification cutoff is selected such that tumors having an extent of MHC loss that exceeds the stratification cutoff are unlikely to respond to the MHC-I-dependent immunotherapeutic agent (regardless of TAP status or mutational screen status), while tumors having an extent of MHC loss that falls below the stratification cutoff are likely to respond (based upon either the TAP status or the mutational screen status). In an exemplary embodiment, the MHC stratification cutoff is selected to differentiate between dMMR, MSI-H, and/or TMB-H tumors that are likely to respond from dMMR, MSI-H, and/or TMB-H tumors that are unlikely to respond. In another exemplary embodiment, the MHC stratification cutoff is selected to differentiate between TAP(−) tumors that are likely to respond from TAP(−) tumors that are unlikely to respond. In another exemplary embodiment, the MHC stratification cutoff is selected to differentiate between TAP(+)/dMMR, TAP(+)/MSI-H, and/or TAP(+)/TMB-H tumors that are likely to respond from TAP(+)/dMMR, TAP(+)/MSI-H, and/or TAP(+)/TMB-H tumors that are unlikely to respond. In another exemplary embodiment, the MHC stratification cutoff is selected to differentiate between TAP(−)/pMMR, TAP(−)/MSI-L, and/or TAP(−)/TMB-L tumors that are likely to respond from TAP(−)/pMMR, TAP(−)/MSI-L, and/or TAP(−)/TMB-L tumors that are unlikely to respond.

In another exemplary embodiment, the TAP stratification cutoff is selected such that MHC(+) tumors having an extent of TAP loss that exceeds the stratification cutoff are likely to respond to the MHC-I-dependent immunotherapeutic agent, while tumors having an extent of TAP loss that falls below the stratification cutoff are unlikely to respond in the absence of a dMMR, MSI-H, or TMB-H status. In an exemplary embodiment, the TAP stratification cutoff is selected to differentiate between MHC(+)/pMMR, MHC(+)/MSI-L, and/or MHC(+)/TMB-L tumors that are likely to respond from MHC(+)/pMMR, MHC(+)/MSI-L, and/or MHC(+)/TMB-L tumors that are unlikely to respond.

In another exemplary embodiment, the MHC and TAP stratification cutoffs are set as the median or mean of the extent of loss across a representative population of subjects.

### A. Selection of Mutational Screens

In an embodiment, the stratification cutoffs are used to determine the usefulness of a mutational screen for selecting a patient to receive an MHC-I-directed immunotherapy. In some embodiments, the mutational screening is selected from the group consisting of tumor mutational burden (TMB), mismatch repair (MMR) status, and/or microsatellite instability (MSI) status.

FIG. 1A-1C illustrate some exemplary workflows. In each workflow, the following principles are followed: (a) MHC(+)/TAP(+) tumors are referred for a mutational screen, as the antigenicity of these tumors is likely to be dependent on the mutational load; (b) MHC-I(−) tumors are not referred for a mutational screen regardless of TAP-status, as these tumor are unlikely to be able to present antigens; and (c) MHC-I(+)/TAP(−) tumors do not need to referred for a mutational screen, as they are likely to be antigenic regardless of mutational status.

FIG. 1A illustrates a workflow in which an MHC screen is evaluated first. If the tumor is assessed to be MHC(−), then there is no need for further diagnostic assays, as the tumor is unlikely to be responsive regardless of the TAP or mutational status. If the tumor is MHC(+), a TAP screen status is evaluated. If the tumor is TAP(−), no mutational screen is necessary, as these tumors are likely to be immunogenic regardless of the mutational status. If the tumor is TAP(+), a mutational screen is performed, as the immunogenicity of these tumors is likely to be highly dependent on the mutational status.

FIG. 1B illustrates a workflow in which TAP screen is evaluated first. If the tumor is assessed to be TAP(−), then there is no need for a mutational screen, as tumor immunogenicity is unlikely to depend on the mutational status. Such tumors may be selected to receive the immunotherapy on the basis of MHC status (not illustrated). If the tumor is TAP(+), an MHC screen is evaluated. If the tumor is MHC(−), no mutational screen is necessary, as these tumors are likely to be immunogenic regardless of the mutational status. If the tumor is MHC(+), a mutational screen is performed, as the immunogenicity of these tumors is likely to be highly dependent on the mutational status.

FIG. 1C illustrates a workflow in which TAP and MHC status are evaluated simultaneously (such as when present in a duplex IHC format). In this context, a mutational screen is only performed if the tumor is assessed as MHC(+)/TAP(+), as immunogenicity of these tumors is highly likely to depend on the tumor's mutational status. In all other cases, the tumor is likely to be immunogenic (MHC+/TAP−) or is unlikely to be immunogenic (MHC−/TAP+& MHC−/TAP−) regardless of mutational screen status.

B. Treatment with MHC-I-Dependent Immunotherapies

In an embodiment, the stratification cutoffs are used (optionally in combination with a mutational screen) for selecting a patient to receive an MHC-I-directed immunotherapy. The following principles are generally observed: (a) MHC(−) tumors are not treated with the MHC-I-dependent immunotherapy regardless of TAP-status or mutational screen status, and an alternative treatment course is selected; (b) MHC(+)/TAP(−) tumors receive the MHC-I-dependent immunotherapy regardless of mutational screen status, and an alternate treatment course is selected; and (c) the treatment selected for the MHC-I(+)/TAP (+) patients depends on mutational screen status.

Patients identified as likely to be responsive may then be administered the MHC-I-dependent immunotherapeutic agent according to manufacturer's instructions and recommended treatment course. Patients identified as not likely to be responsive are the referred for alternative treatment courses.

In some embodiments, the assays and methods described herein may be used as a screening test to identify patients eligible for treatment an MHC-I-dependent immunotherapeutic agent. In some embodiments, the assays and methods disclosed herein may be utilized to predict, or assist in predicting, a response to therapy an MHC-I-dependent immunotherapeutic agent; or the results of any assay or method disclosed herein may be used to facilitate treatment with an MHC-I-dependent immunotherapeutic agent. Likewise, the assays and methods described herein may be used to stratify subjects into two or more classes based on their likelihood of responding to treatment an MHC-I-dependent immunotherapeutic agent. For instance, based on the assessment of the expression of the components of the APM, or the constituent elements of the TAP and MHC-I complexes within an obtained biological sample (and/or the assessment of TMB status, MMR status, and/or MSI status), a subject in need of treatment may be stratified into a first class including those subjects likely to respond to an MHC-I-dependent immunotherapeutic agent, or a second class including those subjects likely not to respond to an MHC-I-dependent immunotherapeutic agent.

The present disclosure is also directed to methods of selecting or identifying subjects (e.g., cancer patients) who are appropriate candidates for treatment with a therapy (e.g., with an immunotherapeutic agent) for treatment of cancer. Such individuals include subjects that are predicted to be responsive to the therapy (e.g., with an MHC-I-dependent immunotherapeutic agent) and thus have an increased likelihood of benefiting from administration of the therapy relative to other patients having different characteristic(s) (e.g., non-responsiveness to the therapy). In certain embodiments, an appropriate candidate is one who is reasonably likely to benefit from treatment or at least sufficiently likely to benefit from treatment so as to justify administering the treatment in view of its risks and side effects. The disclosure also encompasses methods of selecting or identifying subjects (e.g., cancer patients) who are not appropriate candidates for treatment with a therapy (e.g., with an MHC-I-dependent immunotherapeutic agent) for treatment of cancer. Such subjects include cancer patients that are predicted to be non-responsive or weakly responsive to the therapy and thus have a decreased likelihood of benefiting from administration of the therapy relative to other patients having different characteristic(s) (e.g., responsiveness to the therapy), or a low or substantially no likelihood of benefiting from such treatment, such that it may be desirable to use a different or additional treatment. In some embodiments, whether a patient is an appropriate candidate for therapy with an MHC-I-dependent immunotherapeutic agent is determined based on an assessment of the expression of the components of the APM or the constituent elements of the TAP and MHC-I complex in a sample derived from the patient, and/or the optionally the assessment of TMB status, MMR status, and/or MSI status.

In some embodiments, the assays and methods disclosed herein may be used in the treatment of cancer (see FIG. 1). For instance, for those subjects whose tumor sample is assessed as TAP(−) and MHC-I(+), a therapeutically effective amount of an MHC-I-dependent immunotherapeutic agent may be administered to treat the subject's cancer. Likewise, for those subjects whose tumor sample is assessed as TAP(+)/MHC-I(+)/TMB-H, a therapeutically effective amount of an MHC-I-dependent immunotherapeutic agent may be administered to treat the subject's cancer. In other embodiments, for those subjects whose tumor sample is assessed as TAP(+)/MHC-I(+)/d-MMR, a therapeutically effective amount of an MHC-I-dependent immunotherapeutic agent may be administered to treat the subject's cancer. In yet other embodiments, for those subjects whose tumor sample is assessed as TAP(+)/MHC-I(+)/MSI-High, a therapeutically effective amount of an MHC-I-dependent immunotherapeutic agent may be administered to treat the subject's cancer.

In some embodiments, the MHC-I-dependent immunotherapeutic agent is a checkpoint inhibitor, a cell-based therapy, or a cancer vaccine therapy. In other embodiments, the MHC-I-dependent immunotherapeutic agent is a checkpoint inhibitor selected from a PD-1 axis-directed therapeutic, a TIM-3-directed therapeutic, and LAG-3-directed therapeutic, and a CTLA-4-directed therapeutic. In some embodiments, the PD-1-axis directed therapeutic is selected from the group consisting of a PD-1-specific antibody, a PD-L1-specific antibody, a PD-1-directed bispecific, a PD-L1-directed bispecific, a PD-1 ligand fragment, a PD-1 ligand fusion protein, and a small molecule inhibitor of PD-1. In some embodiments, a PD-1-axis directed therapy is selected from nivolumab, pembrolizumab, cemiplimab, tislelizumab, spartalizumab, MEDI0680, toripalimab, sintilimab, cetrelimab, and pidilizumab; and the second checkpoint inhibitor is selected from the group consisting of ipilimumab, tremilumab, NLG919, epacadostat, BMS-986205, PF-06840003, navoximod, indoximod, NLG802, LY3381916, MGB453, TSR-022, Sym023, BGBA425, relatlimab, eftilagimod alpha, ieramilimab, REGN3767, and encelimab. In some embodiments, a PD-1-specific antibody selected from nivolumab, pembrolizumab, cemiplimab, tislelizumab, spartalizumab, MEDI0680, toripalimab, sintilimab, cetrelimab, and pidilizumab.

In some embodiments, the cancer is selected from a prostate cancer, a breast cancer, a bladder cancer, a lung cancer, a liver cancer, a cervical cancer, a bile duct cancer, a colon cancer, a rectal cancer, a pancreatic cancer, a uterine cancer, a head and neck cancer, a testicular cancer, a ovarian cancer, a thyroid cancer, a bone cancer, a skin cancer, an adrenal gland cancer, a kidney cancer, a lymphoma, a thymus cancer, a brain cancer, a leukemia, and a cancer of the eye. In some embodiments, the cancer is non-small cell lung cancer (NSCLC). In some embodiments, the cancer is second-line or third line locally advanced or metastatic non-small cell lung cancer. In some embodiments, the cancer is adenocarcinoma. In some embodiments, the cancer is squamous cell carcinoma. In some embodiments, the cancer is non-small cell lung cancer (NSCLC), glioblastoma, neuroblastoma, melanoma, breast carcinoma (e.g., triple-negative breast cancer), gastric cancer, colorectal cancer (CRC), or hepatocellular carcinoma. In some embodiments, the cancer is a primary tumor. In some embodiments, the cancer is a metastatic tumor at a second site derived from any of the above types of cancer.

In yet other embodiments, the present disclosure is directed to methods of treating subjects, e.g., a human patients, having cancer comprising: (a) selecting a subject that is a suitable candidate for treatment with an MHC-I-dependent immunotherapeutic agent; and (b) administering a therapeutically effective amount of the MHC-I-dependent immunotherapeutic agent to the selected subject based on expression of the components of the APM and, in particular, the constituent elements of the TAP complex and MHC-I complex. In some embodiments, the selection of the subject for the treatment with the one or more one or more MHC-I-dependent immunotherapeutic agents comprises (i) obtaining a biological sample from the subject having the cancer; (ii) assessing the expression of the components of the antigen presentation machinery (e.g., the constituent elements of the TAP and MHC-I complexes) in the obtained biological sample; and (iii) selecting the subject candidate for treatment with the one or more one or more immunotherapeutic agents if either: (a) the TAP complex and the MHC-I complex are both intact (TAP(+)/MHC-I(+)), and if the obtained sample is one of TMB-High, MMR-deficient, or MSI-High; or (b) if the TAP complex functionality is lost and the MHC-I complex is functionally intact (TAP(−)/MHC-I(+)).

Exemplary workflows for evaluating the patient's tumor and selecting the patient for treatment are illustrated at FIG. 2A-FIG. 2F.

FIG. 2A illustrates a workflow in which MHC status is used to determine the necessity of subsequent screens. If the tumor sample is MHC(−), no further screening is necessary, as the subject is unlikely to respond to an MHC-I-dependent immunotherapeutic agent and an alternative therapeutic will need to be considered. If the sample is MHC(+), TAP status is used to determine whether the patient will need a mutational screen to select the therapeutic approach. If the sample is TAP(−), the subject may receive an MHC-I-dependent immunotherapeutic agent. If the sample is TAP(+), a mutational screen will be used to select the immunotherapy. In this context, dMMR, MSI-H, and TMB-H samples are all indicated as likely to respond to immunotherapy, whereas pMMR, MSI-L, and TMB-L samples are all indicated as unlikely to respond to immunotherapy and are referred for an alternative therapy.

FIG. 2B illustrates a workflow in which combined MHC/TAP status (such as by using an MHC/TAP duplex AHC assay, a flow cytometry assay co-staining with MHC and TAP in tumor cells, RNAseq assay identifying TAP and MHC expression in tumor cells, etc.) is used to determine the necessity of subsequent screens. If the tumor sample is MHC(−), no further screening is necessary, as the subject is unlikely to respond to an MHC-I-dependent immunotherapeutic agent and an alternative therapeutic will need to be considered. If the sample is MHC(+), TAP status is used to determine whether the patient will need a mutational screen to select the therapeutic approach. If the sample is TAP(−), the subject may receive an MHC-I-dependent immunotherapeutic agent. If the sample is TAP(+), a mutational screen will be used to select the immunotherapy. In this context, dMMR, MSI-H, and TMB-H samples are all indicated as likely to respond to immunotherapy, whereas pMMR, MSI-L, and TMB-L samples are all indicated as unlikely to respond to immunotherapy and are referred for an alternative therapy.

FIG. 2C illustrates an example in which a TAP screen is used to screen patients who would otherwise not be eligible for MHC-I-dependent immunotherapies on the basis of a mutational screen. As in all the other workflows, MHC(−) patients are referred to an alternate therapeutic and MHC(+) patients determined to be dMMR, MSI-H, and/or TMB-H are referred directly to MHC-I-dependent immunotherapeutic agents. Under conventional screening standards, pMMR/MSI-L/TMB-L patients are unlikely to be referred to MHC-I-dependent immunotherapeutic agents. In this workflow, patients having a tumor determined to be MHC(+)/pMMR/MSI-L/TMB-L are screened for TAP expression. Patients having a TAP(−) tumor are selected to receive the MHC-I-dependent immunotherapeutic agent, while TAP(+) patients are referred to an alternate therapy.

FIG. 2D illustrates a workflow in which MHC and TAP screens are used to stratify patients following a mutational screen. The patient's tumor is classified as either a dMMR/MSI-H/TMB-H tumor or a pMMR/MSI-L/TMB-L tumor and then subjected to an MHC screen. For either classification, the patient is referred to an alternative therapy if the tumor is determined to be MHC(−). If the dMMR/MSI-H/TMB-H tumor is determined to be MHC(+), the patient is administered the MHC-I-dependent immunotherapeutic agent.

FIG. 2E illustrates an alternate workflow in which MHC and TAP screens are used to stratify patients following a mutational screen. Patients having a tumor classified as dMMR/MSI-H/TMB-H are referred directly to an MHC screen and the treatment decision is based on the MHC status. Patients having a tumor classified as dMMR/MSI-H/TMB-H are referred directly to an MHC screen and the treatment decision is based on the MHC status. No TAP screen is necessary in this case. For patients having a tumor classified as pMMR/MSI-L/TMB-L, a TAP screen is performed. If the tumor is TAP(+), then an alternate therapy is selected. If the tumor is TAP(−), then an MHC screen is performed. If the TAP(+) tumor is determined to be MHC(−), then an alternate therapy is selected. If the TAP(+) tumor is determined to be MHC(+), then the patient is treated with the MHC-I-dependent immunotherapeutic agent.

FIG. 2F illustrates a workflow in which combined MHC/TAP status (such as by using an MHC/TAP duplex AHC assay, a flow cytometry assay co-staining with MHC and TAP in tumor cells, RNAseq assay identifying TAP and MHC expression in tumor cells, etc.) is used to stratify patients following a mutational screen. For dMMR/MSI-H/TMB-H tumors, patients are administered the MHC-I-dependent immunotherapeutic agent if the tumor is determined to be MHC(+)/TAP(−) or MHC(+)/TAP(+). For pMMR/MSI-L/TMB-L tumors, patients are administered the MHC-I-dependent immunotherapeutic agent if the tumor is determined to be MHC(+)/TAP(−). All other patients are referred to an alternate therapy.

### Anti-Tapi Antibodies

The present disclosure also provides for antibodies immunospecific for human TAP1 useful for, e.g., diagnostic applications (e.g., immunohistochemistry (IHC), immunofluorescence (IF), and immunoblot (e.g., Western blot)).

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three complementarity determining regions which are designated CDR1-H, CDR2-H and CDR3-H and which are supported within framework regions. Each light chain comprises a constant region and a variable region, with the variable region comprising three complementarity determining regions which are designated CDR1-L, CDR2-L and CDR3-L supported by framework regions in an analogous manner to the variable region of the heavy chain.

The complementarity determining regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus, once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

With the above in mind, in one aspect, provided herein is an isolated antibody comprising a heavy chain (HC) immunoglobulin variable domain sequence and a light chain (LC) immunoglobulin variable domain sequence, wherein the heavy chain and light chain immunoglobulin variable domain sequences form an antigen binding site that specifically binds to an epitope contained within the amino acid sequence DGKPLPQYEHRYLHR (SEQ ID NO: 7). This corresponds with amino acid residues 565-579 of SEQ ID NO: 1, the canonical sequence of human TAP1 protein. This particular sequence falls within the ATP-binding cassette (ABC) transporter domain of human TAP1.

An exemplary antibody capable of binding to this sequence of human TAP1 is the monoclonal antibody S14H22L21. The heavy chain variable region (V_{H}) and light chain variable region (V_{L}) of S14H22L21 and their associated CDR and framework (FR) sequences are disclosed at Table 9:

TABLE 9
S14H22L21

| V_{H} | METGLRWLLLVAVLKGVQCQSVKESEGGLFKPTDTLTLTCTVSGFSLSSY | |
|---|---|---|
| | EVIWVRQAPGKGLEWIGNIGWRDTTFYANWAKSRSTFTRDTNLNTVTLK | |
| | MTSLTAADTATYFCARDNGYFGIDYWGPGTLVTVSS (SEQ ID NO: 8) | |
| V_{L} | MDTRAPTQLLGLLLLWLPGARCADIVMTQTPASVEAAVGGTVTIKCQAS | |
| | QSI-SNYLAWYQQKPGQPPKLLIYKA (SEQ ID NO: 9) | |
| | Heavy Chain | Light Chain |
| FR1 | QSVKESEGGLFKPTDTLTLTCTVS | DIVMTQTPASVEAAVGGTVTIKCQAS |
| | (SEQ ID NO: 10) | (SEQ ID NO: 17) |
| CDR1 | GFSLSSYE (SEQ ID NO: 11) | QSISNY (SEQ ID NO: 18) |
| FR2 | VIWVRQAPGKGLEWIGN | LAWYQQKPGQPPKLLIY |
| | (SEQ ID NO: 12) | (SEQ ID NO: 19) |
| CDR2 | IGWRDTT (SEQ ID NO: 13) | KAS (SEQ ID NO: 20) |
| FR3 | FYANWAKSRSTFTRDTNLNTVTLK | TLASGVPSRFSGSGSGTQFTLTISGVQ |
| | MTSLTAADTATYFC | CDDAATYYC (SEQ ID NO: 21) |
| | (SEQ ID NO: 14) | |
| CDR3 | ARDNGYFGIDY (SEQ ID NO: 15) | QQDFNYRNIENV (SEQ ID NO: 22) |
| FR4 | WGPGTLVTVSS (SEQ ID NO: 16) | FGGGTGVVVK (SEQ ID NO: 23) |

In some instances, the anti-human TAP1 antibodies that bind to amino acid residues 565-579 of SEQ ID NO: 1 include at least one, two, three, four, five, or six HVRs selected from (a) CDR1-H comprising the amino acid sequence of SEQ ID NO: 11; (b) CDR2-H comprising the amino acid sequence of SEQ ID NO: 13; (c) CDR3-H comprising the amino acid sequence of SEQ ID NO: 15; (d) CDR1-L comprising the amino acid sequence of SEQ ID NO: 18; (e) CDR2-L comprising the amino acid sequence of SEQ ID NO: 20; and (f) CDR3-L comprising the amino acid sequence of SEQ ID NO: 22. For example, in some instances, the anti-human TAP1 antibodies include (a) an CDR1-H comprising the amino acid sequence of SEQ ID NO: 11; (b) an CDR2-H comprising the amino acid sequence of SEQ ID NO: 13; and (c) an CDR3-H comprising the amino acid sequence of SEQ ID NO: 15. In some instances, the anti-human TAP1 antibodies include (a) an CDR1-L comprising the amino acid sequence of SEQ ID NO: 18; (b) CDR2-L comprising the amino acid sequence of SEQ ID NO: 20; and (c) CDR3-L comprising the amino acid sequence of SEQ ID NO: 22.

In some instances wherein the anti-human TAP1 antibodies bind to amino acid residues 565-579 of SEQ ID NO: 1 and include (a) an CDR1-H comprising the amino acid sequence of SEQ ID NO: 11; (b) an CDR2-H comprising the amino acid sequence of SEQ ID NO: 13; and (c) an CDR3-H comprising the amino acid sequence of SEQ ID NO: 15, the anti-human TAP1 antibodies further include the following heavy chain variable domain framework regions (FRs): (a) FR1-H comprising the amino acid sequence of SEQ ID NO: 10; (b) FR2-H comprising the amino acid sequence of SEQ ID NO: 12; (c) FR3-H comprising the amino acid sequence of SEQ ID NO: 14; or (d) FR4-H comprising the amino acid sequence of SEQ ID NO: 16. In some instances wherein the anti-human TAPI antibodies bind to amino acid residues 565-579 of SEQ ID NO: 1 and include (a) an CDR1-H comprising the amino acid sequence of SEQ ID NO: 11; (b) an CDR2-H comprising the amino acid sequence of SEQ ID NO: 13; and (c) an CDR3-H comprising the amino acid sequence of SEQ ID NO: 15, the anti-human TAP1 antibodies further include the following heavy chain variable domain framework regions (FRs): (a) FR1-H comprising the amino acid sequence of SEQ ID NO: 10; (b) FR2-H comprising the amino acid sequence of SEQ ID NO: 12; (c) FR3-H comprising the amino acid sequence of SEQ ID NO: 14; and (d) FR4-H comprising the amino acid sequence of SEQ ID NO: 16.

In some instances wherein the anti-human TAP1 antibodies bind to amino acid residues 565-579 of SEQ ID NO: 1, the antibodies include (a) an CDR1-H comprising the amino acid sequence of SEQ ID NO: 11; (b) an CDR2-H comprising the amino acid sequence of SEQ ID NO: 13; (c) an CDR3-H comprising the amino acid sequence of SEQ ID NO: 15; (d) an CDR1-L comprising the amino acid sequence of SEQ ID NO: 18; (e) an CDR2-L comprising the amino acid sequence of SEQ ID NO: 20; and (f) an CDR3-L comprising the amino acid sequence of SEQ ID NO: 22. In some instances, these anti-human TAP1 antibodies include the following FRs: (a) FR1-H comprising the amino acid sequence of SEQ ID NO: 10; (b) FR2-H comprising the amino acid sequence of SEQ ID NO: 12; (c) FR3-H comprising the amino acid sequence of SEQ ID NO: 14; and (d) FR4-H comprising the amino acid sequence of SEQ ID NO: 16 and may additionally or alternatively include (e) FR1-L comprising the amino acid sequence of SEQ ID NO: 17; (f) FR2-L comprising the amino acid sequence of SEQ ID NO: 19; (g) FR3-L comprising the amino acid sequence of SEQ ID NO: 21; and (h) FR4-L comprising the amino acid sequence of SEQ ID NO: 23.

In some instances, the anti-human TAP1 antibodies that bind to amino acid residues 565-579 of SEQ ID NO: 1 may also include a heavy chain variable domain (V_{H}) sequence having at least 80% (e.g., at least 81%, 82%, 83%, 84%, 85%, 86%, 87%, 88%, or 89%), at least 90% (e.g., at least 91%, 92%, 93%, or 94%), or at least 95% (e.g., at least 96%, 97%, 98%, or 99%) sequence identity to, or the sequence of, the amino acid sequence of SEQ ID NO: 16. In certain embodiments, a VH sequence having at least 80%, 81%, 82%, 83%, 84%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence (SEQ ID NO: 8), but an anti-human TAP1 antibody including that sequence retains the ability to bind to SEQ ID NO: 1. In certain embodiments, a total of 1 to 10 amino acids (e.g., 1, 2, 3, 4, 5, 6, 7, 8, 9, or 10 amino acids) have been substituted, inserted, and/or deleted in SEQ ID NO: 8. In certain embodiments, substitutions, insertions, or deletions occur in regions outside the HVRs (i.e., in the FRs). Optionally, the anti-human TAP1 antibodies include the VH sequence in SEQ ID NO: 16, including post-translational modifications of that sequence. In a particular embodiment, the V_{H} comprises one, two, or three HVRs selected from: (a) CDR1-H comprising the amino acid sequence of SEQ ID NO: 11, (b) CDR2-H comprising the amino acid sequence of SEQ ID NO: 13, and (c) CDR3-H comprising the amino acid sequence of SEQ ID NO: 15.

In some instances, the anti-human TAP1 antibodies that bind to amino acid residues 565-579 of SEQ ID NO: 1 may also include a light chain variable domain (V_{L}) having at least 80% (e.g., at least 81%, 82%, 83%, 84%, 85%, 86%, 87%, 88%, or 89%), at least 90% (e.g., at least 91%, 92%, 93%, or 94%), or at least 95% (e.g., at least 96%, 97%, 98%, or 99%) sequence identity to, or the sequence of, the amino acid sequence of SEQ ID NO: 9. In certain embodiments, a VL sequence having at least 80%, 81%, 82%, 83%, 84%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence (SEQ ID NO: 9), but an anti-human TAP1 antibody including that sequence retains the ability to bind to SEQ ID NO: 1. In certain embodiments, a total of 1 to 10 amino acids (e.g., 1, 2, 3, 4, 5, 6, 7, 8, 9, or 10 amino acids) have been substituted, inserted, and/or deleted in SEQ ID NO: 9. In certain embodiments, the substitutions, insertions, or deletions occur in regions outside the HVRs (i.e., in the FRs). Optionally, the anti-human TAP1 antibody comprises the V_{L} sequence in SEQ ID NO: 9, including post-translational modifications of that sequence. In a particular embodiment, the V_{L} comprises one, two or three HVRs selected from (a) CDR1-L comprising the amino acid sequence of SEQ ID NO: 18; (b) CDR2-L comprising the amino acid sequence of SEQ ID NO: 20; and (c) CDR3-L comprising the amino acid sequence of SEQ ID NO: 22.

In some instances, the anti-human TAP1 antibodies that bind to amino acid residues 565-579 of SEQ ID NO: 1 include both V_{H} and V_{L} sequences having at least 80% (e.g., at least 81%, 82%, 83%, 84%, 85%, 86%, 87%, 88%, or 89%), at least 90% (e.g., at least 91%, 92%, 93%, or 94%), or at least 95% (e.g., at least 96%, 97%, 98%, or 99%) sequence identity to, or the sequences of, the amino acid sequences of SEQ ID NOs: 8 and 9, respectively, and may or may not include post-translational modifications of those sequences.

In other instances, the present disclosure provides antibodies that specifically bind SEQ ID NO: 1, wherein the antibodies include (a) an CDR1-H comprising the amino acid sequence of SEQ ID NO: 11; (b) an CDR2-H comprising the amino acid sequence of SEQ ID NO: 13; (c) an CDR3-H comprising the amino acid sequence of SEQ ID NO: 15; (d) an CDR1-L comprising the amino acid sequence of SEQ ID NO: 18; (e) an CDR2-L comprising the amino acid sequence of SEQ ID NO: 20; and (f) an CDR3-L comprising the amino acid sequence of SEQ ID NO: 22. In some instances, these anti-human TAP1 antibodies include the following FRs: (a) FR1-H comprising the amino acid sequence of SEQ ID NO: 10; (b) FR2-H comprising the amino acid sequence of SEQ ID NO: 12; (c) FR3-H comprising the amino acid sequence of SEQ ID NO: 14; and (d) FR4-H comprising the amino acid sequence of SEQ ID NO: 16 and may additionally or alternatively include (e) FR1-L comprising the amino acid sequence of SEQ ID NO: 17; (f) FR2-L comprising the amino acid sequence of SEQ ID NO: 19; (g) FR3-L comprising the amino acid sequence of SEQ ID NO: 21; and (h) FR4-L comprising the amino acid sequence of SEQ ID NO: 23. In some embodiments, for example, the anti-human TAP1 antibodies include both a V_{H} and a V_{L} sequence including the sequences of the amino acid sequences of SEQ ID NOs: 8 and 9, respectively, and may or may not include post-translational modifications.

### Kits

The present disclosure also provides for kits including antibodies and detection reagents suitable for staining a sample in a multiplex or simplex immunoenzymatic assay. In some embodiments, the kit comprises (i) a pair of primary antibodies, and (ii) optionally detection agents for performing an immunoenzymatic assay, wherein the pair of primary antibodies comprises: (a) at least one of an anti-TAP1 antibody and an anti-TAP2 antibody; and (b) one of an anti-HLA-A antibody, an anti-HLA-B antibody, and an anti-HLA-C antibody. In some embodiments, the kit comprises both an anti-TAP1 antibody and an anti-TAP2 antibody. In some embodiments, the anti-TAP1 antibody and/or the anti-TAP2 antibody is a mouse or a rabbit monoclonal antibody. In some embodiments, the anti-TAP1 antibody is an anti-human TAP1 antibody as described in the section entitled “ANTI-TAP1 ANTIBODIES.” In some embodiments, the anti-HLA antibody is a rabbit monoclonal antibody or a mouse monoclonal antibody. In some embodiments, the kit further comprises a secondary antibody specific to the anti-TAP1 or anti-TAP2 antibody, wherein the secondary antibody specific to the anti-TAP1 or anti-TAP2 antibody is conjugated to a peroxidase enzyme or an alkaline phosphatase enzyme. In some embodiments, the kit further comprises a secondary antibody specific to the anti-HLA antibody, wherein the secondary antibody specific to the anti-HLA antibody is conjugated to a peroxidase enzyme or an alkaline phosphatase enzyme. In some embodiments, the kit further comprises a first chromogenic agent and a second chromogenic agent. In some embodiments, at least one of the first or second chromogenic agents is 3,3′-diaminobenzidine (DAB). In some embodiments, at least one of the first or second chromogenic agents is Fast Red. In some embodiments, a first chromogenic agent is one of DAB or Fast Red; and the second chromogenic agent is the other of DAB or Fast Red.

### EXAMPLES

### Example 1—TAP1 Antibody Generation

anti-TAP1 rabbit monoclonal primary antibodies were directed against the sequence DGKPLPQYEHRYLHR (SEQ ID NO: 23), which represents amino acids 625-640 of human TAP1. The peptide was synthesized and covalently conjugated to keyhole limpet haemocyanin (KLH) carrier protein. Rabbits were immunized with KLH-conjugated peptide with complete Freund's adjuvant followed by a series of booster injections of the same immunogen in incomplete Freund's adjuvant. The rabbit that produced the best positive polyclonal antibody by immunohistochemistry (IHC) was chosen for monoclonal development. IHC staining was performed on a BenchMark automated slide stainer at Roche Diagnostics in Tucson, AZ. The staining procedure included deparaffinization, pretreatment using standard Cell Conditioning 1, incubation with antibody (TAP1: 0.925 μg/mL) at 36° C. for 16 min. Following the chromogenic detection, all slides were counterstained with Hematoxylin II and Bluing Reagent (Ventana) for 4 min each and coverslips were applied. TAP1 was detected by immunohistochemistry (IHC) using OptiView Universal DAB detection kit (Ventana).

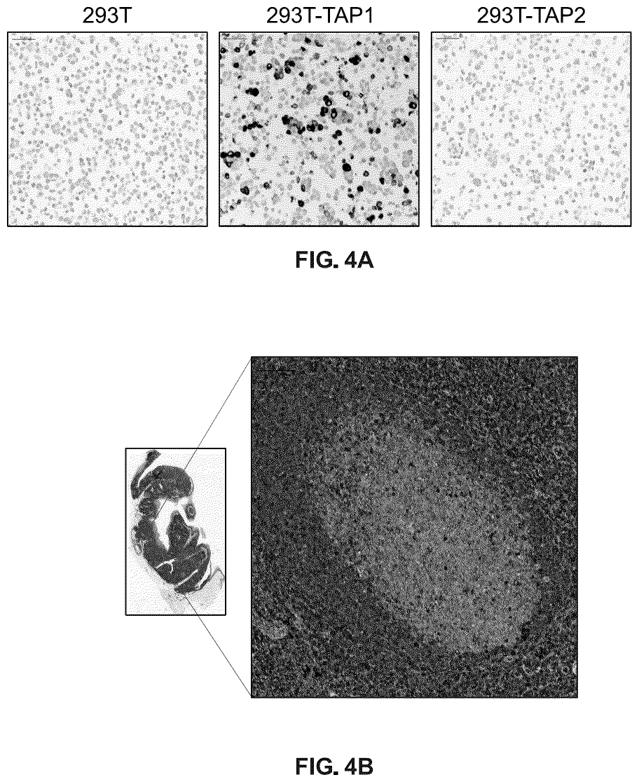

Antibody expressing cells were isolated and screened by a standard enzyme-linked immunoabsorbent assay (ELISA) for reactivity to the immunogenic peptide described above, and by IHC assays on control cell blocks made from HEK 293T cells overexpressing recombinant TAP1. Once IHC positive antibody producing cells were identified, the cDNAs coding for the antibody heavy chain and light chain were isolated and cloned using standard recombinant techniques. Monoclonal antibodies were produced by co-transfecting the cloned heavy and light chain cDNAs. The antibody functionality was confirmed by IHC staining of control cell blocks consisting of HEK293T cells and HEK293T cells expressing recombinant TAP2 (negative controls), and HEK293T cells expressing recombinant TAP1 and tonsil tissue (positive controls) (see FIGS. 4A and 4B). Rabbit anti-human TAP1 monoclonal antibodies with the best specificity, including S14H22L21, were selected and purified through a Protein A column. The heavy chain variable region (V_{H}) and light chain variable region (V_{L}) sequences for S14H22L21 are disclosed above in Table 9, along with their associated framework regions (FR) and complementarity determining regions (CDR) (determined according to IMGT numbering system).

### Example 2—Characterization and Specificity of TAP1 Antibody

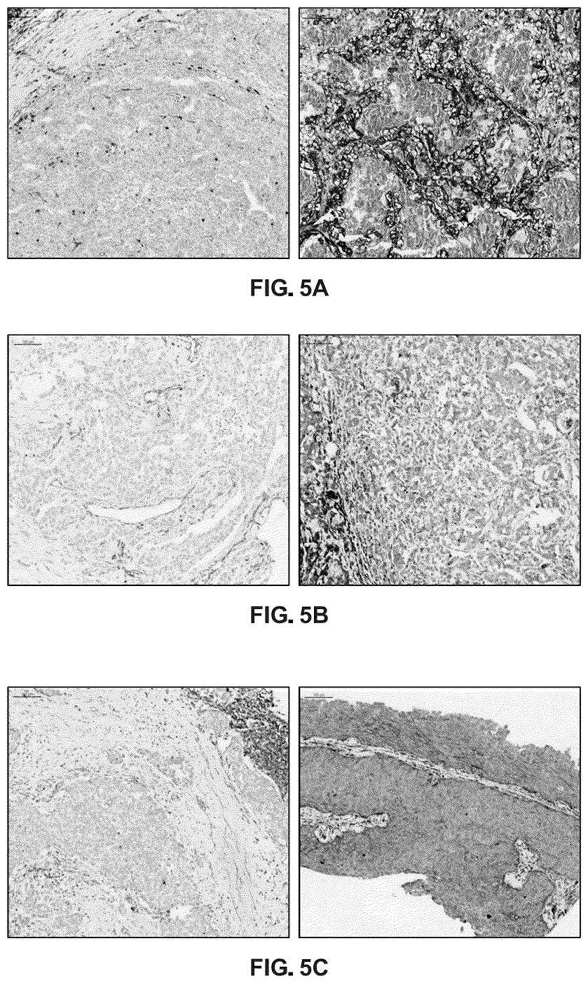

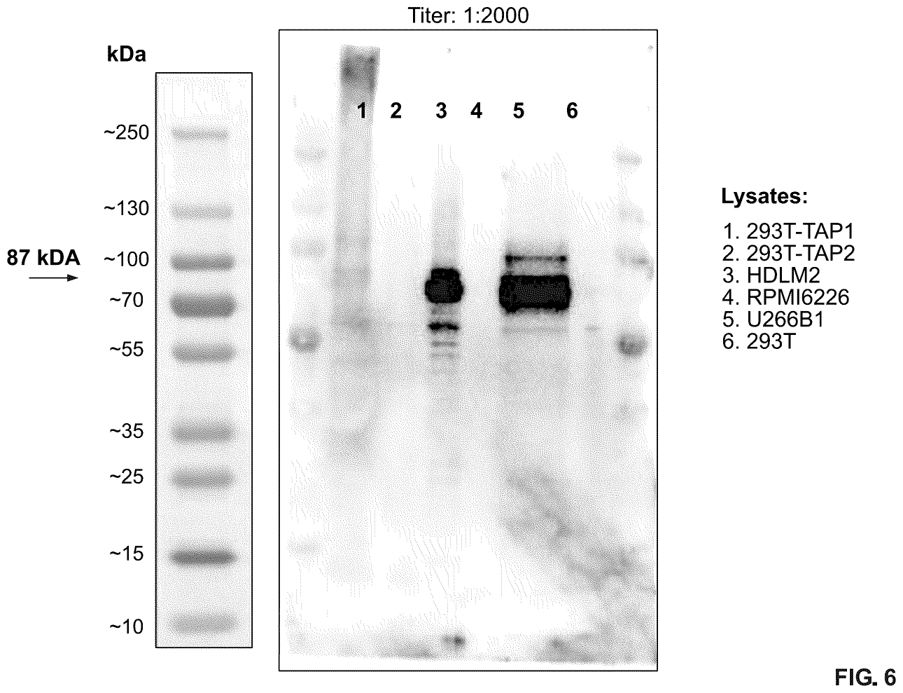

Rabbit monoclonal antibody S14H22L21 was applied onto formalin fixed paraffin embedded tissue samples to assess the staining patterns of the antibody by IHC as described in Example 1. Tissue samples included the positive and negative controls listed in Example 1, 8 ccRCC tissues, and a tour of tumor array (see FIGS. 4A-4B and 5A-5C). Western blot analysis was carried out to assess the specificity of S14H22L21. Cell lysates from HEK293T cells, HEK293T cells expressing TAP1, and HEK293T cells expressing TAP2 were analyzed alongside lysates from a negative control cell line (RPMI6226) and two positive control cell lines (HDLM2 and U266B1). S14H22L21 bound to an 87 kDa protein in the positive control cell lines, and to lysates from 293T cell lines expressing TAP1, but not to negative control cell lines, or lysates expressing TAP2 (FIG. 6). The data demonstrates the specificity of the TAP1 antibody. The antibody characterization showed strong specificity of the TAP 1 antibody because it bound to a protein that was the correct size of TAP1, stained cell blocks from HEK293T cells expressing TAP1, and failed to stain HEK293T cell blocks expressing TAP2. This staining also revealed several cases with partial or complete loss of TAP1, including ccRCC, invasive ductal carcinoma of the breast, and transitional cell carcinoma of the bladder (FIGS. 5A-5C).

### Example 3—TAP1 and MHC-I Expression in CCRCC

Tissue microarrays were purchased from Pantomics, Inc. Clear cell renal cell carcinoma cases were part of the Kidney cancer tissue array (KIC1021), which contained 95 total cancer cases and 5 normal/benign cases. All tissues were from surgical resection. They were fixed in 10% neutral buffered formalin for 24 hours and processed using identical standard operating procedures. Sections were picked up onto Superfrost Plus or APES coated Superfrost slides. They were used within 6 months of the purchase date. TAP1 and HLA-A were detected by immunohistochemistry (IHC) using mouse anti-TAP1 monoclonal antibody (clone S14H22L21) and mouse anti HLA-A monoclonal antibody (clone EP1395Y). HLA-A was purchased from Abcam. All assays were performed on a BenchMark Ultra automated slide stainer at Roche Diagnostics in Tucson, AZ.

Proteins on sections from TMAs were detected using OptiView DAB IHC detection kit (Ventana). The staining procedure included deparaffinization, pretreatment using standard Cell Conditioning 1, incubation with antibody (TAP1: 0.925 μg/mL, HLA-A 0.13 μg/mL) at 36° C. for 16 min. Following the chromogenic detection, all slides were counterstained with Hematoxylin II and Bluing Reagent (Ventana) for 4 min each and coverslips were applied. Slides were scored for TAP1 and HLA-A as either having intact expression in tumor cells or having complete or heterogeneous loss of expression in tumor cells, relative to the intensity present in infiltrating immune cells.

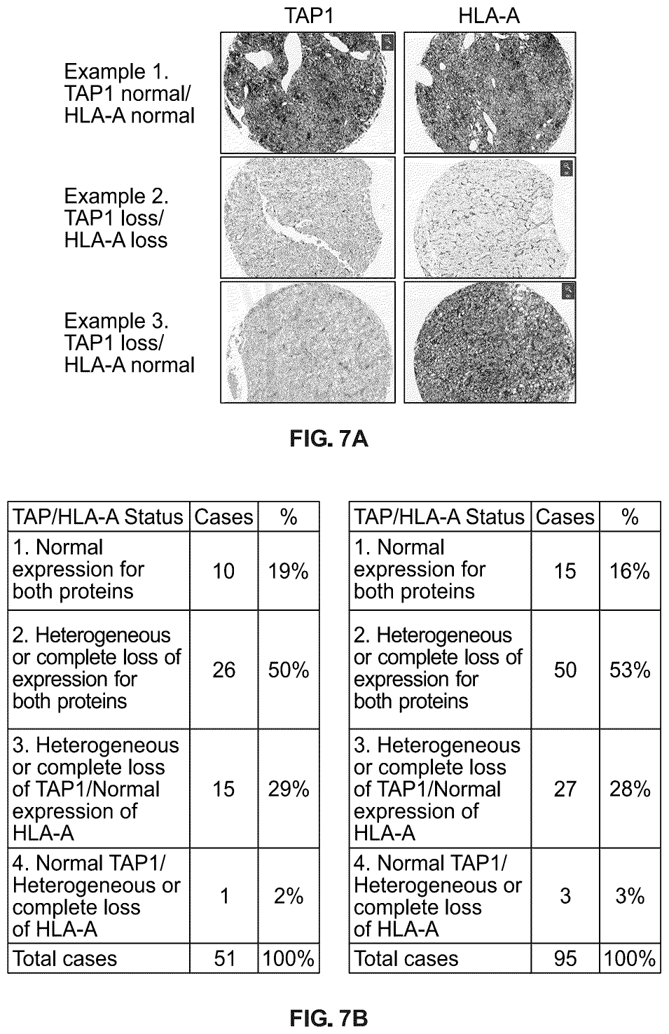

FIGS. 7A and 7B show that only around 20% of ccRCC patients had intact expression for TAP and MHC-I, while 30% had heterogeneous or complete loss of TAP expression with intact MHC-I. Surprisingly, 50% of ccRCC patients had heterogeneous or complete loss of expression of both TAP and MHC-I. These results support that an alternate pathway of neoantigen presentation may exist in ccRCC tumor cells.

### Example 4—TAP1 and MHC-I Expression in Lung Cancer

Lung cancer cases were part of the Lung cancer tissue array LUC1021 from Pantomics which contained 97 total cancer cases and 5 normal/benign cases. All tissues were from surgical resection. They were fixed in 10% neutral buffered formalin for 24 hours and processed using identical standard operating procedures. Sections were picked up onto Superfrost Plus or APES coated Superfrost slides. They were used within 6 months of the purchase date. TAP1 and HLA-A were detected by immunohistochemistry (IHC) using mouse anti-TAP1 monoclonal antibody (clone S14H22L21) and mouse anti HLA-A monoclonal antibody (clone EP1395Y). HLA-A was purchased from Abcam. All assays were performed on a BenchMark Ultra automated slide stainer at Roche Diagnostics in Tucson, AZ.

Proteins on sections from TMAs were detected using OptiView DAB IHC detection kit (Ventana). The staining procedure included deparaffinization, pretreatment using standard Cell Conditioning 1, incubation with antibody (TAP1: 0.925 μg/mL, HLA-A 0.13 μg/mL) at 36° C. for 16 min. Following the chromogenic detection, all slides were counterstained with Hematoxylin II and Bluing Reagent (Ventana) for 4 min each and coverslips were applied. Slides were scored for TAP1 and HLA-A as either having intact expression in tumor cells or having complete or heterogeneous loss of expression in tumor cells, relative to the intensity present in infiltrating immune cells.

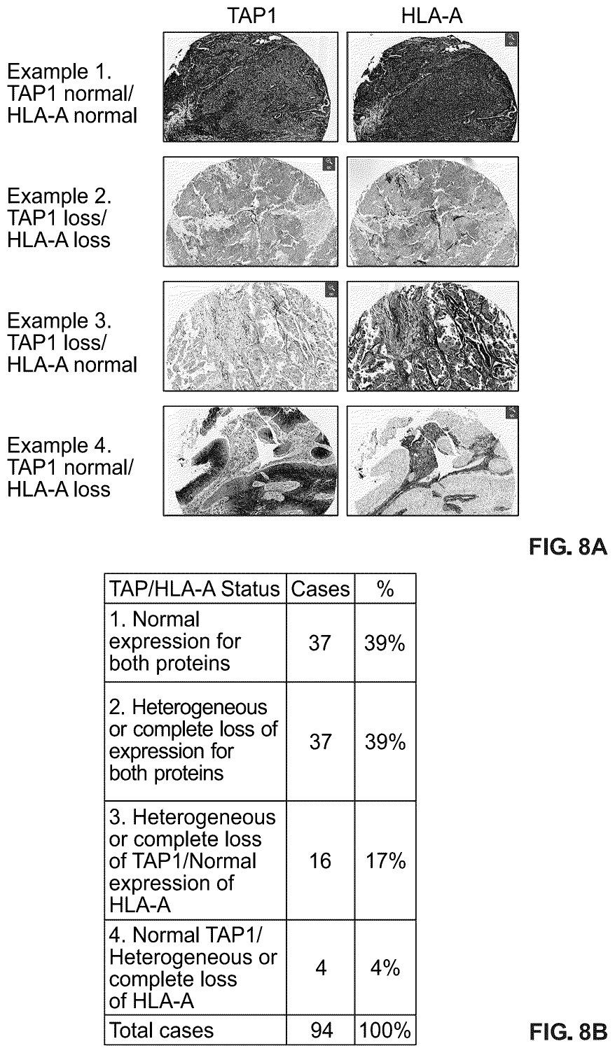

FIGS. 8A and 8B demonstrate that in lung cancer, where TMB is more reliably associated with responsiveness to MHC-I-dependent immunotherapeutic agent, around 40% of patients had intact expression for TAP and MHC-I. However, even in lung cancer, 17% of patients had loss of TAP with intact MHC-I. These results support that even in a disease area where tumor mutational burden reliably predicts response to anti-PD1/PD-L1 therapy, an alternate pathway of neoantigen presentation could exist, and an assay to detect this mode of neoantigen presentation may identify patients who could respond to anti-PD1/PD-L1 therapy.

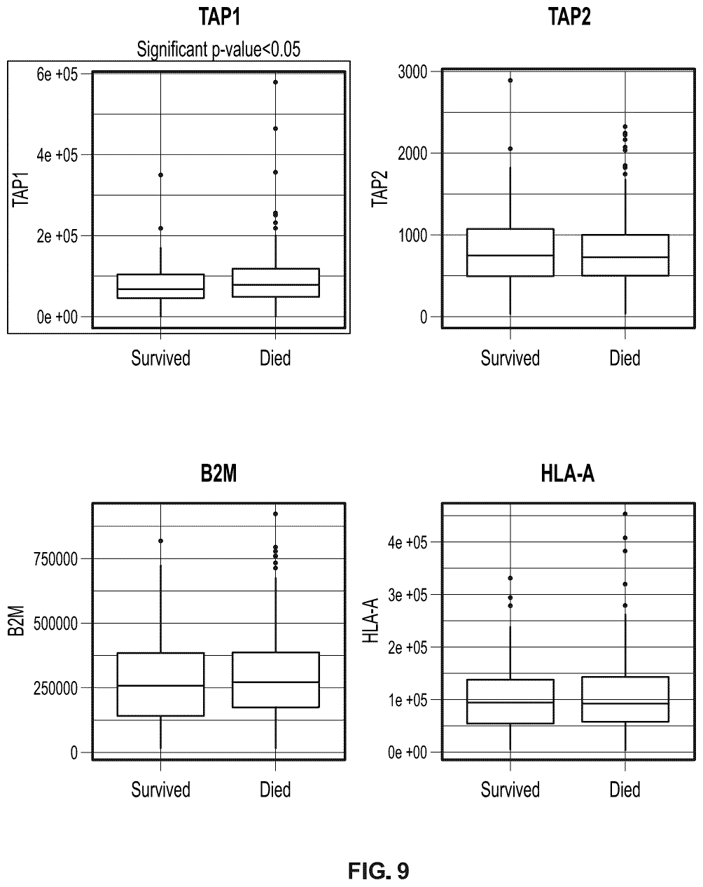

FIG. 9 illustrates a RAAD dataset of RNAseq data from patients who died or survived on immunotherapy in different disease states. FIG. 9 further shows bulk RNA expression of antigen presentation components TAP1, TAP2, B2M, and HLA-A in ccRCC patients who survived or died on immunotherapy. Lower expression of TAP1 alone was significantly associated with survival on immunotherapy.

### Example 5—Assessment of Tap1/2 RNA Expression in CCRCC

To investigate if expression of APM genes predict CIT response we used the 2019-curated Enhanced Data and Insights Sharing (EDIS) CIT datamart datasets of response to CIT after 1 year treatment with Tecentric and gene expression (RNA). The data available spans multiple trials and has been normalized to account for batch and sequencing depth effects. These trials have clinical data linked to information on the patient's genomic profile such as RNAseq and PDL1 IHC. This was the RAAD2.0 data challenge dataset.

Whole RNA-seq data was subset to pathway proteins of interest, the following indications were available in the dataset: “Advanced or metastatic NSCLC, “Advanced or Metastatic Urothelial Bladder Cancer,” “Stage IV Non-Squamous NSCLC,” Untreated Extensive-Stage Small Cell Lung Cancer”, “Untreated Advanced Renal Cell Carcinoma”.

Response endpoint in this dataset is defined by 1-year survival after treatment with Tecentriq. The total data comprised of 27% Surviving patients and 73% Dead (need better word). Further breakdown on table below.

The working hypothesis was HO=difference in mean of gene distribution is equal to 0, If the results show a p-value <0.01 it supports the alternative hypothesis H1=“true difference in means is not equal to 0;” essentially it states there is a statistical difference between the two means. Due to the non-normality concerns this test was performed in two ways to ensure our results are not being biased due to assumption violations. Welch Two Sample t-test (native in R) was performed in non-transformed and log transformed data and the nonparametric Wilcoxon rank sum test (package link) with continuity correction between each the RNA-seq normalized count of all patients data grouped by indication and the survived or dead Boolean label per gene, significance in all 3 of these statistics were shared as proposed targets for investigation in site. The proposed method was applied to housekeeping genes for sanity (GAPDH and ACTB).

Although most of the genes in most indications had marginal to no signal, TAP1 in the RCC dataset passed all 3 statistics tests:

Welch Two Sample t-Test:
##data: TAP1 by Survived ##t=−2.8767, df=356.42, p-value=0.00426
##alternative hypothesis: true difference in means is not equal to 0
##95 percent confidence interval:
##−27559.34-5178.51
##sample estimates:
##mean in group Survived mean in group Dead
##75881.51 92250.43
   Welch Two Sample t-Test Log
##data: log (TAP1) by Survived
##t=−2.7889, df=218.83, p-value=0.005755
   Wilcoxon Rank Sum Test with Continuity Correction
##data: TAP1 by Survived
##W=19497, p-value=0.02621
##alternative hypothesis: true location shift is not equal to 0

The dataset had minimum missing values on RNA-seq calls:

| | Percent | | | |
|---|---|---|---|---|
| Cancer | of total | Count of patients | % survived | % died |
| ##Advanced | 0.37 | 2110 | 0.19 | 0.81 |
| or | | | | |
| metastatic | | | | |
| NSCLC - | | | | |
| ## | 0.26 | 1509 | 0.15 | 0.85 |
| Advanced | | | | |
| or | | | | |
| Metastatic | | | | |
| Urothelial | | | | |
| Bladder | | | | |
| Cancer - | | | | |
| ## Stage IV | 0.24 | 1381 | 0.44 | 0.56 |
| Non- | | | | |
| Squamous | | | | |
| NSCLC - | | | | |
| ##Untreated | 0.08 | 463 | 0.3 | 0.7 |
| Advanced | | | | |
| Renal Cell | | | | |
| Carcinoma - | | | | |
| ## | 0.05 | 277 | 0.67 | 0.33 |
| Untreated | | | | |
| Extensive- | | | | |
| Stage Small | | | | |
| Cell Lung | | | | |
| Cancer - | | | | |

### Genes

### Gene ID Ensemble P-Value T-Test Between CIT Response/Survival and Death

GAPDH ENSG00000111640 p-value=0.7734
ACTB ENSG00000075624 p-value=0.3547
TAP2 ENSG00000204267 p-value=0.6331
TAP1 ENSG00000168394 p-value=0.00426
TAPBP ENSG00000231925 p-value=0.9524
TABPL ENSG00000139192 p-value=0.05824
CALR ENSG00000179218 p-value=0.03727
ERAP1 ENSG00000164307 p-value=0.638
ERAP2 ENSG00000164308 p-value=0.5453
HLA-A ENSG00000206503 p-value=0.6818
HLA-B ENSG00000234745 p-value=0.07164
HLA-C ENSG00000204525 p-value=0.006255
B2M ENSG00000166710 p-value=0.08874
PDIA3 ENSG00000167004 p-value=0.4225

### Example 6 (Prophetic)

Our study design uses a retrospective cohort. We will search the electronic medical record (EMR) to identify patients with ccRCC treated with PD-1 blockade to assess response rate, depth of response (CR, PR, SD), duration of disease control and progression-free and overall survival. We will identify patients by query of medical records and therapy databases. Screened cases will be included in the study if the patients meet the inclusion criteria. The EMR of patients who meet eligibility criteria will be reviewed for collection of demographics, clinical, histopathological and treatment data. Prior to analysis, data will be entered in an encrypted database using sequentially generated patient identifiers to preserve the anonymity of the patients.

We will also receive and review data from patients with ccRCC treated with a PD-1/PD-L1 immune checkpoint molecule in the adjuvant setting.

Archived/residual formalin-fixed paraffin-embedded (FFPE) tumor tissue blocks of the study participants (from the standard treatment setting and from the adjuvant treatment setting) will be retrieved and histologic sections will be obtained (1 H&E stained section from each block, 10 sections from each block, 4 microns thick, for IHC studies; 5 sections, 10 microns thick for genomic studies). TAP1 and MHC-I staining will be assessed by a pathologist relative to staining of infiltrating normal cells (e.g., immune, blood vessels) which normally express robust levels of TAP and MHC-I. Any slides with normal cells staining negative for TAP or MHC-I will be considered compromised by unfavorable preanalytic variables and will be excluded. Genomic DNA will be isolated from five 10-micron slides and sent to a service provider for sequencing library preparation and exome sequencing. Sequencing data analysis will be performed by Roche or the Institution, and TMB will be calculated using standard methods (Zehir, et al 2017, Xu, et al 2019). Exploratory studies of the immune microenvironment will be conducted on the remaining 4-micron slides, pending results of TAP and MHC-I staining and correlation with response to therapy. Statistical analysis will be conducted to explore the correlation between the readouts and tumor response/duration of disease control. Survival analysis may be done using the method described by Kaplan and Meier. Overall survival will be defined as the time from diagnosis to death due to any cause or the date of last follow up. Progression-free survival will be defined as the time from the initial treatment to disease progression from the date of last follow-up. Differences between survival curves will be tested for statistical significance using the two-sided log-rank test.

### ADDITIONAL EMBODIMENTS

Additional Embodiment 1 A method of determining whether a tumor sample derived from a subject should be referred for a tumor mutational screen, the method comprising evaluating, in cells of the tumor sample, (i) an expression of the constituent elements of a transporter associated with antigen processing (TAP) complex, and (ii) an expression of the constituent elements of the major histocompatibility complex class I (MHC); wherein the tumor sample is referred for the tumor mutational screen when the tumor sample is evaluated to be MHC-I(+) and TAP(+).
Additional Embodiment 2 The method of additional embodiment 1, wherein the tumor mutational screen is selected from the group consisting of mismatch repair (MMR) screening, microsatellite instability (MSI) screening, and tumor mutational burden screening (TMB).
Additional Embodiment 3 The method of additional embodiment 2, wherein if the tumor sample is evaluated to be MMR deficient (dMMR), MSI high, and/or TMB high, the tumor sample is likely to respond to an MCH-I dependent immunotherapeutic agent.
Additional Embodiment 4 The method of additional embodiment 2, wherein if the tumor sample is evaluated to be MMR proficient (pMMR), MSI low, and/or TMB low, the tumor sample is unlikely to respond to an MCH-I dependent immunotherapeutic agent.
Additional Embodiment 5 The method of any one of additional embodiments 3 and 4, wherein the MCH-I dependent immunotherapeutic agent is selected from the group consisting of a checkpoint inhibitor, a cell-based therapy, and a cancer vaccine therapy.
Additional Embodiment 6 A method of stratifying a tumor sample previously determined to be pMMR, MSS or MSI-L, and/or TMB-L by a tumor mutational screen, the method comprising evaluating, in cells of the tumor sample, (i) an expression of the constituent elements of a transporter associated with antigen processing (TAP) complex, and (ii) an expression of the constituent elements of a major histocompatibility complex class I (MHC), wherein:
   a. if the tumor sample is evaluated to be TAP(−)/MHC(+), the tumor sample is likely to respond to an MHC-I-dependent immunotherapeutic agent; and
   b. if the tumor sample is evaluated to be TAP(+)/MHC(+), TAP(+)/MHC (−), or TAP(−)/MHC(−), the tumor sample is unlikely to respond to the MHC-I-dependent immunotherapeutic agent.
Additional Embodiment 7 The method of additional embodiment 6, wherein the MCH-I dependent immunotherapeutic agent is selected from the group consisting of a checkpoint inhibitor, a cell-based therapy, and a cancer vaccine therapy.
Additional Embodiment 8 A method of stratifying a tumor sample previously determined to be pMMR, MSS or MSI-L, and/or TMB-L by a tumor mutational screen and also previously determined to be MHC(+), the method comprising evaluating, in cells of the tumor sample, an expression of the constituent elements of a transporter associated with antigen processing (TAP) complex, wherein:
   a. if the tumor sample is evaluated to be TAP(−), the tumor sample is likely to respond to an MHC-I-dependent immunotherapeutic agent; and
   b. if the tumor sample is evaluated to be TAP(+), tumor sample is unlikely to respond to the MHC-I-dependent immunotherapeutic agent.
Additional Embodiment 9 The method of additional embodiment 8, wherein the MCH-I dependent immunotherapeutic agent is selected from the group consisting of a checkpoint inhibitor, a cell-based therapy, and a cancer vaccine therapy.
Additional Embodiment 10 A method of stratifying a tumor sample previously determined to be pMMR, MSS or MSI-L, and/or TMB-L by a tumor mutational screen and also previously determined to be TAP(−), the method comprising evaluating, in cells of the tumor sample, an expression of the constituent elements of a major histocompatibility complex class I (MHC), wherein:
   a. if the tumor sample is evaluated to be MHC(+), the tumor sample is likely to respond to an MHC-I-dependent immunotherapeutic agent; and
   b. if the tumor sample is evaluated to be MHC(−), tumor sample is unlikely to respond to the MHC-I-dependent immunotherapeutic agent.
Additional Embodiment 11 The method of additional embodiment 10, wherein the MCH-I dependent immunotherapeutic agent is selected from the group consisting of a checkpoint inhibitor, a cell-based therapy, and a cancer vaccine therapy.
Additional Embodiment 12 A method of stratifying a tumor sample previously determined to be dMMR, MSI-H, and/or TMB-H by a tumor mutational screen, the method comprising evaluating, in cells of the tumor sample, (i) an expression of the constituent elements of a transporter associated with antigen processing (TAP) complex, and (ii) an expression of the constituent elements of a major histocompatibility complex class I (MHC), wherein:
   a. if the tumor sample is evaluated to be TAP(−)/MHC(+) or TAP(+)/MHC (+), the tumor sample is likely to respond to an MHC-I-dependent immunotherapeutic agent; and
   b. if the tumor sample is evaluated to be TAP(+)/MHC(−), or TAP(−)/MHC (−), the tumor sample is unlikely to respond to the MHC-I-dependent immunotherapeutic agent.
Additional Embodiment 13 The method of additional embodiment 12, wherein the MCH-I dependent immunotherapeutic agent is selected from the group consisting of a checkpoint inhibitor, a cell-based therapy, and a cancer vaccine therapy.
Additional Embodiment 14 A method of selecting a subject having a tumor to receive an MHC-I-dependent immunotherapeutic agent, the method comprising (i) evaluating, in cells of the tumor, an expression of the constituent elements of a transporter associated with antigen processing (TAP) complex, (ii) evaluating, in cells of the tumor, an expression of the constituent elements of a major histocompatibility complex class I (MHC), and (iii) optionally evaluating a tumor mutational status; wherein the subject is selected to receive the MHC-I-dependent immunotherapeutic agent if: (i) the tumor is evaluated to be MHC(+) and TAP(−) regardless of tumor mutational status, or (ii) if the tumor is evaluated to be MHC(+), TAP(+), and one or more of dMMR, MSI-H, or TMB-H.
Additional Embodiment 15 The method of additional embodiment 14, wherein the optional evaluation of the tumor mutational status comprises performing a tumor mutational screen selected from the group consisting of mismatch repair (MMR) screening, microsatellite instability (MSI) screening, and tumor mutational burden screening (TMB).
Additional Embodiment 16 The method of any one of additional embodiments 14 and 15, wherein the MHC-I-dependent immunotherapeutic agent is selected from the group consisting of a checkpoint inhibitor, a cell-based therapy, and a cancer vaccine therapy.
Additional Embodiment 17 A method of selecting a subject having a tumor to receive an MHC-I-dependent immunotherapeutic agent, wherein the tumor has been previously classified to be pMMR, MSS or MSI-L, and/or TMB-L by a tumor mutational screen, the method comprising evaluating, in cells of the tumor sample, (i) an expression of the constituent elements of a transporter associated with antigen processing complex, and (ii) an expression of the constituent elements of a major histocompatibility complex class I; wherein the subject is selected to receive the MHC-I-dependent immunotherapeutic agent if the tumor is evaluated to be TAP(−)/MHC(+).
Additional Embodiment 18 The method of additional embodiment 17, wherein the MHC-I-dependent immunotherapeutic agent is selected from the group consisting of a checkpoint inhibitor, a cell-based therapy, and a cancer vaccine therapy.
Additional Embodiment 19 An affinity histochemical or affinity cytochemical method comprising: (a) contacting a cellular tumor sample with a biomarker-specific reagent under conditions that permit specific binding of the biomarker-specific reagent to the cellular tumor sample, wherein the biomarker-specific agent is one of a HLA-A biomarker specific reagent, a HLA-B biomarker specific reagent, or a HLA-C biomarker specific reagent; (b) removing unbound biomarker-specific reagent from the sample, thereby obtaining a labeled cellular tumor sample; and (c) contacting the labeled cellular tumor sample with a set of detection reagents which interact with the biomarker-specific reagent to facilitate deposition of a detectable moiety on the labeled cellular tumor sample.
Additional Embodiment 20 The affinity histochemical or affinity cytochemical method of additional embodiment 19, wherein the human HLA-A biomarker-specific reagent is a human HLA-A protein biomarker-specific reagent; wherein the human HLA-B biomarker-specific reagent is a human HLA-B protein biomarker-specific reagent; or wherein the human HLA-C biomarker-specific reagent is a human HLA-C protein biomarker-specific reagent.
Additional Embodiment 21 The affinity histochemical or affinity cytochemical method of additional embodiment 20, wherein the human HLA-A protein biomarker-specific reagent is an anti-human HLA-A antibody; wherein the human HLA-B biomarker-specific reagent is a human HLA-B protein biomarker-specific reagent; wherein the human HLA-C biomarker-specific reagent is a human HLA-C protein biomarker-specific reagent.
Additional Embodiment 22 The affinity histochemical or affinity cytochemical method of additional embodiment 19, wherein the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-CRNA biomarker-specific reagent.
Additional Embodiment 23 The affinity histochemical or affinity cytochemical method of any one of additional embodiments 19-22, wherein the cellular tumor sample comprises a tissue section.
Additional Embodiment 24 The affinity histochemical or affinity cytochemical method of any one of additional embodiments 19-22, wherein the cellular tumor sample comprises a cytology sample.
Additional Embodiment 25 The affinity histochemical or affinity cytochemical method of any one of additional embodiments 19-24, wherein the detectable moiety is selected from the group consisting of a chromogenic dye, a fluorophore, a mass spectrometer-detectable label, and a nucleic acid barcode.
Additional Embodiment 26 The affinity histochemical or affinity cytochemical method of any one of additional embodiments 19-25, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 27 The affinity histochemical or affinity cytochemical method of any one of additional embodiments 19-25, wherein the cellular tumor sample is derived from a tumor previously screened for TAP expression.
Additional Embodiment 28 An affinity histochemical or affinity cytochemical method comprising: (a) contacting a cellular tumor sample with a human pan-HLA biomarker-specific reagent under conditions that permit specific binding of the biomarker-specific reagent to the cellular tumor sample; (b) removing unbound biomarker-specific reagent from the sample, thereby obtaining a labeled cellular tumor sample; and (c) contacting the labeled cellular tumor sample with a set of detection reagents that interact with the biomarker-specific reagent to facilitate deposition of a detectable moiety on the labeled cellular tumor sample, wherein the human pan-HLA biomarker-specific reagent is a human pan-HLA protein biomarker-specific reagent.
Additional Embodiment 29 An affinity histochemical or affinity cytochemical method comprising: (a) contacting one or more cellular tumor samples with a human HLA-A biomarker-specific reagent, a human HLA-B biomarker-specific reagent, and a human HLA-C biomarker-specific reagent under conditions that permit specific binding of the HLA-A, HLA-B, and HLA-C biomarker-specific reagents to the one or more cellular tumor samples; removing unbound HLA-A, HLA-B, and HLA-C biomarker-specific reagent from the one or more cellular tumor samples, thereby obtaining one or more labeled cellular tumor samples; and (c) contacting the one or more labeled cellular tumor samples with a set of detection reagents that interact with the HLA-A, HLA-B, and HLA-C biomarker-specific reagents to facilitate deposition of a detectable moiety on the one or more labeled cellular tumor samples.
Additional Embodiment 30 The affinity histochemical or affinity cytochemical method of additional embodiment 29, wherein the same cellular tumor sample is contacted with the human HLA-A, HLA-B, and HLA-C biomarker-specific reagents.
Additional Embodiment 31 The affinity histochemical or affinity cytochemical method of additional embodiment 29, wherein different cellular tumor samples are contacted with the human HLA-A, HLA-B, and HLA-C biomarker-specific reagents.
Additional Embodiment 32 The affinity histochemical or affinity cytochemical method of additional embodiment 29, wherein the human HLA-A biomarker-specific reagent is an anti-human HLA-A antibody, the human HLA-B biomarker-specific reagent is an anti-human HLA-B antibody, and the human HLA-C biomarker-specific reagent is an anti-human HLA-C antibody.
Additional Embodiment 33 The affinity histochemical or affinity cytochemical method of additional embodiment 29, wherein the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-CRNA biomarker-specific reagent.
Additional Embodiment 34 An affinity histochemical or affinity cytochemical method comprising: (a) contacting a cellular tumor sample with a human HLA-A biomarker-specific reagent under conditions that permit specific binding of the biomarker-specific reagent to the cellular tumor sample; (b) contacting the cellular tumor sample with a human HLA-B biomarker-specific reagent under conditions that permit specific binding of the biomarker-specific reagent to the cellular tumor sample; and (c) contacting the cellular tumor sample with a human HLA-C biomarker-specific reagent under conditions that permit specific binding of the biomarker-specific reagent to the cellular tumor sample; wherein the human HLA-A biomarker-specific reagent is conjugated to a first detectable moiety; the human HLA-B biomarker-specific reagent is conjugated to a second detectable moiety; and the human HLA-C biomarker-specific reagent is conjugated to a third detectable moiety.
Additional Embodiment 35 The affinity histochemical or affinity cytochemical method of additional embodiment 34, further comprising: (d) contacting the cellular tumor sample with a set of detection reagents that interact with the human HLA-A biomarker-specific reagent to facilitate deposition of a first detectable moiety on the cellular tumor sample; (e) contacting the cellular tumor sample with a set of detection reagents that interact with the human HLA-B biomarker-specific reagent to facilitate deposition of a second detectable moiety on the cellular tumor sample; and (f) contacting the cellular tumor sample with a set of detection reagents that interact with the human HLA-C biomarker-specific reagent to facilitate deposition of a third detectable moiety on the cellular tumor sample.
Additional Embodiment 36 The affinity histochemical or affinity cytochemical method of any one of additional embodiments 34-35, wherein the human HLA-A biomarker-specific reagent is an anti-human HLA-A antibody, the human HLA-B biomarker-specific reagent is an anti-human HLA-B antibody, and the human HLA-C biomarker-specific reagent is an anti-human HLA-C antibody.
Additional Embodiment 37 The affinity histochemical or affinity cytochemical method of any one of additional embodiments 34-35, wherein the human HLA-A biomarker-specific reagent is a human HLA-A RNA biomarker-specific reagent; the HLA-B biomarker-specific reagent is a human HLA-B RNA biomarker-specific reagent; and the HLA-C biomarker-specific reagent is a human HLA-C RNA biomarker-specific reagent.
Additional Embodiment 38 The affinity histochemical or affinity cytochemical method of any one of additional embodiments 34-37, further comprising contacting the cellular tumor sample with one or more human tumor cell marker biomarker-specific reagents under conditions that permit specific binding of the one or more human tumor cell marker biomarker-specific reagents to the cellular tumor sample.
Additional Embodiment 39 The affinity histochemical or affinity cytochemical method of any one of additional embodiments 34-38, further comprising contacting the cellular tumor sample with a human B2M biomarker-specific reagent under conditions that permit specific binding of the human B2M biomarker-specific reagent to the cellular tumor sample.
Additional Embodiment 40 The affinity histochemical or affinity cytochemical method of additional embodiment 35, wherein the detectable moiety is selected from the group consisting of a chromogenic dye, a fluorophore, a mass spectrometer-detectable label, and a nucleic acid barcode.
Additional Embodiment 41 The affinity histochemical or affinity cytochemical method of any one of additional embodiments 34-40, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 42 The affinity histochemical or affinity cytochemical method of any one of additional embodiments 34-40, wherein the cellular tumor sample is derived from a tumor previously screened for TAP expression.
Additional Embodiment 43 An affinity histochemical or affinity cytochemical method comprising: (a) contacting a cellular tumor sample with a HLA biomarker-specific reagent under conditions that permit specific binding of the HLA biomarker-specific reagent to the cellular tumor sample; and (b) contacting the cellular tumor sample with a human tumor cell marker biomarker-specific reagent under conditions that permit specific binding of the human tumor cell marker biomarker-specific reagent to the cellular tumor sample; wherein the human HLA biomarker-specific reagent is conjugated to a first detectable moiety and the human tumor cell marker biomarker-specific reagent is conjugated to a second detectable moiety, wherein the first and second detectable moieties are different.
Additional Embodiment 44 The affinity histochemical or affinity cytochemical method of additional embodiment 43, further comprising (c) contacting the cellular tumor sample with a set of detection reagents that interact with the human HLA biomarker-specific reagent to facilitate deposition of a first detectable moiety on the cellular tumor sample; and (d) contacting the cellular tumor sample with a set of detection reagents that interact with the tumor cell marker biomarker-specific reagent to facilitate deposition of a second detectable moiety on the cellular tumor sample.
Additional Embodiment 45 The affinity histochemical or affinity cytochemical method of additional embodiment 43, wherein the HLA biomarker-specific reagent is selected from the group consisting of a human HLA-A protein biomarker-specific reagent, a human HLA-B protein biomarker-specific reagent, and a human HLA-C protein biomarker-specific reagent.
Additional Embodiment 46 The affinity histochemical or affinity cytochemical method of additional embodiment 43, wherein the HLA biomarker-specific reagent is a pan-HLA protein biomarker-specific reagent.
Additional Embodiment 47 The affinity histochemical or affinity cytochemical method of any one of additional embodiments 43-46, further comprising contacting the cellular tumor sample with a human B2M biomarker-specific reagent under conditions that permit specific binding of the human B2M biomarker-specific reagent to the cellular tumor sample.
Additional Embodiment 48 The affinity histochemical or affinity cytochemical method of any one of additional embodiments 43-47, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 49 An affinity histochemical or affinity cytochemical method comprising: (a) contacting a cellular tumor sample with one or more human biomarker-specific reagents under conditions that permit specific binding of the one or more human biomarker-specific reagents to the cellular tumor sample, wherein the one or more human biomarker-specific reagents are selected from the group consisting of a human TAP1 biomarker specific reagent and a human TAP2 biomarker specific reagent; (b) removing unbound one or more human biomarker-specific reagents from the cellular tumor sample, thereby obtaining a labeled cellular tumor sample; and (c) contacting the labeled cellular tumor sample with one or more sets of detection reagents that interact with bound one or more human biomarker-specific reagents to facilitate deposition of a detectable moiety on the labeled cellular tumor sample.
Additional Embodiment 50 The affinity histochemical or affinity cytochemical method of additional embodiment 49, wherein the human TAP1 biomarker-specific reagent is a human TAP1 protein biomarker-specific reagent; or wherein the human TAP2 biomarker-specific reagent is a human TAP2 protein biomarker-specific reagent.
Additional Embodiment 51 The affinity histochemical or affinity cytochemical method of additional embodiment 50, wherein the human TAP1 protein biomarker-specific reagent is an anti-human TAP1 antibody; or wherein the human TAP2 protein biomarker-specific reagent is an anti-human TAP2 antibody.
Additional Embodiment 52 The affinity histochemical or affinity cytochemical method of additional embodiment 49, wherein the cellular tumor sample is contacted with both the human TAP1 biomarker specific reagent and the human TAP21 biomarker specific reagent.
Additional Embodiment 53 The affinity histochemical or affinity cytochemical method of additional embodiment 49, wherein the human TAP1 biomarker-specific reagent and the human TAP2 biomarker-specific reagent are applied separately.
Additional Embodiment 54 The affinity histochemical or affinity cytochemical method of additional embodiment 49, wherein the human TAP1 biomarker-specific reagent and a human TAP2 biomarker-specific reagent are applied via a human pan-TAP biomarker-specific reagent cocktail.
Additional Embodiment 55 The affinity histochemical or affinity cytochemical method of any one of additional embodiments 49-54, wherein the cellular tumor sample is a tissue section.
Additional Embodiment 56 The affinity histochemical or affinity cytochemical method of any one of additional embodiments 49-54, wherein the cellular tumor sample is a cytology sample.
Additional Embodiment 57 The affinity histochemical or affinity cytochemical method of any one of additional embodiments 49-56, wherein the detectable moiety is selected from the group consisting of a chromogenic dye, a fluorophore, a mass spectrometer-detectable label, and a nucleic acid barcode.
Additional Embodiment 58 The affinity histochemical or affinity cytochemical method of any one of additional embodiments 49-57, wherein the cellular tumor sample is derived from a tumor previously determined to express one or more of HLA-A, HLA-B, or HLA-C.
Additional Embodiment 59 The affinity histochemical or affinity cytochemical method of any one of additional embodiments 49-58, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 60 An affinity histochemical or affinity cytochemical method comprising: (a) contacting a first cellular tumor sample with a human TAP1 biomarker-specific reagent under conditions that permit specific binding of the biomarker-specific reagent to the first cellular tumor sample; (b) removing unbound biomarker-specific reagent from the first cellular sample, thereby obtaining a first labeled cellular tumor sample; (c) contacting a second cellular tumor sample with a human TAP2 biomarker-specific reagent under conditions that permit specific binding of the biomarker-specific reagent to the second cellular tumor sample; and (d) removing unbound biomarker-specific reagent from the second cellular tumor sample, thereby obtaining a second labeled cellular tumor sample.
Additional Embodiment 61 The affinity histochemical or affinity cytochemical method of additional embodiment 60, further comprising: (e) contacting the first labeled cellular tumor sample with a set of detection reagents that interact with the biomarker-specific reagent to facilitate deposition of a detectable moiety on the first labeled cellular tumor sample; and (f) contacting the second labeled cellular tumor sample with a set of detection reagents that interact with the biomarker-specific reagent to facilitate deposition of a detectable moiety on the second labeled cellular tumor sample.
Additional Embodiment 62 The affinity histochemical or affinity cytochemical method of any one of additional embodiments 60-61, wherein the human TAP1 biomarker-specific reagent is a human TAP1 protein biomarker-specific reagent; or wherein the human TAP2 biomarker-specific reagent is a human TAP2 protein biomarker-specific reagent.
Additional Embodiment 63 The affinity histochemical or affinity cytochemical method of additional embodiment 62, wherein the human TAP1 protein biomarker-specific reagent is an anti-human TAP1 antibody; or wherein the human TAP2 protein biomarker-specific reagent is an anti-human TAP2 antibody.
Additional Embodiment 64 The affinity histochemical or affinity cytochemical method of any one of additional embodiments 60-61, wherein the human TAP1 biomarker-specific reagent is a human TAP1 RNA biomarker-specific reagent and wherein the human TAP2 biomarker-specific reagent is a human TAP2 RNA biomarker-specific reagent.
Additional Embodiment 65 The affinity histochemical or affinity cytochemical method of any one of additional embodiments 60-64, wherein the first and second cellular tumor samples are tissue sections.
Additional Embodiment 66 The affinity histochemical or affinity cytochemical method of any one of additional embodiments 60-64, wherein the first and second cellular tumor samples are cytology samples.
Additional Embodiment 67 The affinity histochemical or affinity cytochemical method of any one of additional embodiments 60-66, wherein the first and second cellular tumor samples are derived from a tumor previously determined to express one or more of HLA-A, HLA-B, or HLA-C.
Additional Embodiment 68 The affinity histochemical or affinity cytochemical method of any one of additional embodiments 61-67, wherein the first and second cellular tumor samples are derived from a tumor previously screened by a tumor mutational screen selected from the group consisting of a tumor mutational burden screen, a microsatellite stability screen, and a tumor previously screened by a mismatch repair screen.
Additional Embodiment 69 An affinity histochemical or affinity cytochemical method comprising: (a) contacting a cellular tumor sample with a human TAP1 biomarker-specific reagent under conditions that permit specific binding of the biomarker-specific reagent to the cellular tumor sample; and (b) contacting the cellular tumor sample with a human TAP2 biomarker-specific reagent under conditions that permit specific binding of the biomarker-specific reagent to the cellular tumor sample; wherein the human TAPI biomarker-specific reagent is conjugated to a first detectable moiety; and the human TAP2 biomarker-specific reagent is conjugated to a second detectable moiety.
Additional Embodiment 70 The affinity histochemical or affinity cytochemical method of additional embodiment 69, further comprising: (c) contacting the cellular tumor sample with a set of detection reagents that interact with the human TAP1 biomarker-specific reagent to facilitate deposition of a first detectable moiety on the cellular tumor sample; and (d) contacting the cellular tumor sample with a set of detection reagents that interact with the human TAP2 biomarker-specific reagent to facilitate deposition of a second detectable moiety on the cellular tumor sample.
Additional Embodiment 71 The affinity histochemical or affinity cytochemical method of any one of additional embodiments 69-70, wherein the human TAP1 biomarker-specific reagent is a human TAP1 protein biomarker-specific reagent; or wherein the human TAP2 biomarker-specific reagent is a human TAP2 protein biomarker-specific reagent.
Additional Embodiment 72 The affinity histochemical or affinity cytochemical method of additional embodiment 71, wherein the human TAP1 protein biomarker-specific reagent is an anti-human TAP1 antibody; or wherein the human TAP2 protein biomarker-specific reagent is an anti-human TAP2 antibody.
Additional Embodiment 73 The affinity histochemical or affinity cytochemical method of additional embodiment 69, wherein the detectable moiety is selected from the group consisting of a chromogenic dye, a fluorophore, a mass spectrometer-detectable label, and a nucleic acid barcode.
Additional Embodiment 74 The affinity histochemical or affinity cytochemical method of any one of additional embodiments 69-73, wherein the cellular tumor sample is derived from a tumor previously determined to express one or more of HLA-A, HLA-B, or HLA-C.
Additional Embodiment 75 The affinity histochemical or affinity cytochemical method of any one of additional embodiments 69-73, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 76 An affinity histochemical or affinity cytochemical method comprising: (a) contacting a cellular tumor sample with a human TAP biomarker-specific reagent under conditions that permit specific binding of the human TAP biomarker-specific reagent to the cellular tumor sample; and (b) contacting the cellular tumor sample with a tumor cell marker biomarker-specific reagent under conditions that permit specific binding of the human tumor cell marker biomarker-specific reagent to the cellular tumor sample; wherein the human TAP biomarker-specific reagent is conjugated to a first detectable moiety and the human tumor cell marker biomarker-specific reagent is conjugated to a second detectable moiety, wherein the first and second detectable moieties are different.
Additional Embodiment 77 The affinity histochemical or affinity cytochemical method of additional embodiment 76, further comprising: (c) contacting the cellular tumor sample with a set of detection reagents that interact with the human TAP biomarker-specific reagent to facilitate deposition of a first detectable moiety on the cellular tumor sample; and (d) contacting the cellular tumor sample with a set of detection reagents that interact with the tumor cell marker biomarker-specific reagent to facilitate deposition of a second detectable moiety on the cellular tumor sample.
Additional Embodiment 78 The affinity histochemical or affinity cytochemical method of any one of additional embodiments 76-77, wherein the human TAP biomarker-specific reagent is an anti-human TAP1 antibody or an anti-human TAP2 antibody.
Additional Embodiment 79 The affinity histochemical or affinity cytochemical method of any one of additional embodiments 76-77, wherein the human TAP biomarker-specific reagent is a human pan-TAP protein biomarker-specific reagent.
Additional Embodiment 80 The affinity histochemical or affinity cytochemical method of any one of additional embodiments 76-79, wherein the cellular tumor sample is derived from an epithelial tumor and the human tumor cell biomarker-specific reagent is a human cytokeratin biomarker-specific reagent.
Additional Embodiment 81 The affinity histochemical or affinity cytochemical method of additional embodiment 80, wherein the human cytokeratin biomarker-specific reagent is a pan-cytokeratin antibody cocktail.
Additional Embodiment 82 The affinity histochemical or affinity cytochemical method of any one of additional embodiments 76-79, wherein the cellular tumor sample is derived from a mesenchymal tumor and the human tumor cell marker biomarker-specific reagent is a vimentin biomarker-specific reagent.
Additional Embodiment 83 The affinity histochemical or affinity cytochemical method of any one of additional embodiments 76-79, wherein the cellular tumor sample is derived from a tumor previously determined to express one or more of HLA-A, HLA-B, or HLA-C.
Additional Embodiment 84 The affinity histochemical or affinity cytochemical method of any one of additional embodiments 76-79, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 85 The affinity histochemical or affinity cytochemical method of any one of additional embodiments 76-79, wherein the cellular tumor sample is derived from a tumor of lymphoid origin and the human tumor cell marker biomarker-specific reagent is a CD45 biomarker-specific reagent.
Additional Embodiment 86 An affinity histochemical or affinity cytochemical method comprising: (a) contacting a cellular tumor sample with a human TAP biomarker-specific reagent under conditions that permit specific binding of the human TAP biomarker-specific reagent to the cellular tumor sample; and (b) contacting the cellular tumor sample with either or both of a human HLA biomarker-specific reagent and/or a human B2M biomarker-specific reagent under conditions that permit specific binding of the human HLA and/or human B2M biomarker-specific reagents to the cellular tumor sample.
Additional Embodiment 87 The affinity histochemical or affinity cytochemical method of additional embodiment 86, wherein the human TAP biomarker-specific reagent is conjugated to a first detectable moiety; and the human HLA biomarker-specific reagent is conjugated to a second detectable moiety, wherein the first and second detectable moieties are different.
Additional Embodiment 88 The affinity histochemical or affinity cytochemical method of additional embodiment 86, further comprising: (c) contacting the cellular tumor sample with a set of detection reagents that interact with the human TAP biomarker-specific reagent to facilitate deposition of a first detectable moiety on the cellular tumor sample; and (d) contacting the cellular tumor sample with a set of detection reagents that interact with the HLA biomarker-specific reagent and/or the human B2M biomarker-specific reagent to facilitate deposition of a second detectable moiety on the cellular tumor sample.
Additional Embodiment 89 The affinity histochemical or affinity cytochemical method of any one of additional embodiments 86-88, wherein the human TAP biomarker-specific reagent is an anti-human TAP1 antibody or an anti-human TAP2 antibody.
Additional Embodiment 90 The affinity histochemical or affinity cytochemical method of any one of additional embodiments 86-88, wherein the human TAP biomarker-specific reagent is a human pan-TAP protein biomarker-specific reagent.
Additional Embodiment 91 The affinity histochemical or affinity cytochemical method of any one of additional embodiments 86-88, wherein the human HLA biomarker-specific reagent is selected from the group consisting of a human HLA-A biomarker-specific reagent, a human HLA-B biomarker-specific reagent, and a human HLA-C biomarker-specific reagent.
Additional Embodiment 92 The affinity histochemical or affinity cytochemical method of any one of additional embodiments 86-88, wherein the human HLA biomarker-specific reagent is a human pan-HLA protein biomarker-specific reagent.
Additional Embodiment 93 The affinity histochemical or affinity cytochemical method of additional embodiment 86, wherein the cellular tumor sample is contacted with both a human TAP1 biomarker-specific reagent and a human TAP2 biomarker-specific reagent, wherein the human TAP1 and TAP2 biomarker-specific reagents are each conjugated to different detectable moieties.
Additional Embodiment 94 The affinity histochemical or affinity cytochemical method of any one of additional embodiments 86-93, wherein the cellular tumor sample is derived from a tumor previously screened by a tumor mutational screen selected from the group consisting of a tumor mutational burden screen, a microsatellite stability screen, and a tumor previously screened by a mismatch repair screen.
Additional Embodiment 95 A 2-plex chromogenic immunohistochemical assay comprising: (a) contacting a first tissue section of a tumor with an anti-human TAP monoclonal antibody under conditions that permit specific binding of the anti-human TAP monoclonal antibody to the first tissue section; (b) contacting the first tissue section with a set of detection reagents that interact with the anti-human TAP monoclonal antibody bound to the tissue section to chromogenically deposit a first brightfield dye on the tissue section; (c) contacting a second tissue section of the tumor with either an anti-human HLA monoclonal antibody or an anti-human B2M monoclonal antibody under conditions that permit specific binding of the anti-human HLA monoclonal antibody or the anti-human B2M monoclonal antibody to the second tissue section; and (d) contacting the second tissue section with a set of detection reagents that interact with the anti-human HLA monoclonal antibody or the anti-human B2M monoclonal antibody bound to the second tissue section to chromogenically deposit a second brightfield dye on the second tissue section, wherein the first and second brightfield dyes are separately detectable on the tissue section.
Additional Embodiment 96 The affinity histochemical or affinity cytochemical method of additional embodiment 95, wherein the human TAP biomarker-specific reagent is an anti-human TAP1 antibody or an anti-human TAP2 antibody.
Additional Embodiment 97 The affinity histochemical or affinity cytochemical method of additional embodiment 95, wherein the human TAP biomarker-specific reagent is a human pan-TAP protein biomarker-specific reagent.
Additional Embodiment 98 The affinity histochemical or affinity cytochemical method of additional embodiment 95, wherein the human HLA biomarker-specific reagent is selected from the group consisting of a human HLA-A biomarker-specific reagent, a human HLA-B biomarker-specific reagent, and a human HLA-C biomarker-specific reagent.
Additional Embodiment 99 The affinity histochemical or affinity cytochemical method of additional embodiment 95, wherein the human HLA biomarker-specific reagent is a human pan-HLA protein biomarker-specific reagent.
Additional Embodiment 100 A method of quantifying a percentage of TAP biomarker positive tumors cells and a percentage of TAP biomarker positive immune cells in a cellular tumor sample comprising:
   staining dissociated cells within a first aliquot of the cellular tumor sample for the presence of the TAP biomarker and a tumor cell biomarker;
   staining dissociated cells within a second aliquot of the cellular tumor sample for the presence of the TAP biomarker and an immune biomarker;
   obtaining fluorescence data for the stained dissociated cells within each of the first and second aliquots;
   based on the obtained fluorescence data, identifying a TAP biomarker positive tumor cell population within the first aliquot and a TAP biomarker positive immune cell population in the second aliquot; and
   quantifying the percentage of TAP biomarker positive tumor cells and the percentage of TAP biomarker positive immune cells within the cellular tumor sample.
Additional Embodiment 101 The method of additional embodiment 100, wherein the tumor cell biomarker is an epithelial marker.
Additional Embodiment 102 The method of additional embodiment 101, wherein the epithelial marker is a cytokeratin.
Additional Embodiment 103 The method of additional embodiment 102, wherein the cytokeratin is one of a specific cytokeratin marker or pan-cytokeratin.
Additional Embodiment 104 The method of additional embodiment 100, wherein the immune cell biomarker is selected from the group consisting of CD45, CD3, CD4, CD8, CD20, CD 25, CD19, CD163, CD68, CD69 and CD103.
Additional Embodiment 105 The method of additional embodiment 100, wherein the obtained fluorescence data comprises scatter plots of fluorescence intensity versus side scatter content.
Additional Embodiment 106 The method of additional embodiment 100, wherein the identifying of the TAP biomarker positive tumor cell population comprises performing a first sequential gating operation on the obtained fluorescence data for the stained dissociated cells within the first aliquot; and wherein the identifying of the TAP biomarker positive immune cell population comprises performing a second sequential gating operation on the obtained fluorescence data for the stained dissociated cells within the second aliquot.
Additional Embodiment 107 All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.
Additional Embodiment 108 Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

### REFERENCES

Au et al., “Determinants of Anti-PD1 Response and Resistance in Clear Cell Renal Cell Carcinoma,” Cancer Cell. 2021 Nov. 8; 39 (11): 1497-1518.e11. doi: 10.1016/j.ccell.2021.10.001. Epub 2021 Oct. 28. PMID: 34715028; PMCID: PMC8599450.
Baharlou et al., Mass Cytometry Imaging for the Study of Human Diseases—Applications and Data Analysis Strategies, Frontiers in Immunology, 2019, Vol. 10, Art. 2657.
Bodenmiller, Multiplexed Epitope-Based Tissue Imaging for Discovery and Healthcare Applications, Cell Systems, 2016, Vol. 2, Issue 4, pp. 225-38.
Gigoux & Wolchok, Refusing to TAP out: 16 new human TEIPPs identified, Journal of Experimental Medicine, 2018, Vol. 215, Issue 9, pp. 2233-34).
Goodman et al., Tumor Mutational Burden as an Independent Predictor of Response to Immunotherapy in Diverse Cancer, Molecular Cancer Therapies, 2017, Vol. 16, Issue 11, pp. 2598-2608.
Gorris et al., Eight-Color Multiplex Immunohistochemistry for Simultaneous Detection of Multiple Immune Checkpoint Molecules within the Tumor Microenvironment, 2018, Journal of Immunology, Vol. 200, Issue 1, pp. 347-54.
Green et al., A review of immune checkpoint blockade therapy in endometrial cancer, American Society of Clinical Oncology Educational Book 40 (Mar. 26, 2020), pp. 238-244).
Hofman et al., Multiplexed Immunohistochemistry for Molecular and Immune Profiling in Lung Cancer—Just About Ready for Prime-Time?, Cancers, 2019, Vol. 11, No. 283.
Hong et al., Biomarkers for Chimeric Antigen Receptor T Cell Therapy in Acute Lymphoblastic Leukemia: Prospects for Personalized Management and Prognostic Prediction, Frontiers in Immunology, 2021, 12:627764. doi: 10.3389/fimmu.2021.627764.
Ide et al., Chromogenic Multiplex Immunohistochemistry Reveals Modulation of the Immune Microenvironment Associated with Survival in Elderly Patients with Lung Adenocarcinoma, Cancers (Basel), 2018, Vol. 10, Issue 9, No. 326.
Jamalzadeh et al., QuantISH: RNA in situ hybridization image analysis framework for quantifying cell type-specific target RNA expression and variability, Laboratory Investigation, 2022, https://doi.org/10.1038/s41374-022-00743-5.
Jenson et al., A novel quantitative immunohistochemistry method for precise protein measurements directly in formalin-fixed, paraffin-embedded specimens: analytical performance measuring HER2, Modern Pathology, 2017, Vol. 30, pp. 180-93 (describing an exemplary quantitative IHC method).
Klempner et al., Tumor Mutational Burden as a Predictive Biomarker for Response to Immune Checkpoint Inhibitors: A Review of Current Evidence, Oncologist, 2020, Vol. 25, Issue 1, e147-e159.
Le et al., PD-1 Blockade in Tumors with Mismatch-Repair Deficiency, New England Journal of Medicine, 2015, Vol. 372, Issue 26, pp. 2509-20.
Levenson, Immunohistochemistry and mass spectrometry for highly multiplexed cellular molecular imaging, Laboratory Investigation, 2015, Vol. 95, pp. 397-405.
Marin-Acevedo et al., Next generation of immune checkpoint inhibitors and beyond, Journal of Hematology and Oncology, 2021, Vol. 14, Art. No. 45.
Marjit et al., Identification of non-mutated neoantigens presented by TAP-deficient tumors, Journal of Experimental Medicine, 2018, Vol. 215, Issue 9, pp. 2325-37.
Morrison et al., Brightfield multiplex immunohistochemistry with multispectral imaging, Laboratory Investigation, 2020, Vol. 100, pp. 1124-36.
Parra et al., State-of-the-Art of Profiling Immune Contexture in the Era of Multiplexed Staining and Digital Analysis to Study Paraffin Tumor Tissues, Cancers, 2019, Vol. 11, Issue 2, No. 247.
Ptacek et al., Multiplexed ion beam imaging (MIBI) for characterization of the tumor microenvironment across tumor types, Laboratory Investigation, 2020, Vol. 100, pp. 1111-1123.
Sahin et al., Immune checkpoint inhibitors for the treatment of MSI-H/MMR-D colorectal cancer and a perspective on resistance mechanisms, British Journal of Cancer, 2019, Vol. 121, pp 809-818.
Seliger et al., Characterization of human lymphocyte antigen class I antigen-processing machinery defects in renal cell carcinoma lesions with special emphasis on transporter-associated with antigen-processing down-regulation, Clinical Cancer Research, 2003, Vol. 9, Issue 5, pp. 1721-27).
Shindo et al., Novel Biomarkers for Personalized Cancer Immunotherapy, Cancers (Basel), Vol. 11, Issue 9, Art. 1223.
Stack et al., Multiplexed immunohistochemistry, imaging, and quantitation: A review, with an assessment of Tyramide signal amplification, multispectral imaging, and multiplex analysis, 2014, Vol. 70, Issue 1, pp. 46-58.
Suekane et al., Identification of biomarkers for personalized peptide vaccination in 2,588 cancer patients, International Journal of Oncology, 2020, Vol. 56, Issue 6, pp. 1479-89.
Sun et al., Resistance to PD-1/PD-L1 blockade cancer immunotherapy: mechanisms, predictive factors, and future perspectives, Biomarker Research, 2020, Vol. 8, Art. No. 35.
Tan et al., Overview of multiplex immunohistochemistry/immunofluorescence techniques in the era of cancer immunotherapy, Cancer Communications (London), vol. 40, issue 4, pp. 135-53.
van Belzen & Kesmir, Immune biomarkers for predicting response to adoptive cell transfer as cancer treatment, Immunogenetics, 2019, Vol. 71, Issue 2, pp. 71-86
Yarchoan et al., PD-L1 expression and tumor mutational burden are independent biomarkers in most cancers, JCI Insight, 2019, Vol. 4, Issue 6, e126908 (“Yarchoan I”).
Yarchoan et al., Tumor Mutational Burden and Response Rate to PD-1 Inhibition. N Engl J Med. 2017 Dec. 21; 377 (25): 2500-2501 (“Yarchoan II”).
Zhu & Liu, The Role of Neoantigens in Cancer Immunotherapy, Frontiers in Oncology, 2021, Vol. 11, DOI=10.3389/fonc.2021.682325.

1. An affinity histochemical or affinity cytochemical method comprising: (a) contacting a first cellular tumor sample with one or more human biomarker-specific reagents under conditions that permit specific binding of the one or more human biomarker-specific reagents to the first cellular tumor sample, wherein the one or more human biomarker-specific reagents are selected from the group consisting of a human TAP1 biomarker specific reagent and a human TAP2 biomarker specific reagent; (b) removing unbound one or more human biomarker-specific reagents from the first cellular tumor sample, thereby obtaining a labeled first cellular tumor sample; and (c) contacting the labeled first cellular tumor sample with one or more sets of detection reagents that interact with bound one or more human biomarker-specific reagents to facilitate deposition of a first detectable moiety on the labeled first cellular tumor sample, wherein the human TAP1 biomarker-specific reagent is a human TAP1 protein biomarker-specific reagent; or wherein the human TAP2 biomarker-specific reagent is a human TAP2 protein biomarker-specific reagent.

2. The affinity histochemical or affinity cytochemical method of claim 1, wherein the first cellular tumor sample is contacted with both the human TAP1 biomarker specific reagent and the human TAP2 biomarker specific reagent.

3. The affinity histochemical or affinity cytochemical method of claim 2, wherein the human TAPI biomarker-specific reagent and the human TAP2 biomarker-specific reagent are applied separately.

4. The affinity histochemical or affinity cytochemical method of claim 1, wherein the human TAP 1 biomarker-specific reagent and a human TAP2 biomarker-specific reagent are applied via a human pan-TAP biomarker-specific reagent cocktail.

5. The affinity histochemical or affinity cytochemical method of claim 1, wherein the first cellular tumor sample is a tissue section.

6. The affinity histochemical or affinity cytochemical method of claim 1, wherein the first cellular tumor sample is a cytology sample.

7. The affinity histochemical or affinity cytochemical method of claim 1, wherein the first cellular tumor sample is derived from a tumor previously determined to express one or more of HLA-A, HLA-B, or HLA-C.

8. The affinity histochemical or affinity cytochemical method of claim 1, wherein the first cellular tumor sample is derived from a tumor previously screened by a tumor mutational screen selected from the group consisting of a tumor mutational burden screen, a microsatellite stability screen, and a tumor previously screened by a mismatch repair screen.

9. The affinity histochemical or affinity cytochemical method of claim 1, wherein the first cellular tumor sample is derived from a tumor previously determined to be a deficient MMR (“dMMR”) tumor, a microsatellite instability-high (“MSI-H”) tumor, and/or a tumor mutational burden-high (“TMB-H”) tumor.

10. The affinity histochemical or affinity cytochemical method of claim 1, wherein the first cellular tumor sample is derived from a tumor previously determined to be a proficient MMR (“pMMR”) tumor, a microsatellite instability-low (“MSI-L”) tumor, and/or a tumor mutational burden-low (“TMB-L”) tumor.

11. The affinity histochemical or affinity cytochemical method of claim 1, wherein the human TAP1 protein biomarker-specific reagent comprises a heavy chain variable domain (V_{H}) sequence having at least 90% sequence identity to SEQ ID NO: 8; and a light chain variable domain (V_{L}) having at least 90% sequence identity to SEQ ID NO: 9.

12. The affinity histochemical or affinity cytochemical method of claim 11, wherein the human TAP1 protein biomarker-specific reagent comprises a heavy chain CDR1 having SEQ ID NO: 11; a heavy chain CDR2 having SEQ ID NO: 13; a heavy chain CDR3 having SEQ ID NO: 14; a light chain CDR1 having SEQ ID NO: 18; a light chain CDR2 having SEQ ID NO: 20; and a light chain CDR3 having SEQ ID NO: 22.

13. The affinity histochemical or affinity cytochemical method of claim 1, further comprising contacting the first cellular tumor sample with either an anti-human HLA monoclonal antibody or an anti-human B2M monoclonal antibody under conditions that permit specific binding of the anti-human HLA monoclonal antibody or the anti-human B2M monoclonal antibody to the first cellular tumor sample; and contacting the first cellular tumor sample with a set of detection reagents that interact with the anti-human HLA monoclonal antibody or the anti-human B2M monoclonal antibody bound to the first cellular tumor sample to deposit a second detectable moiety of the labeled first cellular tumor sample, wherein the first and second detectable moieties are different, and wherein the human HLA biomarker-specific reagent is selected from the group consisting of a human HLA-A biomarker-specific reagent, a human HLA-B biomarker-specific reagent, and a human HLA-C biomarker-specific reagent.

14. The affinity histochemical or affinity cytochemical method of claim 13, wherein the human HLA biomarker-specific reagent is a human pan-HLA protein biomarker-specific reagent.

15. An anti-TAP1 antibody or antigen-binding portion thereof comprising a heavy chain variable domain (V_{H}) sequence having at least 95% sequence identity to SEQ ID NO: 8; and a light chain variable domain (V_{L}) having at least 95% sequence identity to SEQ ID NO: 9; wherein the anti-TAP1 antibody or the antigen-binding portion thereof comprises a heavy chain CDR1 having SEQ ID NO: 11; a heavy chain CDR2 having SEQ ID NO: 13; a heavy chain CDR3 having SEQ ID NO: 14; a light chain CDR1 having SEQ ID NO: 18; a light chain CDR2 having SEQ ID NO: 20; and a light chain CDR3 having SEQ ID NO: 22.

16. A method of determining whether a tumor sample derived from a subject should be referred for a tumor mutational screen, the method comprising evaluating, in cells of the tumor sample, (i) an expression of the constituent elements of a transporter associated with antigen processing (TAP) complex, and (ii) an expression of the constituent elements of the major histocompatibility complex class I (MHC); wherein the tumor sample is referred for the tumor mutational screen when the tumor sample is evaluated to be MHC-I(+) and TAP(+).

17. The method of claim 16, wherein the tumor mutational screen is selected from the group consisting of mismatch repair (MMR) screening, microsatellite instability (MSI) screening, and tumor mutational burden screening (TMB).

18. The method of claim 17, wherein if the tumor sample is evaluated to be MMR deficient (dMMR), MSI high, and/or TMB high, the tumor sample is likely to respond to an MCH-I dependent immunotherapeutic agent.

19. The method of claim 17, wherein if the tumor sample is evaluated to be MMR proficient (pMMR), MSI low, and/or TMB low, the tumor sample is unlikely to respond to an MCH-I dependent immunotherapeutic agent.

20. The method of claim 18, wherein the MCH-I dependent immunotherapeutic agent is selected from the group consisting of a checkpoint inhibitor, a cell-based therapy, and a cancer vaccine therapy.


SEQUENCE LISTING (23 sequences)

SEQ ID NO: 1 - protein, 748 aa, Homo sapiens
MASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 2 - protein, 686 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

SEQ ID NO: 3 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 4 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 5 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 6 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 7 - protein, 15 aa, Homo sapiens
DGKPLPQYEHRYLHR

SEQ ID NO: 8 - protein, 135 aa, Oryctolagus sp.
METGLRWLLLVAVLKGVQCQSVKESEGGLFKPTDTLTLTCTVSGFSLSSYEVIWVRQAPGKGLEWIGNIGWRDTTFYANWAKSRSTFTRDTNLNTVTLKMTSLTAADTATYFCARDNGYFGIDYWGPGTLVTVSS

SEQ ID NO: 9 - protein, 74 aa, Oryctolagus sp.
MDTRAPTQLLGLLLLWLPGARCADIVMTQTPASVEAAVGGTVTIKCQASQSISNYLAWYQQKPGQPPKLLIYKA

SEQ ID NO: 10 - protein, 24 aa, Oryctolagus sp.
QSVKESEGGLFKPTDTLTLTCTVS

SEQ ID NO: 11 - protein, 8 aa, Oryctolagus sp.
GFSLSSYE

SEQ ID NO: 12 - protein, 17 aa, Oryctolagus sp.
VIWVRQAPGKGLEWIGN

SEQ ID NO: 13 - protein, 7 aa, Oryctolagus sp.
IGWRDTT

SEQ ID NO: 14 - protein, 38 aa, Oryctolagus sp.
FYANWAKSRSTFTRDTNLNTVTLKMTSLTAADTATYFC

SEQ ID NO: 15 - protein, 11 aa, Oryctolagus sp.
ARDNGYFGIDY

SEQ ID NO: 16 - protein, 11 aa, Oryctolagus sp.
WGPGTLVTVSS

SEQ ID NO: 17 - protein, 26 aa, Oryctolagus sp.
DIVMTQTPASVEAAVGGTVTIKCQAS

SEQ ID NO: 18 - protein, 6 aa, Oryctolagus sp.
QSISNY

SEQ ID NO: 19 - protein, 17 aa, Oryctolagus sp.
LAWYQQKPGQPPKLLIY

SEQ ID NO: 20 - skipped

SEQ ID NO: 21 - protein, 36 aa, Oryctolagus sp.
TLASGVPSRFSGSGSGTQFTLTISGVQCDDAATYYC

SEQ ID NO: 22 - protein, 12 aa, Oryctolagus sp.
QQDFNYRNIENV

SEQ ID NO: 23 - protein, 10 aa, Oryctolagus sp.
FGGGTGVVVK